(19) United States
(12) Patent Application Publication
(10) Pub. No.: US 2005/0130203 A1
(43) Pub. Date: Jun. 16, 2005

(54) Novel GABAB receptor DNA sequences

(75) Inventors: Qingyun Liu, North Wales, PA (US); Terrence McDonald, Langhorne, PA (US); Timothy P. Bonnert, Hartfordshire (GB); Gordon Ng, Point-Claire (CA); Lee F. Kolakowski JR., Timonium, MD (US); Janet Clark, Bethesda, MD (US); Tom I. Bonner, Bethesda, MD (US)

Correspondence Address: Shelley P.M. Fussey, Ph.D.;WILLIAMS, MORGAN & AMERSON, P.C., Suite 1100, 10333 Richmond, Houston, TX, 77042, US

(73) Assignees: Board of Regents, The University of Texas System; Merck & Co., Inc.; Merck Frosst Canada & Co.; The Government of the USA as represented by the Secretary, Department of Health and Services

(21) Appl. No.: 10/983,198

(22) Filed: Nov. 4, 2004

Related U.S. Application Data
(63) Continuation of application No. 09/601,582, filed on Dec. 4, 2000.
(60) Provisional application No. 60/073,767, filed on Feb. 5, 1998.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

DNA encoding a novel human GABA_{B} receptor subunit, HG20, as well as the protein encoded by the DNA, is provided. Also provided is DNA encoding a novel murine GABA_{B} receptor subunit, GABA_{B}R1a, as well as the protein encoded by the DNA. Heterodimers of HG20 protein and GABA_{B}R1a protein that form a functional GABA_{B} receptor are disclosed. Methods of identifying agonists and antagonists of the GABA_{B} receptor are also provided.

### CROSS-REFERENCE TO RELATED APPLICATIONS

This application claims the benefit of U.S. Provisional Application No. 60/073,767, filed Feb. 5, 1998, the contents of which-are incorporated herein by reference in their entirety.

### STATEMENT REGARDING FEDERALLY-SPONSORED R&D

Not applicable.

### REFERENCE TO MICROFICHE APPENDIX

Not applicable.

### FIELD OF THE INVENTION

The present invention is directed to a novel human DNA sequence encoding HG20, a subunit of the GABA_{B} receptor, the protein encoded by the DNA, and uses thereof. The present invention also is directed to the murine GABA_{B}R1a subunit of the GABA_{B} receptor as well as to methods of combining an HG20 subunit with a GABA_{B}R1a subunit to form a GABA_{B} receptor having functional activity.

### BACKGROUND OF THE INVENTION

Amino acids such as glutamic acid, γ-amino-butyric acid (GABA), and glycine are neurotransmitters that bind to specific receptors in the vertebrate nervous system and mediate synaptic transmission. Of these amino acids, GABA is the most widely distributed amino acid inhibitory neurotransmitter in the vertebrate central nervous system. The biological activities of GABA are mediated by three types of GABA receptors: ionotropic GABA_{A} receptors, metabotropic GABA_{B} receptors, and ionotropic GABA_{C} receptors. Each type of receptor has its own characteristic molecular structure, pattern of gene expression, agonist and antagonist mediated pharmacological effects, and spectrum of physiological activities.

GABA_{A} receptors mediate fast synaptic inhibition. They are heterooligomeric proteins (most likely pentamers) containing α, β, γ, and perhaps δ, subunits that function as ligand-gated Cl⁻ channels and have binding sites for benzodiazepines, barbiturates, and neuroactive steroids. Bicuculline is a widely used antagonist of GABA_{A} receptors. Bicuculline is selective for GABA_{A} receptors in that it has no effect on GABA_{B} or GABA_{C} receptors. The expression of GABA_{A} receptors has been observed in a variety of brain structures (see. e.g., McKernan & Whiting, 1996, Trends Neurosci. 16:139-143; Sequier et al., 1988, Proc. Natl. Acad. Sci. USA 85:7815-7819).

GABA_{C} receptors are ligand-gated Cl⁻ channels found in the vertebrate retina. They can be distinguished from GABA_{A} and GABA_{B} receptors in that they are insensitive to the GABA_{A} receptor antagonist bicuculline and the GABA_{B} receptor agonist (−)baclofen but are selectively activated by cis-4-aminocrotonic acid. GABA_{C} receptors are composed of homooligomers of a category of GABA receptor subunits known as “ρ” subunits, the best-studied of which are ρ1 and ρ2. ρ1 and ρ2 share 74% amino acid sequence identity but are only about 30-38% identical in amino acid sequence when compared to GABA_{A} receptor subunits. For a review of GABA_{C} receptors, see Bormann & Feigenspan, 1995, Trends Neurosci. 18:515-518.

GABA_{B} receptors play a role in the mediation of late inhibitory postsynaptic potentials (IPSPs). GABA_{B} receptors belong to the superfamily of seven transmembrane-spanning G-protein coupled receptors that are coupled through G-proteins to neuronal K⁺ or Ca⁺⁺ channels. GABA_{B} receptors are coupled through G-proteins to neuronal K⁺ or Ca⁺⁺ channels, and receptor activation increases K⁺ or decreases Ca⁺⁺ conductance and also inhibits or potentiates stimulated adenylyl cyclase activity. The expression of GABA_{B} receptors is widely distributed in the mammalian brain (e.g., frontal cortex, cerebellar molecular layer, interpeduncular nucleus) and has been observed in many peripheral organs as well.

A large number of phammacological activities have been attributed to GABA_{B} receptor activation, e.g., analgesia; hypothermia; catatonia; hypotension; reduction of memory consolidation and retention; and stimulation of insulin, growth hormone, and glucagon release (see Bowery, 1989, Trends Pharmacol. Sci. 10:401-407, for a review.) It is well accepted that GABA_{B} receptor agonists and antagonists are pharmacologically useful. For example, the GABA_{B} receptor agonist (−)baclofen, a structural analog of GABA, is a clinically effective muscle relaxant (Bowery & Pratt, 1992, Arzneim.-Forsch./Drug Res. 42:215-223). (−)baclofen, as part of a racemic mixture with (+)baclofen, has been sold in the United States as a muscle relaxant under the name LIORESAL® since 1972.

GABA_{B} receptors represent a large family of related proteins, new family members of which are still being discovered. For example, Kaupmann et al., 1997, Nature 386:239-246 (Kaupmann) reported the cloning and expression of two members of the rat GABA_{B} receptor family, GABA_{B}R1a and GABA_{B}R1b. A variety of experiments using known agonists and antagonists of GABA_{B} receptors seemed to indicate that GABA_{B}R1a and GABA_{B}R1b represented rat GABA_{B} receptors. This conclusion was based primarily on the ability of GABA_{B}R1a and GABA_{B}R1b to bind agonists and antagonist of GABA_{B} receptors with the expected rank order, based upon studies of rat cerebral cortex GABA_{B} receptors. However, there were data that did not fit the theory that Kaupmann had cloned the pharmacologically and functionally active GABA_{B} receptor. For example, Kaupmann noted that agonists had significantly lower binding affinity to recombinant GABA_{B}R1a and GABA_{B}R1b as opposed to native GABA_{B} receptors. Also, Couve et al., 1998, J. Biol. Chem. 273:26361-26367 showed that recombinantly expressed GABA_{B}R1a and GABA_{B}R1b failed to target correctly to the plasma membrane and failed to give rise to functional GABA_{B} receptors when expressed in a variety of cell types.

Examination of the amino acid and gene sequence of GABA_{B}R1a led Kaupmann to propose a structure for GABA_{B}R1a similar to that of the metabotropic glutamate receptor gene family. The metabotropic glutamate receptor family comprises eight glutamate binding receptors and five calcium sensing receptors which exhibit a signal peptide sequence followed by a large N-terminal domain believed to represent the ligand binding pocket that precedes seven transmembrane spanning domains. The hallmark seven transmembrane spanning domains are typical of G-protein coupled receptors (GPCRs), although metabotropic glutamate receptors and GABA_{B}R1a are considerably larger than most GPCRs and contain a signal peptide sequence. No significant amino acid sequence similarities were found between GABA_{B}R1a and GABA_{A} receptors, GABA_{C} receptors, or other typical GPCRs.

Despite work such as that of Kaupmann, pharmacological and physiological evidence indicates that a large number of amino acid binding GABA_{B} receptors remain to be cloned and expressed in recombinant systems where agonists and antagonists can be efficiently identified. In particular, it would be extremely valuable to be able to recombinantly express GABA_{B} receptors in such a manner that not only pharmacologically relevant ligand binding properties would be-exhibited by the recombinant receptors, but also such that the recombinant receptors would show proper functional activity.

### SUMMARY OF THE INVENTION

The present invention is directed to a novel human DNA that encodes a GABA_{B} receptor subunit, HG20. The DNA encoding HG20 is substantially free from other nucleic acids and has the nucleotide sequence shown in SEQ.ID.NO.:1. Also provided is an HG20 protein encoded by the novel DNA sequence. The HG20 protein is substantially free from other proteins and has the amino acid sequence shown in SEQ.ID.NO.:2. Methods of expressing HG20 in recombinant systems and of identifying agonists and antagonists of HG20 are provided.

The present invention is also directed to a novel murine DNA that encodes a GABA_{B} receptor subunit, GABA_{B}R1a. The DNA encoding GABA_{B}R1a is substantially free from other nucleic acids and has the nucleotide sequence shown in SEQ.ID.NO.:19. Also provided is a GABA_{B}R1a protein encoded by the novel DNA sequence. The GABA_{B}R1a protein is substantially free from other proteins and has the amino acid sequence shown in SEQ.ID.NO.:20. Methods of expressing GABA_{B}R1a in recombinant systems and of identifying agonists and antagonists of HG20 are provided.

Also provided by the present invention are methods of co-expressing HG20 and GABA_{B}R1a in the same cells. Such co-expression results in the production of a GABA_{B} receptor that exhibits expected functional properties of GABA_{B} receptors as well as expected ligand binding properties. Recombinant cells co-expressing HG20 and GABA_{B}R1a are provided as well as methods of utilizing such recombinant cells to identify agonists and antagonists of GABA_{B} receptors.

### BRIEF DESCRIPTION OF THE DRAWINGS

FIG. 1A-B shows the complete cDNA sequence of HG20 (SEQ.ID.NO.:1).

FIG. 2 shows the complete amino acid sequence of HG20 (SEQ.ID.NO.:2).

FIG. 3A-B shows predicted signal peptide cleavage sites of HG20. All sequences shown are portions of SEQ.ID.NO.:2.

FIG. 4 shows in situ analysis of the expression of HG20 RNA in squirrel monkey brain.

FIG. 5A shows in vitro coupled transcription/translation of a chimeric FLAG epitope-HG20 (amino acids 52-941) protein.

FIG. 5B shows the expression in COS-7 cells and melanophores of a chimeric FLAG epitope-HG20 (amino acids 52-941) protein.

FIG. 6 shows a comparison of the amino acid sequences of a portion of the N-terminus of HG20 protein and the ligand binding domain of the Pseudomonas aeruginosa amino acid binding protein LIVAT-BP (Swiss Protein database accession number P21175). The upper sequence shown is from HG20 and corresponds to amino acids 63-259 of SEQ.ID.NO.:2. The lower sequence shown is from Pseudomonas aeruginosa LIVAT-BP and is SEQ.ID.NO.:16.

FIG. 7 shows expression in mammalian cells of a chimeric HG20 protein.

FIG. 8 shows a comparison of the amino acid sequences of HG20 and GABA_{B}R1b. The HG20 sequence is SEQ.ID.NO.:2. The GABA_{B}R1b sequence is SEQ.ID.NO.:17.

FIG. 9 shows the expression of recombinant GABA_{B}R1a and HG20 in COS-7 cells. Lanes 1 and 2 show [¹²⁵I]CGP71872 photolabeling of recombinant murine GABA_{B}R1a monomer and dimer in the presence (+) and absence (−) of 1 μM unlabeled CGP71872. Lanes 3 and 4 show that GABA_{B}R1a antibodies 1713.1-1713.2 confirmed (+) expression of recombinantly expressed murine GABA_{B}R1a (referred to as mgb1a here) and absence (−) in pcDNA3.1 mock transfected cells. Lanes 5 and 6 show [¹²⁵I]CGP71872 photolabeling of human FLAG-HG20 in the presence (+) and absence (−) of 1 μM unlabeled CGP71872. Lanes 7 and 8 show that an anti-FLAG antibody confirmed (+) the expression of FLAG-HG20 (referred to as FLAG-gb2 here) and its absence (−) in pcDNA3.1 mock transfected cells. Experimental details were as in Examples 7-9 and 20 except that COS-7 rather than COS-1 cells were used.

FIG. 10 shows co-localization of mRNA for HG20 and GABA_{B}R1a by in situ hybridization histochemistry in rat parietal cortex. Adjacent coronal sections of rat brain showing parietal cortex hybridized with radiolabelled GABA_{B}R1a (A) and HG20 (B) probes. Rat GABA_{B}R1a and HG20 probes were labelled using ³⁵S-UTP (A, B, and D), and autoradiograms were developed after 4 weeks. For co-localization studies, the rat GABA_{B}R1a probe was digoxigenin labelled and developed using anti-digoxigenin HRP, the TSA amplification method and biotinyl tyramide followed by streptavidin-conjugated CY3 (C). (D) shows autoradiography of the same field as in (C), denoting hybridization to HG20 mRNA. (E) is an overlay of images (C) and (D). Arrows denote some of the double-labelled cells. Scale bar=0.5 mm in (A) and (B); scale bar=50 um in (C-E).

FIG. 11 shows functional complementation following co-expression of GABA_{B}R1a and HG20 in Xenopus melanophores. GABA mediated a dose-dependent aggregation response in melanophores co-expressing murine GABA_{B}R1a and FLAG-HG20 (▪) that could be blocked with 100 nM (▾) and 1 μM CGP71872 (▴). The response of GABA on mock-transfected cells is shown (●) as well as a control cannabinoid receptor subtype 2 response to HU210 ligand (inset). This experiment is representative of n=4.

FIG. 12 shows GABA_{B} receptor modulation of forskolin-stimulated cAMP synthesis in HEK293 cells. HEK293 cells stably expressing HG20 (hgb2-42) or GABA_{B}R1a (rgb1a-50) were transiently transfected with GABA_{B}R1a and HG20 expression plasmids to examine the effect of receptor co-expression on modulation of cAMP synthesis. All transfected cells were tested with 300 μM baclofen or GABA (with 100 μM AOAA and 100 μM nipecotic acid) in the absence of forskolin and 30 μM baclofen or GABA in the presence of 10 μM forskolin. Wild-type HEK293 cells were tested with 250 μM baclofen or 250 μM GABA in the presence of 10 μM forskolin. Data are presented as the percent of total cAMP synthesized in the presence of forskolin only. The data presented are from single representative experiments that have been replicated twice. Fsk, forskolin; B, baclofen; G, GABA with AOAA and nipecotic acid. The two right-most set of bar graphs (labeled “B+Fsk” and “G+Fsk”) show that in cells expressing both GABA_{B}R1a and HG20 (rgb1a-50/hgb2 cells (□) and hgb2-42/rgb1a cells (▪)), baclofen and GABA were able to mediate significant reductions in cAMP levels.

FIG. 13 shows that co-expression of GABA_{B}R1a and HG20 permits inwardly rectifying potassium channel (GIRK or Kir) activation in Xenopus oocytes. (A) Representative current families of Kir 3.1/3.2. Currents were evoked by 500 msec voltage commands from a holding potential of −10 mV, delivered in 20 mV increments from −140 to 60 mV. (B) In a protocol designed to measure the effects of various receptors on Kir currents, oocytes were held at −80 mV (a potential where significant inward current is measured). Expression of GABA_{B}R1a or HG20 alone (with or without Giα1) resulted in no modulation of current after GABA treatment. Co-expression of GABA_{B}R1a and FLAG-HG20 receptors followed by treatment with 100 μM GABA resulted in stimulation of Kir 3.1/3.2. Shown are representative traces from at least three independent experiments under each condition.

FIG. 14 shows immunoblotting of murine GABA_{B}R1a and FLAG-HG20 transiently expressed in COS-7 cells. Digitonin-solubilized and anti-FLAG antibody immunoprecipitated membrane proteins were immunoblotted following SDS-PAGE with GABA_{B}R1a antibodies 1713.1-1713.2. The conditions are as follows: mock pcDNA3.1 vector transfected cells (lane 1), FLAG-HG20 expressing cells (lane 2), murine GABA_{B}R1a expressing cells (lane 3), and cells coexpressing murine GABA_{B}R1a and FLAG-HG20 (lane 4). The immunoreactive band corresponding to the GABA_{B}R1a/HG20 heterodimer as well as a band corresponding to the predicted GABA_{B}R1a monomer are denoted by arrows.

FIG. 15 shows the complete cDNA sequence of murine GABA_{B}R1a (SEQ.ID.NO.:19). The sequence shown has been deposited in GenBank (accession number AF114168).

FIG. 16 shows the complete amino acid sequence of murine GABA_{B}R1a (SEQ.ID.NO.:20). The sequence shown has been deposited in GenBank (accession number AF114168).

FIG. 17A-B shows the results of experiments with N- and C-terminal fragments of murine GABA_{B}R 1a. FIG. 17A shows the results of coupled in vitro transcription/translation reactions; lane 1=blank; lane 2=full-length GABA_{B}R1a; lane 3=N-terminal fragment of GABA_{B}R1a; lane 4=C-terminal fragment of GABA_{B}R1a. FIG. 17B shows the results of [¹²⁵I]CGP71872 photoaffinity labeling; lane 1=N-terminal fragment of GABA_{B}R1a; lane 2=N-terminal fragment of GABA_{B}R1a in the presence of GABA; lane 3=C-terminal fragment of GABA_{B}R1a; lane 4=C-terminal fragment of GABA_{B}R1a in the presence of GABA.

FIG. 18A-B shows the amino acid sequence (FIG. 18A) (SEQ.ID.NO.:21) and nucleotide sequence (FIG. 18B) (SEQ.ID.NO.:22) (GenBank accession number AJ012185) of a human GABA_{B}R1a.

FIG. 19A-B shows the nucleotide sequence (SEQ.ID.NO.:23) (GenBank accession number Y11044) of a human GABA_{B}R1a.

FIG. 20 shows a framework map of chromosome 9. The locations of the HG20 gene (referred to as “GPR 51”), markers, and the HSN-1 locus are indicated.

FIG. 21 shows a hydropathy plot for murine GABA_{B}R1a.

FIG. 22 shows a family tree of genes related to HG20. Abbreviations are as follows: hGB1a=human GABA_{B}R1a; mGB1a=mouse GABA_{B}R 1a; rGB1a=rat GABA_{B}R1a; hGB 1b=human GABA_{B}R1b; rGB1b=rat GABA_{B}R1b; ceGB1b=a C. elegans gene related to mammalian GABA_{B}R1a and GABA_{B}R1b; hGB2=human HG20; ceGB2=a C. elegans gene related to human HG20; MGRDROME=a metabotropic glutamate receptor from. Drosophila melanogaster; MGR2 HUMAN=human metabotropic glutamate receptor 2; MGR3 HUMAN=human metabotropic glutamate receptor 3; MGR6 HUMAN=human metabotropic glutamate receptor 6; MGR4 HUMAN=human metabotropic glutamate receptor 4; MGR7 HUMAN=human metabotropic glutamate receptor 7; MGR8 HUMAN=human metabotropic glutamate receptor 8; MGR1 HUMAN=human metabotropic glutamate receptor 1; MGR5 HUMAN=human metabotropic glutamate receptor 5.

FIG. 23 shows the coiled-coil domains in the C-termini of human GABA_{B}R1a and HG20. The upper sequence is from human GABA_{B}R1a and is positions 886-949 of SEQ.ID.NO.:21. The lower sequence is from HG20 and is positions 756-829 of SEQ.ID.NO.:2.

FIG. 24 shows a comparison of the amino acid sequences of human GABA_{B}R1a (referred to as “Human GABA-B1aR,” SEQ.ID.NO.:21); the proteins encoded by two genes from C. elegans (C. elegans GABA-B 1=SEQ.ID.NO.:42 and C. elegans GABA-B2=SEQ.ID.NO.:43); and HG20) (referred to as “Human GABA-B2,” (SEQ.ID.NO.:2). The C. elegans genes have been predicted from C. elegans DNA sequence alone. ZK180 accession number: U58748 is predicted to be GABA-B2 and Y41G9. Contig99 and Y76F7.Contig73 were obtained from the Sanger C. elegans genomic sequence database and are predicted to be GABA-B1.

FIG. 25A-D shows co-immunoprecipitation of the murine GABA_{B}R1a and FLAG-HG20 receptor subunits and immunoblotting using reciprocal receptor subunit antibodies. Murine GABA_{B}R1a and FLAG-HG20 receptors were expressed individually or co-expressed in COS-7 cells. FIG. 25A shows the results of immunoblotting using an anti-murine GABA_{B}R1a antibody. Immunoblot of the solubilized membranes using murine GABA_{B}R1a antibodies 1713.1-1713.2 shows selective expression of murine GABA_{B}R1a in murine GABA_{B}R1a alone expressing cells (lane 3) and murine GABA_{B}R1a/FLAG-HG20 co-expressing cells (lane 4), but not in mock transfected and FLAG-HG20 alone expressing cells (lanes 1 and 2). Staining of GABA_{B}R1a subunits in co-expressing cells is more intense compared to cells expressing the GABA_{B}R1a subunit alone, suggesting that HG20 subunits facilitate GABA_{B}R1a expression. FIG. 25B shows the results of immunoblotting using an anti-FLAG-HG20 antibody. Immunoblotting of the solubilized membranes using the anti-FLAG-HG20 antibody shows selective expression of FLAG-HG20 subunits in FLAG-HG20 alone expressing cells (lane 6) and murine GABA_{B}R1a/FLAG-HG20 co-expressing cells (lane 8), but not in mock transfected and murine GABA_{B}R1a alone expressing cells (lanes 5 and 7). Staining of HG20 subunits in co-expressing cells is more intense compared to cells expressing the HG20 subunit alone, suggesting that GABA_{B}R1a subunits facilitate HG20 expression. FIG. 25C shows the results of immunoprecipitation with an anti-FLAG-HG20 antibody followed by immunoblotting with an anti-murine GABA_{B}R1a antibody. GABA_{B}R1a/HG20 heterodimers are observed only in murine GABA_{B}R1a/FLAG-HG20 co-expressing cells due to the fact that the GABA_{B}R1a subunit was co-immunoprecipitated with the FLAG-HG20 subunit using the FLAG antibody and detected with GABA_{B}R1a antibodies (lane 12). GABA_{B}R1a subunits are not detected in mock-transfected cells and cells expressing GABA_{B}R1a alone or FLAG-HG20 (lanes 9-11). FIG. 25D shows the results of immunoprecipitation with an anti-murine GABA_{B}R1a antibody followed by immunoblotting with an anti-FLAG-HG20 antibody. GABA_{B}R1a/HG20 heterodimers are observed only in murine GABA_{B}R1a/FLAG-HG20 co-expressing cells due to the fact that the FLAG-HG20 subunit was co-immunoprecipitated using the GABA_{B}R1a antibodies and detected with FLAG antibody (lane 16). No FLAG-HG20 subunits are detected in mock-transfected cells or cells expressing murine GABA_{B}R1a alone or FLAG-HG20 (lanes 13-15). The immunoblots shown are from 1-3 independent experiments.

FIG. 26A-B shows some of the motifs in the N-termini of GABA_{B} receptor subunits and related genes. FIG. 26A shows an alignment of murine GABA_{B}R1a (mGABAb1a; a portion of SEQ.ID.NO.:20), human GABA_{B}R1a (hGABAb1a; a portion of SEQ.ID.NO.:21), HG20 (hGABAb2; a portion of SEQ.ID.NO.:2), metabotropic glutamate receptor 1 (mGluR1; SEQ.ID.NO.:44), and two E. coli proteins (LivK (SEQ.ID.NO.:45) and LivBP (SEQ.ID.NO.:46)). FIG. 26B is a schematic drawing showing the location of the various motifs in murine GABA_{B}R1a that are expected to be involved in heterodimer formation of GABA_{B}R1a with HG20.

FIG. 27 shows an expanded view of the coiled-coil region of homology between HG20 (hGABAb2; shown is a portion of SEQ.ID.NO.:2) and murine GABA_{B}R1a (mGABAb1a; a portion of SEQ.ID.NO.:20). Also shown is the corresponding region of human GABA_{B}R1a (hGABAbla; a portion of SEQ.ID.NO.:21).

### DETAILED DESCRIPTION OF THE INVENTION

For the purposes of this invention:

“Substantially free from other proteins” means at least 90%, preferably 95%, more preferably 99%, and even more preferably 99.9%, free of other proteins. Thus, for example, an HG20 protein preparation that is substantially free from other proteins will contain, as a percent of its total protein, no more than 10%, preferably no more than 5%, more preferably no more than 1%, and even more preferably no more than 0.1%, of non-HG20 proteins. Whether a given HG20 protein preparation is substantially free from other proteins can be determined by such conventional techniques of assessing protein purity as, e.g., sodium dodecyl sulfate polyacrylamide gel electrophoresis (SDS-PAGE) combined with appropriate detection methods, e.g., silver staining or immunoblotting.

“Substantially free from other nucleic acids” means at least 90%, preferably 95%, more preferably 99%, and even more preferably 99.9%, free of other nucleic acids. Thus, for example, an HG20 DNA preparation that is substantially free from other nucleic acids will contain, as a percent of its total nucleic acid, no more than 10%, preferably no more than 5%, more preferably no more than 1%, and even more preferably no more than 0.1%, of non-HG20 nucleic acids. Whether a given HG20 DNA preparation is substantially free from other nucleic acids can be determined by such conventional techniques of assessing nucleic acid purity as, e.g., agarose gel electrophoresis combined with appropriate staining methods, e.g., ethidium bromide staining, or by sequencing.

An HG20 polypeptide has “substantially the same biological activity” as native HG20 (i.e., SEQ.ID.NO.:2) if that polypeptide has a K_{d} for a ligand that is no more than 5-fold greater than the K_{d} of native HG20 for the same ligand. An HG20 polypeptide also has “substantially the same biological activity” as HG20 if that polypeptide can form heterodimers with either a GABA_{B}R1a or GABA_{B}R1b polypeptide, thus forming a functional GABA_{B} receptor.

“Functional GABA_{B} receptor” refers to a heterodimer of HG20 and either GABA_{B}R1a or GABA_{B}R1b where the heterodimer displays a functional response when exposed to GABA agonists. Examples of functional responses are: pigment aggregation in Xenopus melanophores, modulation of cAMP levels, coupling to inwardly rectifying potassium channels, mediation of late inhibitory postsynaptic potentials in neurons, increase in potassium conductance, and decrease in calcium conductance. One skilled in the art would be familiar with a variety of methods of measuring the functional responses of G-protein coupled receptors such as the GABA_{B} receptor (see, e.g., Lerner, 1994, Trends Neurosci. 17:142-146 [changes in pigment distribution in melanophore cells]; Yokomizo et al., 1997, Nature 387:620-624 [changes in cAMP or calcium concentration; chemotaxis]; Howard et al., 1996, Science 273:974-977 [changes in membrane currents in Xenopus oocytes]; McKee et al., 1997, Mol. Endocrinol. 11:415-423 [changes in calcium concentration measured using the aequorin assay]; Offermanns & Simon, 1995, J. Biol. Chem. 270:15175, 15180 [changes in inositol phosphate levels]). Depending upon the cells in which heterodimers of HG20 and either GABA_{B}R1a or GABA_{B}R1b are expressed, and thus the G-proteins with which the heterodimers are coupled, certain of such methods may be appropriate for measuring the functional responses of such heterodimers. It is well with the competence of one skilled in the art to select the appropriate method of measuring functional responses for a given experimental system.

A GABA_{B}R1a or GABA_{B}R1b polypeptide has “substantially the same biological activity” as a native GABA_{B}R1a or GABA_{B}R1b polypeptide if that polypeptide has a K_{d} for an amino acid, amino acid analogue, GABA_{B} receptor agonist, or GABA_{B} receptor antagonist such as CGP71872, GABA, saclofen, (−)baclofen, or (L)-glutamic acid that is no more than 5-fold greater than the K_{d} of a native GABA_{B}R1a or GABA_{B}R1b polypeptide for the same amino acid, amino acid analogue, GABA_{B} receptor agonist, or GABA_{B} receptor antagonist. A GABA_{B}R1a or GABA_{B}R1b polypeptide also has “substantially the same biological activity” as a native GABA_{B}R1a or GABA_{B}R1b polypeptide if that polypeptide can form heterodimers with an HG20 polypeptide, thus forming a functional GABA_{B} receptor. Native GABA_{B}R1a or GABA_{B}R1b polypeptides include the murine GABA_{B}R1a sequence shown as SEQ.ID.NO.:20; the rat GABA_{B}R1a or GABA_{B}R1b polypeptides disclosed in Kaupmann et al., 1997, Nature 386:239-246; the human GABA_{B}R1a sequence disclosed in GenBank accession number AJ012185 (SEQ.ID.NO.:21); and the protein encoded by the DNA sequence disclosed in GenBank accession number Y11044 (SEQ.ID.NO.:23).

A “conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutamic acid for aspartic acid).

The present invention relates to the identification and cloning of HG20, a novel G-protein coupled receptor-like protein that represents a subunit for the GABA_{B} receptor. The present invention provides DNA encoding HG20 that is substantially free from other nucleic acids. The present invention also provides recombinant DNA molecules encoding HG20 as well as isolated DNA molecules encoding HG20. Following the cloning of HG20 by the present inventors, a sequence highly similar to the sequence of HG20 was deposited in GenBank by Clark et al. (GenBank accession number AF056085), by White et al. (GenBank accession number AJ012188), and by Borowsky et al. (GenBank accession number AF074483). Two ESTs (GenBank accession number T07621, deposited Jun. 30, 1993, and GenBank accession number Z43654, deposited Sep. 21, 1995) each contain partial sequences of HG20 cDNA.

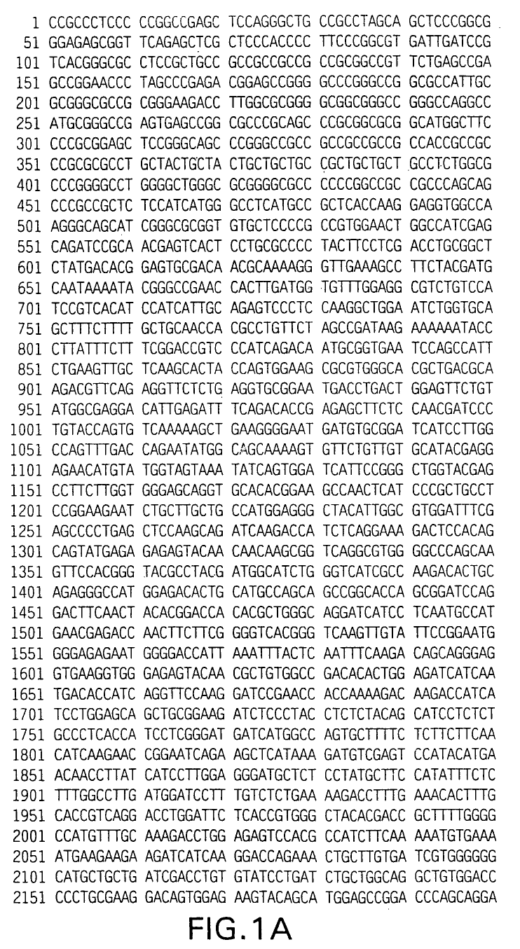

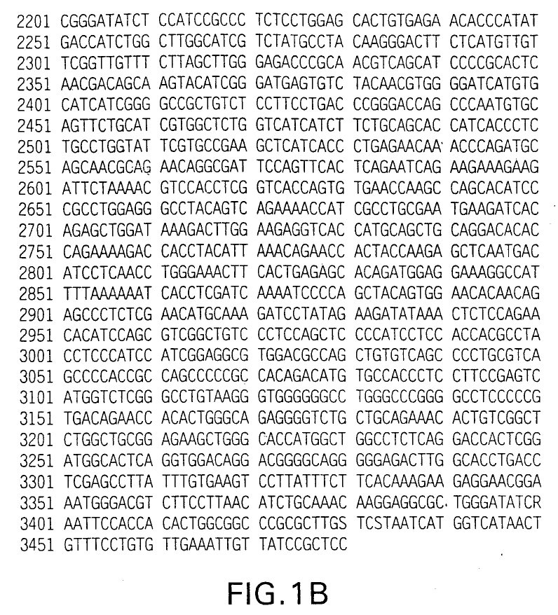

The present invention provides a DNA molecule substantially free from other nucleic acids comprising the nucleotide sequence shown in FIG. 1 as SEQ.ID.NO.:1. Analysis of SEQ.ID.NO.:1 revealed that it contains a long open reading frame at positions 293-3,115. Thus, the present invention also provides a DNA molecule substantially free from other nucleic acids comprising the nucleotide sequence of positions 293-3,115 of SEQ.ID.NO.:1. The present invention also provides an isolated DNA molecule comprising the nucleotide sequence of positions 293-3,115 of SEQ.ID.NO.:1.

Sequence analysis of the open reading frame of the HG20 DNA revealed that it encodes a protein of 941 amino acids with a calculated molecular weight of 104 kd and a predicted signal peptide. The predicted amino acid sequence of HG20 is 36% identical to the metabotropic GABA receptor-like sequence GABA_{B}R1a described in Kaupmann (see above) throughout the entire sequence, and thus HG20 most likely represents a novel metabotropic GABA receptor or receptor subunit. In situ hybridization showed that HG20 RNA is highly expressed in the cortex, thalamus, hippocampus, and cerebellum of the brain, showing overlapping distribution with GABA_{B}R1a RNA as judged by in situ hybridization as well as with the expression of GABA_{B} receptors as judged by pharmacological studies. HG20 RNA exhibits restricted distribution in the periphery, with low abundance of the 6.5 kb RNA in the heart, spleen, and pancreas and high levels in the adrenal gland. HG20 recombinantly expressed in COS-1 cells showed no specific binding for [³H](+)baclofen, and when expressed in Xenopus oocyte and Xenopus melanophore functional assays, showed no activity to GABA, (−)baclofen, and glutamic acid.

The novel DNA sequences of the present invention encoding HG20, in whole or in part, can be linked with other DNA sequences, i.e., DNA sequences to which HG20 is not naturally linked, to form “recombinant DNA molecules” containing HG20. Such other sequences can include DNA sequences that control transcription or translation such as, e.g., translation initiation sequences, promoters for RNA polymerase II, transcription or translation termination sequences, enhancer sequences, sequences that control replication in microorganisms, or that confer antibiotic resistance. The novel DNA sequences of the present invention can be inserted into vectors such as plasmids, cosmids, viral vectors, or yeast artificial chromosomes.

The present invention also includes isolated forms of DNA encoding HG20. By “isolated DNA encoding HG20” is meant DNA encoding HG20 that has been isolated from a natural source or produced by recombinant means. Use of the term “isolated” indicates that DNA encoding HG20 is not present in its normal cellular environment. Thus, an isolated DNA encoding HG20 may be in a cell-free solution or placed in a different cellular environment from that in which it occurs naturally. The term isolated does not imply that isolated DNA encoding HG20 is the only DNA present but instead means that isolated DNA encoding HG20 is at least 95% free of non-nucleic acid material (e.g., proteins, lipids, carbohydrates) naturally associated with the DNA encoding HG20. Thus, DNA encoding HG20 that is expressed in bacteria or even in eukaryotic cells which do not naturally (i.e., without human intervention) contain it through recombinant means is “isolated DNA encoding HG20.”

Included in the present invention are DNA sequences that hybridize to SEQ.ID.NO.:1 under stringent conditions. By way of example, and not limitations a procedure using conditions of high stringency is as follows: Prehybridization of filters containing DNA is carried out for 2 hr. to overnight at 65° C. in buffer composed of 6×SSC, 5× Denhardt's solution, and 100 μg/ml denatured salmon sperm DNA. Filters are hybridized for 12 to 48 hrs at 65° C. in prehybridization mixture containing 100 μg/ml denatured salmon sperm DNA and 5-20×10⁶ cpm of ³²P-labeled probe. Washing of filters is done at 37° C. for 1 hr in a solution containing 2×SSC, 0.1% SDS. This is followed by a wash in 0.1×SSC, 0.1% SDS at 50° C. for 45 min. before autoradiography.

Other procedures using conditions of high stringency would include either a hybridization carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Reagents mentioned in the foregoing procedures for carrying out high stringency hybridization are well known in the art. Details of the composition of these reagents can be found in, e.g., Sambrook, Fritsch, and Maniatis, 1989, Molecular Cloning: A Laboratory Manual, second edition, Cold Spring Harbor Laboratory Press. In addition to the foregoing, other conditions of high stringency which may be used are well known in the art.

Another aspect of the present invention includes host cells that have been engineered to contain and/or express DNA sequences encoding HG20. Such recombinant host cells can be cultured under suitable conditions to produce HG20. An expression vector containing DNA encoding HG20 can be used for expression of HG20 in a recombinant host cell. Recombinant host cells may be prokaryotic or eukaryotic, including but not limited to, bacteria such as E. coli, fungal cells such as yeast, mammalian cells including, but not limited to, cell lines of human, bovine, porcine, monkey and rodent origin, and insect cells including but not limited to Drosophila and silkworm derived cell lines. Cell lines derived from mammalian species which are suitable for recombinant expression of HG20 and which are commercially available, include but are not limited to, L cells L-M(TK⁻) (ATCC CCL 1.3), L cells L-M (ATCC CCL 1.2), HEK293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH3T3 (ATCC CRL 1658), HeLa (ATCC CCL 2), C127I (ATCC CRL 1616), BS-C-1 (ATCC CCL 26), MRC-5 (ATCC CCL 171), Xenopus melanophores, and Xenopus oocytes. In particular embodiments, the recombinant cells expressing HG20 protein co-express a GABA_{B}R1a or GABA_{B}R1b protein, thus forming a functional GABA_{B} receptor comprising a heterodimer of HG20 and either GABA_{B}R1a or GABA_{B}R1b. In particular embodiments, the recombinant cells have been transfected with expression vectors that direct the expression of HG20 and GABA_{B}R1a or GABA_{B}R1b.

Cells that are particularly suitable for expression of the HG20 protein are melanophore pigment cells from Xenopus laevis. Such melanophore pigment cells can be used for functional assays that employ recombinant expression of HG20 in a manner similar to the use of such melanophore pigment cells for the functional assay of other recombinant GPCRs (Graminski et al., 1993, J. Biol. Chem. 268:5957-5964; Lerner, 1994, Trends Neurosci. 17:142-146; Potenza & Lerner, 1992, Pigment Cell Res. 5:372-378; Potenza et al., 1992, Anal. Biochem. 206:315-322). Especially preferred are Xenopus melanophore pigment cells co-expressing HG20 and GABA_{B}R1a or GABA_{B}R1b, in which HG20 has formed a heterodimer with GABA_{B}R1a or GABA_{B}R1b, thus forming a functional GABA_{B} receptor. The presence of functional GABA_{B} receptors in such cells can be determined by the use of assays such as the pigment aggregation assay described herein. Other assays that reflect a decrease in cAMP levels mediated by exposure to GABA or other agonists of GABA_{B} receptors would also be suitable.

Also preferred are stably or transiently transfected HEK293 cells co-expressing HG20 and GABA_{B}R1a or GABA_{B}R1b, in which HG20 has formed a heterodimer with GABA_{B}R1a or GABA_{B}R1b, thus forming a functional GABA_{B} receptor. The presence of functional GABA_{B} receptors in such cells can be determined by the use of assays such as those that measure cAMP levels as described herein.

Also preferred are Xenopus oocytes co-expressing HG20 and GABA_{B}R1a or GABA_{B}R1b, in which HG20 has formed a heterodimer with GABA_{B}R1a or GABA_{B}R1b, thus forming a functional GABA_{B} receptor. The presence of functional GABA_{B} receptors in such cells can be determined by the use of assays that measure coupling of functional GABA_{B} receptors comprising heterodimers of HG20 and GABA_{B}R1a or GABA_{B}R1b to inwardly rectifying potassium channels (especially the Kir3 family).

In order to produce the above-described cells co-expressing HG20 and GABA_{B}R1a or GABA_{B}R1b, expression vectors comprising DNA encoding HG20 and GABA_{B}R1a or GABA_{B}R1b can be transfected into the cells. HG20 and GABA_{B}R1a or GABA_{B}R1b can be transfected separately, each on its own expression vector, or, alternatively, a single expression vector encoding both HG20 and GABA_{B}R1a or GABA_{B}R1b can be used.

A variety of mammalian expression vectors can be used to express recombinant HG20, GABA_{B}R1a or GABA_{B}R1b in mammalian cells. Commercially available mammalian expression vectors which are suitable include, but are not limited to, pMC1neo (Stratagene), pSG5 (Stratagene), pcDNAI and pcDNAIamp, pcDNA3, pcDNA3.1, pCR3.1 (Invitrogen), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), and the PT7TS oocyte expression vector (or similar expression vectors containing the globin 5′ UTR and the globin 3′ UTR). The choice of vector will depend upon cell type used, level of expression desired, and the like. Following expression in recombinant cells, HG20, GABA_{B}R1a GABA_{B}R1b, or heterodimers of HG20 and either GABA_{B}R1a or GABA_{B}R1b can be purified to a level that is substantially free from other proteins by conventional techniques, e.g., salt fractionation, ion exchange chromatography, size exclusion chromatography, hydroxylapatite adsorption chromatography, hydrophobic interaction chromatography, and preparative gel electrophoresis. Also, membrane preparations comprising HG20, GABA_{B}R1a GABA_{B}R1b, or heterodimers of HG20 and either GABA_{B}R1a or GABA_{B}R1b can be prepared. Especially preferred are membrane preparations that comprise heterodimers of HG20 and either GABA_{B}R1a or GABA_{B}R1b in which the heterodimers represent functional GABA_{B} receptors.

The present invention includes a method of producing HG20 protein comprising:
(a) transfecting a host cell with an expression vector comprising DNA that encodes an HG20 protein;
(b) growing the host cells under conditions such that HG20 protein is produced; and
(c) recovering HG20 protein from the host cells.

In particular embodiments, the method of recovering HG20 protein involves obtaining membrane preparations that contain HG20 protein from the host cells. In particular embodiments, such membrane preparations contain heterodimers of HG20 protein and GABA_{B}R1a or GABA_{B}R1b protein that form functional GABA_{B} receptors.

The present invention includes a method of expressing a truncated HG20 protein comprising:
(a) transfecting a host cell with an expression vector comprising DNA that encodes an HG20 protein that has been truncated at the amino or carboxyl terminus;
(b) culturing the transfected cells of step (a) under conditions such that the truncated HG20 protein is expressed.

Truncated HG20 proteins are those HG20 proteins in which contiguous portions of the N terminus or C terminus have been removed. For example, positions 52-941 of SEQ.ID.NO.:2 represents a truncated HG20 protein. Truncated HG20 proteins may be fused in frame to non-HG20 amino acid sequences, as, e.g., in the FLAG-HG20 construct described herein.

The present invention includes a method of producing functional GABA_{B} receptors in cells comprising:
(a) transfecting cells with:
   (1) an expression vector that directs the expression of HG20 in the cells; and
   (2) an expression vector that directs the expression of GABA_{B}R1a or GABA_{B}R1b in the cells;
(b) culturing the cells under conditions such that heterodimers of HG20 and GABA_{B}R1a or GABA_{B}R1b are formed where the heterodimers constitute functional GABA_{B} receptors.

In particular embodiments of the above methods, the cells are eukaryotic cells. In other embodiments, the cells are mammalian cells. In still other embodiments, the cells are COS cells, e.g., COS-7 cells (ATCC CRL 1651) or COS-1 cells (ATCC CRL 1650); HEK293 cells (ATCC CRL 1573); or Xenopus melanophores.

In particular embodiments, the HG20 protein comprises the amino acid sequence shown in SEQ.ID.NO.:2. In particular embodiments, the HG20 protein is a truncated HG20 protein. In particular embodiments, the truncated HG20 protein comprises amino acids 52-941 of SEQ.ID.NO.:2. In particular embodiments, the truncated HG20 protein is a chimeric HG20 protein.

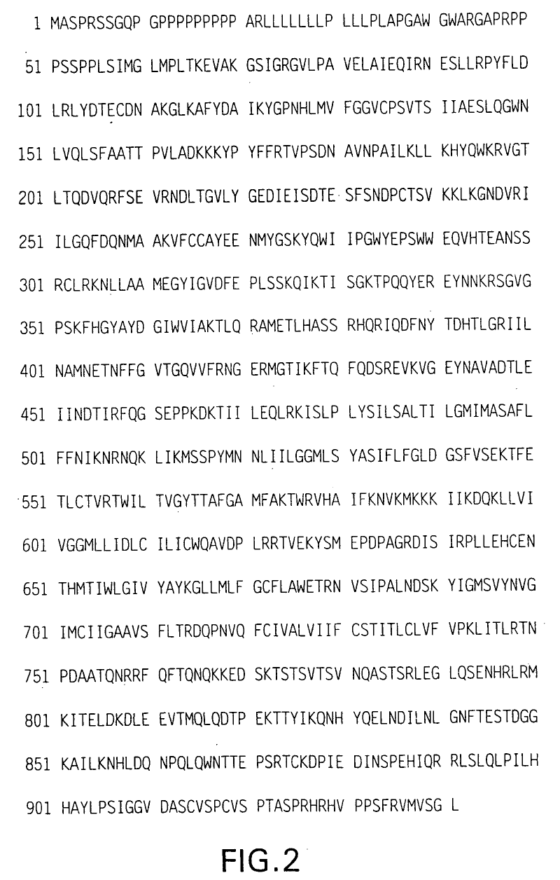

The present invention includes HG20 protein substantially free from other proteins. The amino acid sequence of the full-length HG20 protein is shown in FIG. 2 as SEQ.ID.NO.:2. Thus, the present invention includes polypeptides comprising HG20 protein substantially free from other proteins where the polypeptides comprise the amino acid sequence SEQ.ID.NO.:2. The present invention also includes polypeptides comprising HG20 proteins lacking a signal sequence. Examples of amino acid sequences of HG20 proteins lacking a signal sequence are:
Positions 9-941 of SEQ.ID.NO.:2;
Positions 35-941 of SEQ.ID.NO.:2;
Positions 36-941 of SEQ.ID.NO.:2;
Positions 38-941 of SEQ.ID.NO.:2;
Positions 39-941 of SEQ.ID.NO.:2;
Positions 42-941 of SEQ.ID.NO.:2;
Positions 44-941 of SEQ.ID.NO.:2;
Positions 46-941 of SEQ.ID.NO.:2;
Positions 52-941 of SEQ.ID.NO.:2; and
Positions 57-941 of SEQ.ID.NO.:2.

The present invention also includes DNA encoding the above-described HG20 proteins lacking a signal sequence. Thus, e.g., the present invention includes a DNA molecule comprising a nucleotide sequence selected from the group consisting of:
Positions 293-3,115 of SEQ.ID.NO.:1;
Positions 317-3,115 of SEQ.ID.NO.:1;
Positions 395-3,115 of SEQ.ID.NO.:1;
Positions 398-3,115 of SEQ.ID.NO.:1;
Positions 404-3,115 of SEQ.ID.NO.:1;
Positions 407-3,115 of SEQ.ID.NO.:1;
Positions 416-3,115 of SEQ.ID.NO.:1;
Positions 422-3,115 of SEQ.ID.NO.:1;
Positions 428-3,115 of SEQ.ID.NO.:1;
Positions 446-3,115 of SEQ.ID.NO.:1; and
Positions 461-3,115 of SEQ.ID.NO.:1.

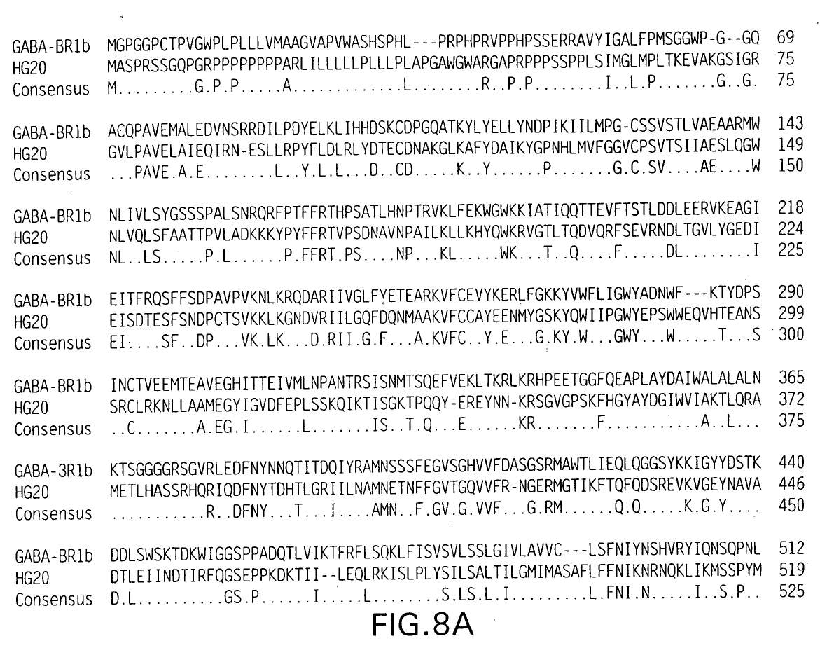

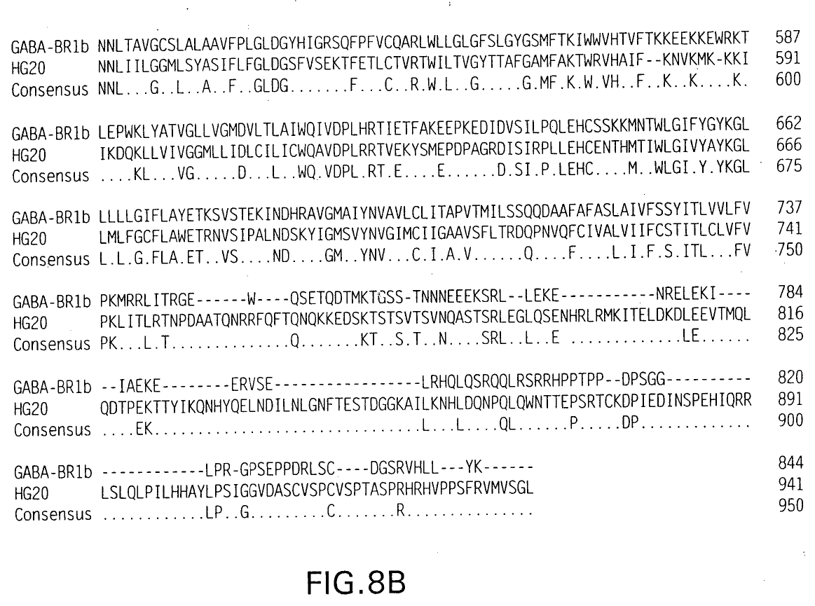

As with many receptor proteins, it is possible to modify many of the amino acids of HG20, particularly those which are not found in the ligand binding domain, and still retain substantially the same biological activity as the original protein. Thus this invention includes modified HG20 polypeptides which have amino acid deletions, additions, or substitutions but that still retain substantially the same biological activity as native HG20. It is generally accepted that single amino acid substitutions do not usually alter the biological activity of a protein (see, e.g., Molecular Biology of the Gene, Watson et al., 1987, Fourth Ed., The Benjamin/Cummings Publishing Co., Inc., page 226; and Cunningham & Wells, 1989, Science 244:1081-1085). Accordingly, the present invention includes polypeptides where one amino acid substitution has been made in SEQ.ID.NO.:2 or in one of the HG20 polypeptides lacking a signal sequence listed above, wherein the polypeptides still retain substantially the same biological activity as native HG20. The present invention also includes polypeptides where two or more amino acid substitutions have been made in SEQ.ID.NO.:2 or in one of the HG20 polypeptides lacking a signal sequence listed above, wherein the polypeptides still retain substantially the same biological activity as native HG20. In particular, the present invention includes embodiments where the above-described substitutions are conservative substitutions. In particular, the present invention includes embodiments where the above-described substitutions do not occur in the ligand-binding domain of HG20. In particular, the present invention includes embodiments where amino acid changes have been made in the positions of HG20 where the amino acid sequence of HG20 differs from the amino acid sequence of GABA_{B}R1b (see FIG. 8).

The present invention also includes C-terminal truncated forms of HG20, particularly those which encompass the extracellular portion of the receptor, but lack the intracellular signaling portion of the receptor. Such truncated receptors are useful in various binding assays described herein, for crystallization studies, and for structure-activity-relationship studies. Accordingly, the present invention includes an HG20 protein substantially free from other proteins having the amino acid sequence of positions 1-480 of SEQ.ID.NO.:2.

O'Hara et al., 1993, Neuron 11:41-52 (O'Hara) reported that the amino terminal domains of several metabotropic glutamate receptors showed amino acid sequence similarities to the amino termini of several bacterial periplasmic binding proteins. O'Hara used this similarity to predict, and then experimentally confirm, that these amino terminal domains correspond to the location of the ligand binding domains of these metabotropic glutamate receptors.

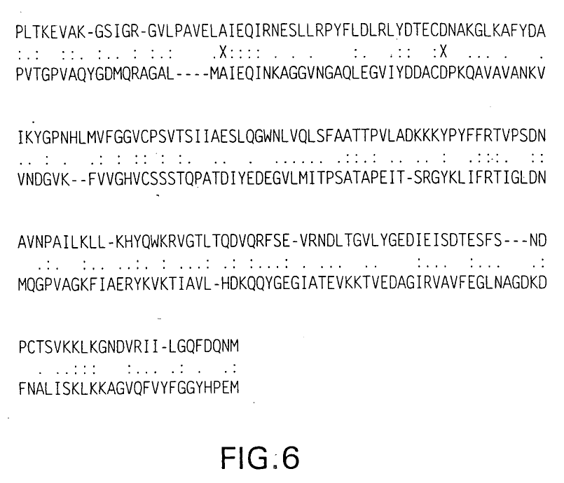

The present inventors have discovered a region of amino acid sequence in the N-terminal domain of HG20 that is similar to the amino acid sequence of the bacterial periplasmic binding protein Leucine, Isoleucine, Valine (Alanine and Threonine) Binding Protein (LIVAT-BP) of Pseudomonas aeruginosa. See FIG. 6. The region shown is about 25% identical between the two proteins. This is above the maximum identity of 17% reported by O'Hara between any one metabotropic glutamate receptor and any one periplasmic binding protein and indicates that the region of HG20 depicted is highly likely to contain the ligand binding domain.

Accordingly, the present invention includes a polypeptide representing the ligand binding domain of HG29 that includes amino acids 63-259 of SEQ.ID.NO.:2. Also provided are chimeric proteins comprising amino acids 63-259 of SEQ.ID.NO.:2.

Romano et al., 1996, J. Biol. Chem. 271:28612-28616 demonstrated that metabotropic glutamate receptors are often found as homodimers formed by an intermolecular disulfide bond. The location of the cysteines responsible for the disulfide bond was found to be in the amino terminal 17 kD of the receptors. Transmembrane interactions may also contribute to functional GABA_{B} receptor dimer formation, as previously reported for the dopamine D2 receptor and, β2-adrenergic receptor (Ng et al., 1996, Biochem. Biophys. Res. Comm. 227:200-204; Hebert et al., 1996, J. Biol. Chem. 271, 16384-16392). Accordingly, the present invention includes dimers of HG20 proteins. In particular embodiments, the HG20 protein has an amino acid selected from the group consisting of:
SEQ.ID.NO.:2;
Positions 9-941 of SEQ.ID.NO.:2;
Positions 35-941 of SEQ.ID.NO.:2;
Positions 36-941 of SEQ.ID.NO.:2;
Positions 38-941 of SEQ.ID.NO.:2;
Positions 39-941 of SEQ.ID.NO.:2;
Positions 42-941 of SEQ.ID.NO.:2;
Positions 44-941 of SEQ.ID.NO.:2;
Positions 46-941 of SEQ.ID.NO.:2;
Positions 52-941 of SEQ.ID.NO.:2;
Positions 57-941 of SEQ.ID.NO.:2; and
Positions 1-480 of SEQ.ID.NO.:2.

It has been found that, in some cases, membrane spanning regions of receptor proteins can be used to inhibit receptor function (Ng et al., 1996, Biochem. Biophys. Res. Comm. 227:200-204; Hebert et al., 1996, J. Biol. Chem. 271, 16384-16392; Lofts et al., Oncogene 8:2813-2820). Accordingly, the present invention provides peptides derived from the seven membrane spanning regions of HG20 and their use to inhibit HG20 or GABA_{B} receptor function. Such peptides can include the whole or parts of the membrane spanning domains.

The present invention also includes isolated forms of HG20 proteins. By “isolated HG20 protein” is meant HG20 protein that has been isolated from a natural source or produced by recombinant means. Use of the term “isolated” indicates that HG20 protein is not present in its normal cellular environment. Thus, an isolated HG20 protein may be in a cell-free solution or placed in a different cellular environment from that in which it occurs naturally. The term isolated does not imply that an isolated HG20 protein is the only protein present but instead means that an isolated HG20 protein is at least 95% free of non-amino acid material (e.g., nucleic acids, lipids, carbohydrates) naturally associated with the HG20 protein. Thus, an HG20 protein that is expressed through recombinant means in bacteria or even in eukaryotic cells which do not naturally (i.e., without human intervention) express it is an “isolated HG20 protein.”

The present invention also includes chimeric HG20 proteins. By chimeric HG20 protein is meant a contiguous polypeptide sequence of HG20 fused in frame to a polypeptide sequence of a non-HG20 protein. For example, the N-terminal domain and seven transmembrane spanning domains of HG20 fused at the C-terminus in frame to a G protein would be a chimeric HG20 protein. Another example of a chimeric HG20 protein would be a polypeptide comprising the FLAG epitope fused in frame at the amino terminus of amino acids 52-941 of SEQ.ID.NO.:2.

The present invention also includes HG20 proteins that are in the form of multimeric structures, e.g., dimers. Such multimers of other metabotropic G-protein coupled receptors are known (Hebert et al., 1996, J. Biol. Chem. 271, 16384-16392; Ng et al., 1996, Biochem. Biophys. Res. Comm. 227, 200-204; Romano et al., 1996, J. Biol. Chem. 271, 28612-28616).

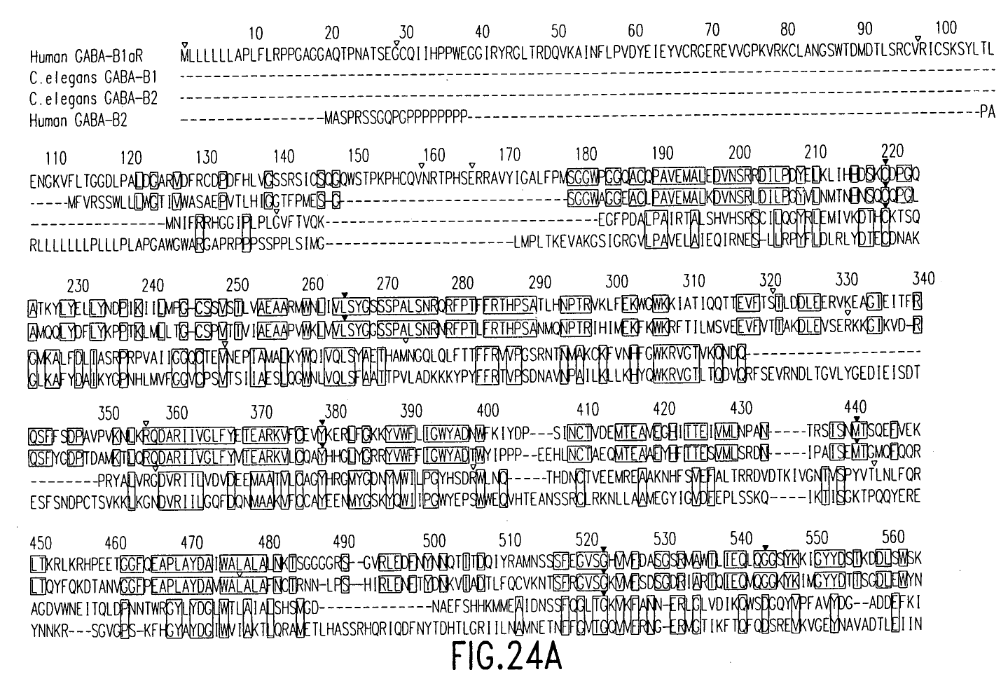

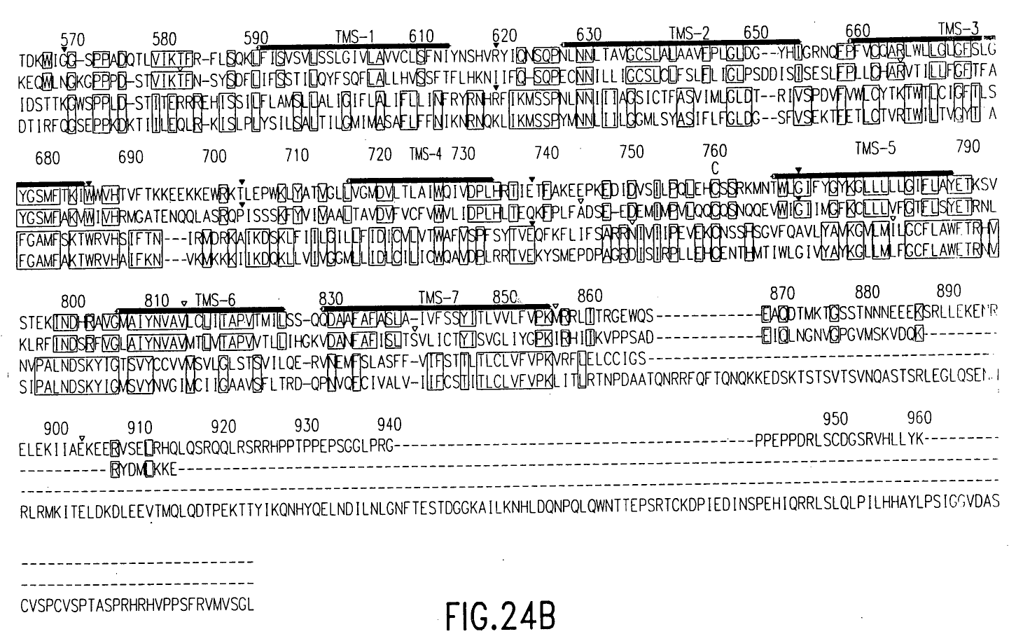

Preferred forms of dimers of HG20 are heterodimers comprising HG20 and other G-protein coupled receptors (GPCRs). Such GPCRs could be, e.g., other subunits of GABA_{B} receptors, proteins from C. elegans showing homology to HG20 (see FIG. 24), or human GPCRs that are homologs of the C. elegans proteins. Particularly preferred forms of heterodimers are heterodimers of HG20 and either GABA_{B}R1a or GABA_{B}R1b. It has been found by the present inventors that such heterodimers exhibit functional properties of GABA_{B} receptors while monomers or homodimers of HG20, GABA_{B}R1a, or GABA_{B}R1b do not exhibit functional properties. Another likely heterodimer partner for HG20 is the protein corresponding to the sequence deposited in GenBank at accession number 3776096.

The strongest evidence that functional GABA_{B} receptors require both HG20 and GABA_{B}R1a or GABA_{B}R1b comes from studies demonstrating that co-transfection and co-expression of both HG20 and either GABA_{B}R1a or GABA_{B}R1b is necessary in order for the detection of GABA_{B} receptor functional responses. Transfection and expression of HG20, GABA_{B}R1a or GABA_{B}R1b alone does not lead to the production of functional GABA_{B} receptors.

For example, in Xenopus melanophores co-expressing HG20 and GABA_{B}R1a but not in melanophores expressing HG20 or GABA_{B}R1a alone, or in mock transfected melanophores, GABA mediated a dose-dependent pigment aggregation response that could be inhibited with the GABA_{B} receptor specific CGP71872 antagonist. This pigment aggregation response is associated with a decrease in intracellular cAMP levels. Such a decrease has been confirmed in HEK293 cells. Also, co-expression of HG20 and GABA_{B}R1a in Xenopus oocytes resulted in the stimulation of inwardly rectifying potassium currents (Kirs). Native functional GABA_{B} receptors have been reported to couple to Kirs (Misgeld et al., 1995, Prog. Neurobiol. 46:423-462).

Consistent with the need for both HG20 and GABA_{B}R1a for detection of functional GABA_{B} receptors in transfected cells, the present inventors have demonstrated that HG20 and GABA_{B}R1a form heterodimers by immunoprecipitation of HG20 followed by immunoblotting with a GABA_{B}R1a antibody.

That a functional GABA_{B} receptor requires both HG20 and either GABA_{B}R1a or GABA_{B}R1b is also suggested by the observation that GABA_{B}R1a or GABA_{B}R1b, recombinantly expressed in the absence of HG20, binds ligand with much reduced affinity compared to the affinity of native GABA_{B} receptors. Also, characterization of the tissue distribution of each of the receptors by in situ hybridization histochemistry in rat brain revealed co-localization of HG20 and GABA_{B}R1a transcripts in many brain regions, including cortex, at both the regional and cellular levels.

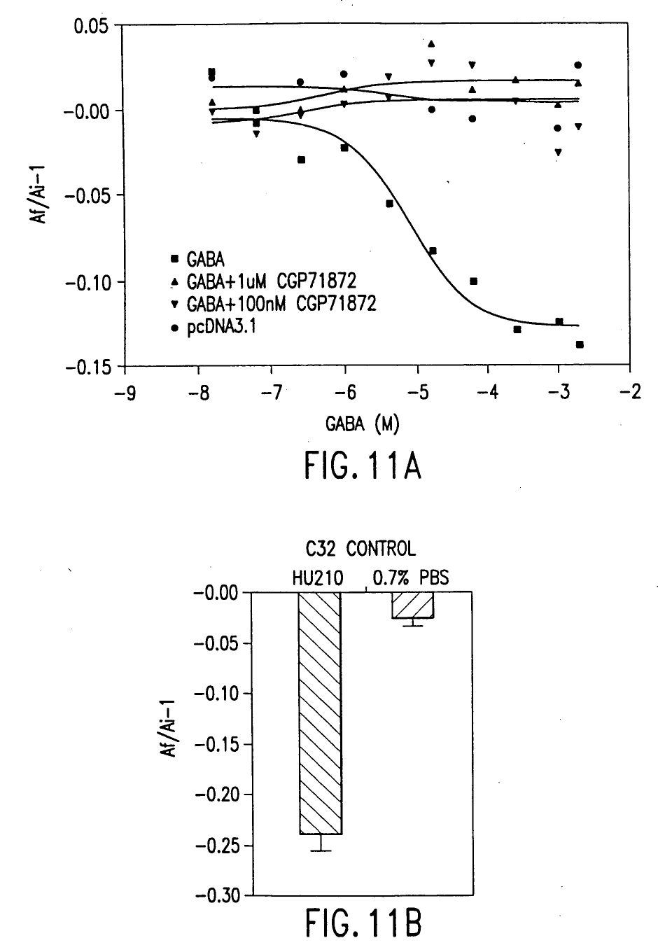

The Xenopus melanophore pigment aggregation/dispersion assay has been shown to be highly suitable for monitoring agonist activation of Gi-, Gq-, and Gs-coupled receptors (Potenza et al., 1992, Anal. Biochem. 206:315-322; Lerner, 1994, Trends Neurosci. 17:142-146. Agonist activation of Gi-coupled receptors expressed in melanophores results in pigment aggregation via a reduction in intracellular cAMP levels, whereas activation of Gs- and Gq-coupled receptors results in pigment dispersion via elevations in intracellular cAMP and calcium levels, respectively. Melanophores transfected separately with either GABA_{B}R1a or HG20 showed no pigment aggregation or dispersion response following treatment with up to 1 mM concentrations of (L)-glutamic acid, GABA, or prototypic GABAergic agonists: (−)baclofen, 3-aminopropyl-(methyl)phosphonic acid, cis-4-aminocrotonic acid, piperidine-4-sulfonic acid (data not shown). Similarly, both receptors failed to couple to K⁺ channels in Xenopus oocytes under patch-clamp conditions when transfected separately (data not shown). However, in melanophores transiently co-transfected with GABA_{B}R1a and HG20, GABA mediated a dose-dependent aggregation response with an IC₅₀ value of 3-7 μM (n=3). This aggregation was absent in mock-transfected cells and in cells transfected with GABA_{B}R1a or HG20 alone (FIG. 11). The GABA-mediated activity represented 42-56% (n=3) of a control cannabinoid receptor subtype 2 response (FIG. 11, inset), and could be inhibited by the CGP71872 antagonist (n=3), indicating it was GABA_{B} receptor specific (FIG. 11). GABA_{B}R1a was expressed by subcloning full-length GABA_{B}R1a into the NheI-NotI site of pcDNA3.1 or pCIneo; HG20 was expressed as a FLAG-HG20 chimeric protein. See Examples 11 and 20 for further experimental details of expression vectors used, transfection conditions, assay conditions, etc. for the above-described co-expression studies.

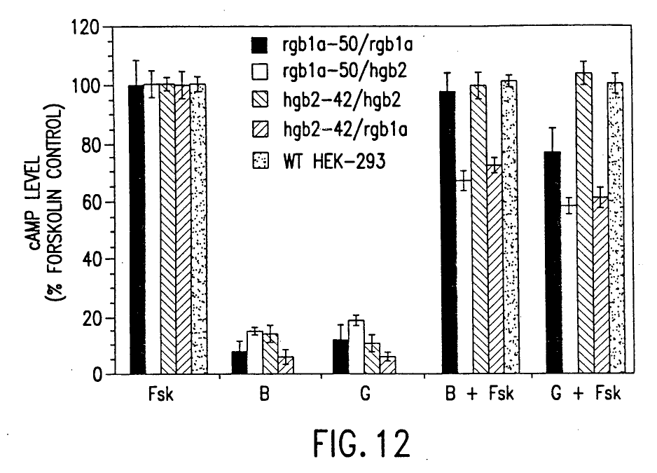

The functional data arising from co-expression of GABA_{B}R1a and HG20 receptors were confirmed in HEK293 cells. HEK293 cells transfected with and stably expressing GABA_{B}R1a and HG20 were selected based on expression of receptor message as determined by dot blot analyses. In cell lines stably expressing the individual receptors, we observed small and inconsistent responses in assays to examine agonist-mediated modulation of cAMP synthesis. However, transient transfection of HEK293 cells stably expressing GABA_{B}R1a (rgb1a-50) with an HG20 expression plasmid and transient transfection of HEK293 cells stably expressing HG20 (hgb2-42) with a GABA_{B}R1a expression plasmid significantly enhanced the ability of baclofen and GABA to inhibit forskolin-stimulated cAMP synthesis. Rgb1a-50 cells transfected with HG20 exhibited a 28% reduction in forskolin-stimulated cAMP synthesis with 30 μM baclofen and a 40% decrease with 30 μM GABA plus 100 μM aminocxyacetic acid (AOAA; a GABA transaminase inhibitor) and 100 μM nipecotic acid (a GABA uptake inhibitor) (FIG. 12B). A 34% reduction in forskolin-stimulated cAMP synthesis was observed for hgb2-42 cells transfected with GABA_{B}R1a treated with baclofen and a 43% decrease was observed for GABA plus AOAA and nipecotic acid (FIG. 12B). While inhibition of cAMP synthesis was sometimes observed with rgb1a-50 cells transfected with GABA_{B}R1a and hgb2-42 cells transfected with HG20, these effects were small and inconsistent (0-20% inhibition; FIG. 12B). Neither baclofen nor GABA plus AOAA and nipecotic acid in the absence of forskolin had any affect on cAMP synthesis (FIG. 12B). In addition, wild-type HEK293 cells did not exhibit baclofen- or GABA-mediated inhibition of forskolin-stimulated cAMP synthesis (FIG. 12B). These data demonstrate that the functional GABA_{B} receptor requires both GABA_{B}R1a and HG20. For experimental details of these studies in HEK293 cells, see Example 12.

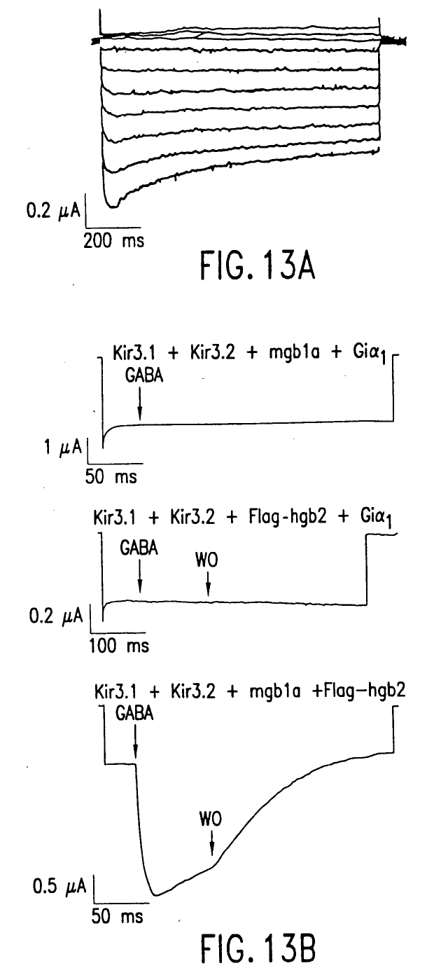

Co-expression of the GABA_{B}R1a and HG20 with the inwardly rectifying potassium channels Kir 3.1/3.2 in Xenopus oocytes resulted in a significant stimulation of inwardly rectifying potassium current (Kir) in response to GABA [301 +/−20.6%, (n=3) increase over control current] measured at −80 mV which could subsequently be washed out with control solution (FIG. 13). Modulation of Kir 3.1/3.2 was not seen in oocytes expressing GABA_{B}R1a or HG20 individually, even in the presence of Giα1 (FIG. 13). See Example 21 for details.

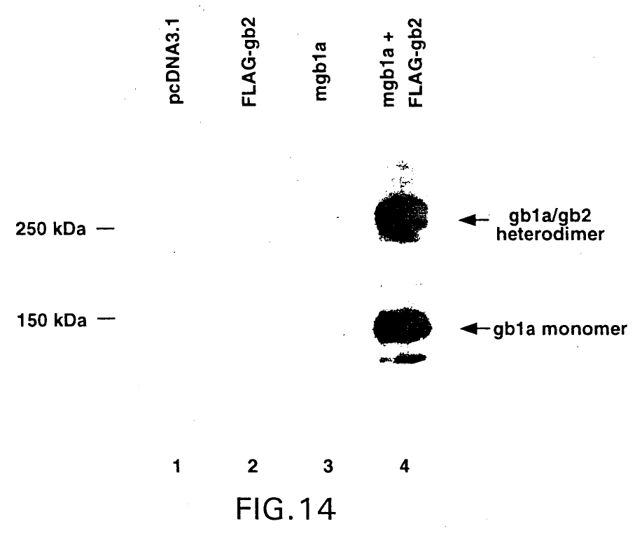

To determine whether receptor intermolecular interactions accounted for the functional activity that was observed following the co-expression of recombinant GABA_{B}R1a and HG20, membranes from cells co-expressing GABA_{B}R1a and HG20 or the individual proteins were first immunoprecipitated using anti-FLAG antibodies (to detect the recombinant FLAG-HG20 chimeric proteins) followed by immunoblotting with a GABA_{B}R1a-specific antibody. As seen in FIG. 14, lanes 1-3, no GABA_{B}R1a immunoreactivity was detected in samples prepared from mock vector transfected cells, FLAG-HG20 alone expressing cells, and GABA_{B}R1a alone expressing cells immunoprecipitated with the FLAG-antibody. Since immunoreactive species were detected only in cells co-expressing HG20 and GABA_{B}R1a this experiment demonstrates that HG20 and GABA_{B}R1a can only be co-immunoprecipitated as part of a complex (FIG. 14, lane 4). Based on the predicted molecular mass of a heterodimer of HG20 and GABA_{B}R1a the ˜250+ and ˜130 kDa species may represent a heterodimer and GABA_{B}R1a monomers, respectively. The stability of the HG20/GABA_{B}R1a heterodimer in denaturing and reducing conditions suggests that SDS-stable transmembrane interactions form the heterodimer, as reported previously for β2 adrenergic and dopamine D2 receptors (Ng et al., 1996, Biochem. Biophys. Res. Comm. 227:200-204; Hebert et al., 1996, J. Biol. Chem. 271, 16384-16392). The monomer might result from partial disruption, subsequent to immunoprecipitation, of N-terminal Sushi repeats, C-terminal alpha-helical interacting domains (e.g., coiled-coils) present in HG20 and GABA_{B}R1a subunits, transmembrane interactions, or disulfide bonds that contribute to forming the heterodimer.

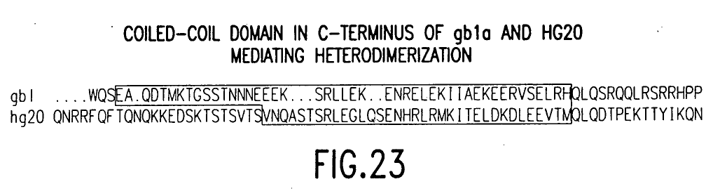

Particular examples of such regions likely to be involved in forming the heterodimer are shown in FIG. 23. Regions such as those shown in FIG. 23, as well as polypeptides comprising such regions are expected to be useful for the purpose of modulating the formation of heterodimers involving HG20 and thus controlling GABA_{B} receptor activity. Accordingly, the present invention includes polypeptides comprising the coiled-coil domains of HG20, GABA_{B}R1a and GABA_{B}R1b. In particular, the present invention includes polypeptides comprising an amino acid sequence selected from the group consisting of: positions 756-829 of SEQ.ID.NO.:2; positions 779-814 of SEQ.ID.NO.:2; positions 886-949 of SEQ.ID.NO.:21; and positions 889-934 of SEQ.ID.NO.:21; where the polypeptides do not contain other contiguous amino acid sequences longer than 5 amino acids from a GABA_{B} receptor subunit. The present invention also includes heterodimers of such polypeptides. In more general terms, the present invention includes comprising a coiled-coil domain from a first GABA_{B} receptor subunit and no other contiguous amino acid sequences longer than 5 amino acids from the first GABA_{B} receptor subunit where the coiled-coil domain is present in the C-terminus of the first GABA_{B} receptor subunit and mediates heterodimerization of the first GABA_{B} receptor subunit with a second GABA_{B} receptor subunit.

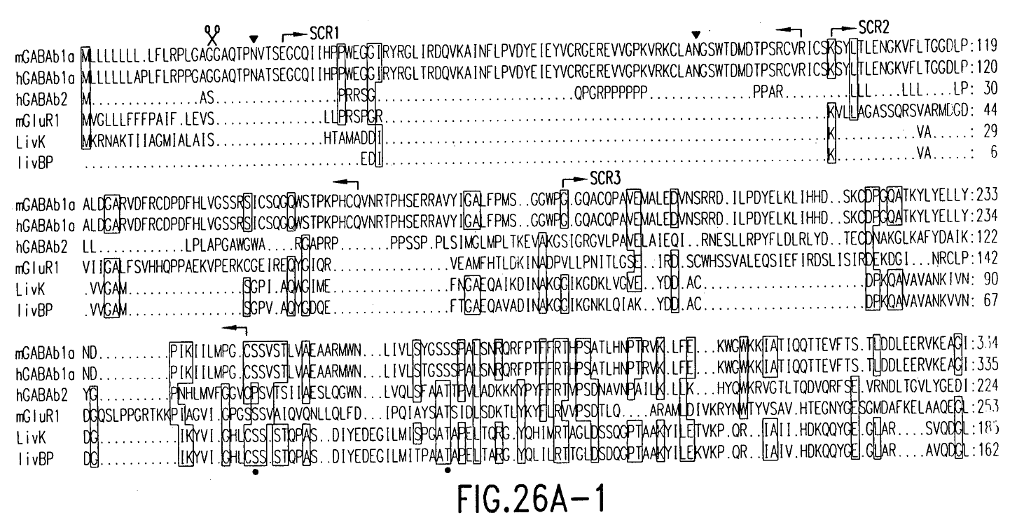

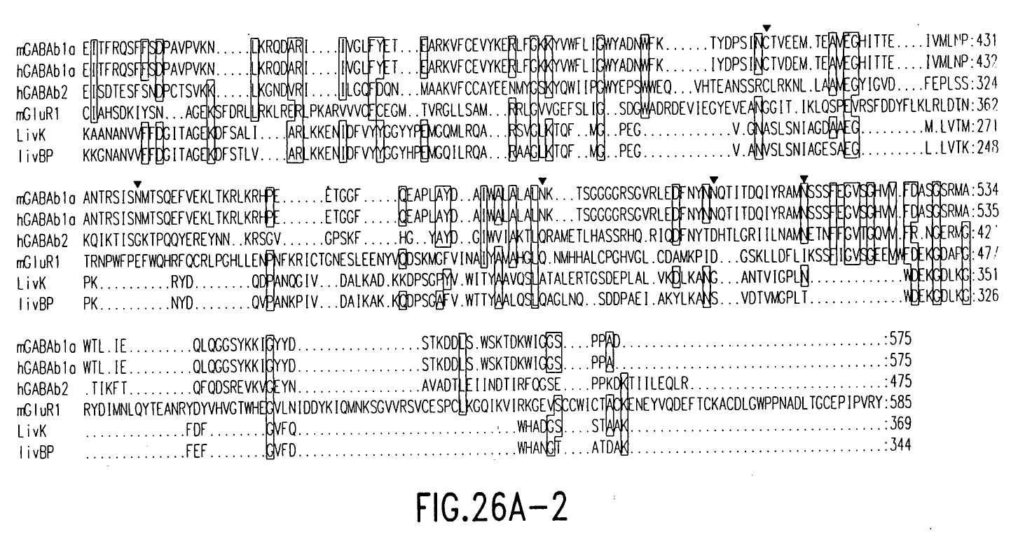

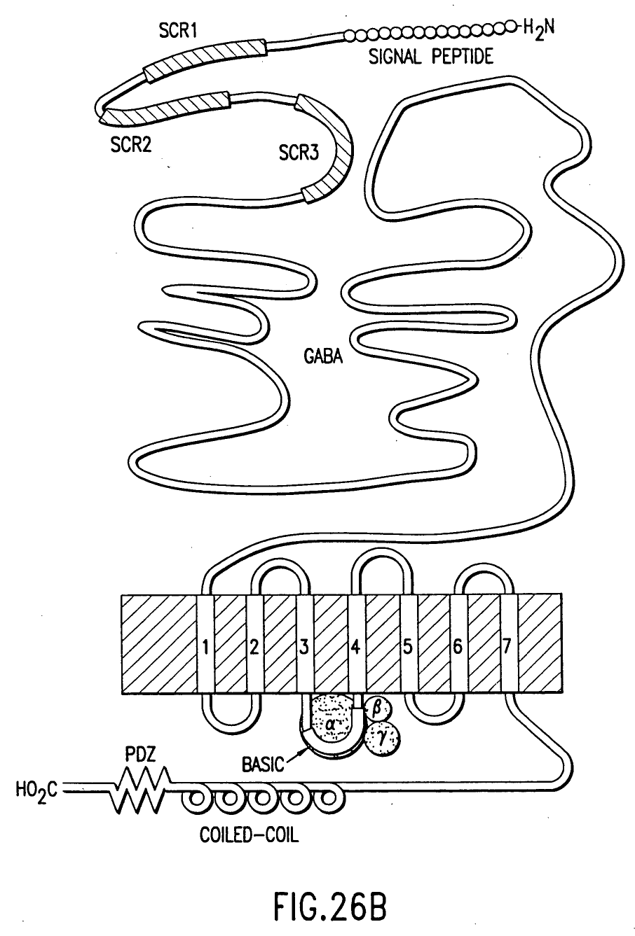

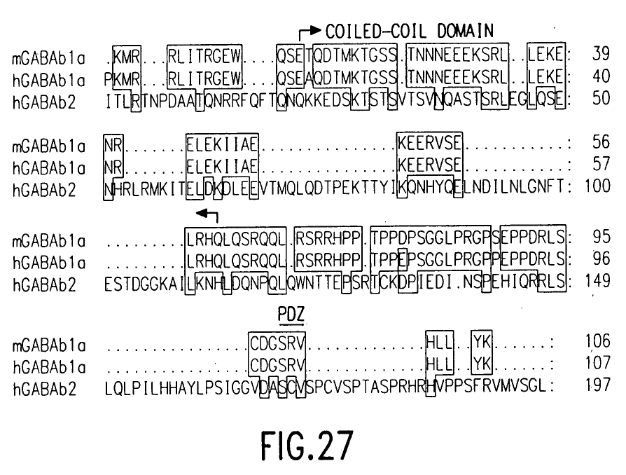

In addition to the coiled-coil domains discussed above, a variety of regions of HG20 and GABA_{B}R1a are expected to be important for heterodimer formation. Motif analysis of the N-terminus of murine GABA_{B}R1a revealed seven consensus N-linked glycosylation sites and three putative short consensus repeats (SCRs) of ˜60 amino acids each: amino acids 27-96 and amino acids 102-157 (GABA_{B}R1a specific), and amino acids 183-245 (common to GABA_{B}R1b (Kaupmann et al., 1997, Nature 386:239-246) and HG20 (Jones et al., 1998, Nature 396:674-679; White et al., 1998, Nature 396:679-682; Kaupmann et al., 1998, Nature 396:683-687; Kuner et al., 1999, Science 283:74-77) not described previously (FIG. 26A-B). Since SCRs are known to play important roles in protein-protein interactions in a wide variety of complement proteins, adhesion proteins, and selections (Chou and Heinrikson, 1997, J. Protein Chem. 16:765-773; Perkins et al., 1998, Biochemistry 27:4004-4012), of which the latter shows weak amino acid identity to murine GABA_{B}R1a these SCRs, together with the coiled-coil domains discussed above in the carboxyl tails of GABA_{B}R1a and HG20 (FIG. 23), are expected to be involved in the heterodimerization of GABA_{B}R1a and HG20.

Therefore, the present invention includes a polypeptide comprising an SCR domain from a first GABA_{B} receptor subunit and no other contiguous amino acid sequences longer than 5 amino acids from the first GABA_{B} receptor subunit where the SCR domain is present in the N-terminus of the first GABA_{B} receptor subunit and mediates heterodimerization of the first GABA_{B} receptor subunit with a second GABA_{B} receptor subunit. In particular embodiments, the SCR is selected from the group consisting of: positions 27-96 of SEQ.ID.NO.:20; positions 102-157 of SEQ.ID.NO.:20; positions 183-245 of SEQ.ID.NO.:20; positions 28-97 of SEQ.ID.NO.:21; positions 103-158 of SEQ.ID.NO.:21; positions 184-246 of SEQ.ID.NO.:21; positions 4-22 of SEQ.ID.NO.:2; positions 23-49 of SEQ.ID.NO.:2; and positions 72-135 of SEQ.ID.NO.:2.

As in the metabotropic glutamate receptors (mGLURs), the second intracellular loop of murine GABA_{B}R1a is rich in basic amino acids which may play a role in G-protein-interactions (reviewed by Pin and Duvoisin, 1995, Neuropharmacology 34:1-26), and, as in the mGLURs, the carboxyl tail of murine GABA_{B}R1a contains a PDZ protein-interacting module (serine-arginine-valine, amino acids 953-955) which has been shown for mGLURs to play an important role in the interactions among the signaling components of synaptic junctions (Brakeman et al. 1997, Nature 386:284-288). The murine GABA_{B}R1a receptor also contains potential protein kinase C and casein kinase II recognition sites predicted using ProSearch (Kolakowski et al., 1992, Biotechniques 13:919-921).

The present invention also relates to the identification and cloning of the murine GABA_{B}R1a receptor, the murine ortholog of the rat GABA_{B}R1a receptor described in Kaupmann et al., 1997, Nature 386:239-246 (Kaupmann). The present invention provides DNA encoding murine GABA_{B}R1a that is substantially free from other nucleic acids. The present invention also provides recombinant DNA molecules encoding murine GABA_{B}R1a.

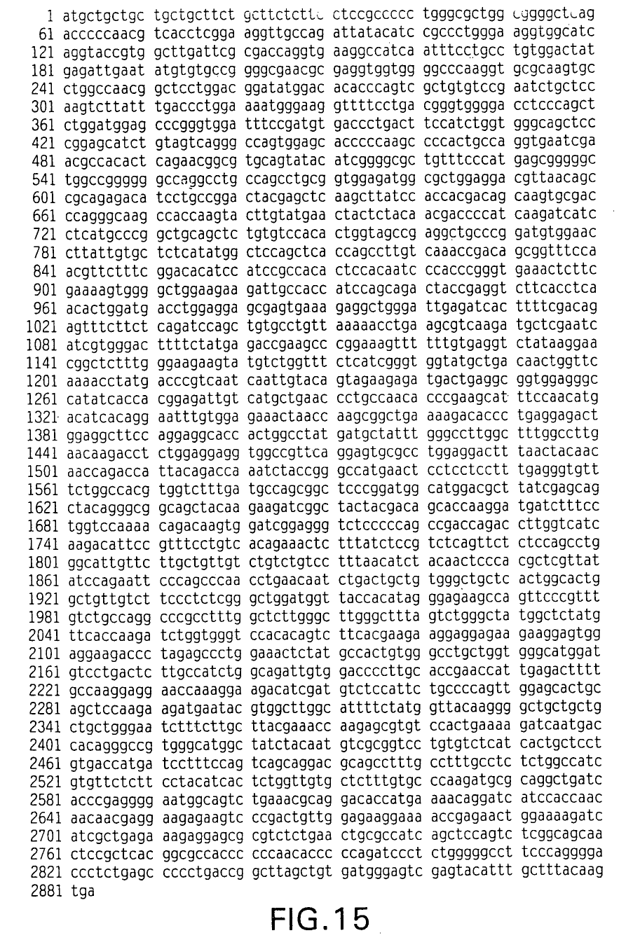

The present invention provides a DNA molecule encoding murine GABA_{B}R1a that is substantially free from other nucleic acids and comprises the nucleotide sequence shown in FIG. 15 as SEQ.ID.NO.:19. The open reading frame of SEQ.ID.NO.:19, encoding mouse GABA_{B}R1a protein, is positions 1-2,880, with positions 2,881-2,883 representing a stop codon. Thus, the present invention also provides a DNA molecule substantially free from other nucleic acids comprising the nucleotide sequence of positions 1-2,880 of SEQ.ID.NO.:19.

Sequence analysis of the open reading frame of the murine GABA_{B}R1a DNA revealed that it encodes a mature protein (i.e., lacking a signal sequence) of 942 amino acids with a predicted molecular weight of 106.5 kDa that is 99% identical to rat GABA_{B}R1a (described in Kaupmann). with only six amino acid changes overall. Murine GABA_{B}R1a protein shares 31% overall amino acid identity to HG20.

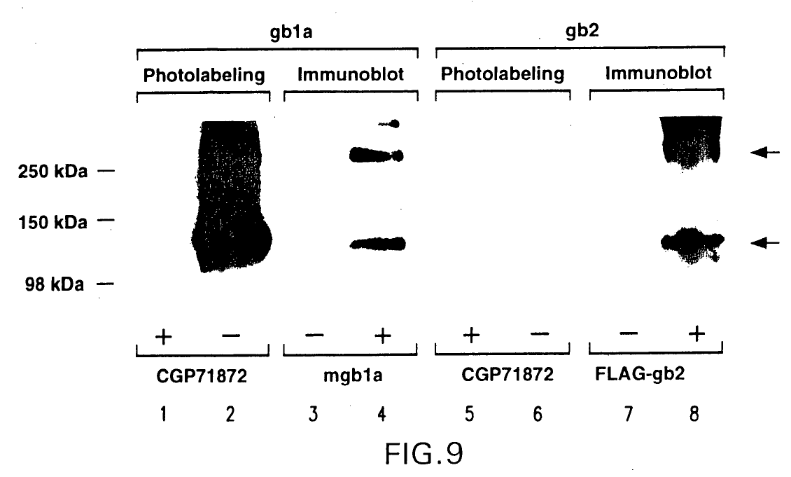

CGP71872 is a photoaffinity ligand specific for GABA_{B}R1a receptors (K_{d}=1.0±0.2 nM) that can be cross-linked to rat GABA_{B}R1a (Kaupmann et al., 1997, Nature 386:239-246). In crude membranes prepared from COS-7 cells transiently transfected with murine GABA_{B}R1a [¹²⁵I]CGP71872 photolabelled a major band at ˜130 kDa representing the mature (presumably glycosylated) protein and an additional band at approximately twice that molecular weight, possibly representing dimers (FIG. 9). Ligand-binding species could also be detected with affinity purified GABA_{B}R1a antibodies 1713.1 (raised against the peptide acetyl-DVNSRRDILPDYELKLC-amide; a port ion of SEQ.ID.NO.:20) and 1713.2 (raised against the peptide acetyl-CATLHNPTRVKLFEK-amide; a portion of SEQ.ID.NO.:20) (FIG. 9). In contrast, FLAG-tagged HG20 protein did not bind the high-affinity CGP71872 ligand, although expression of the protein was confirmed by immunoblot analysis (FIG. 9).

Displacement of [¹²⁵I]CGP71872 binding to recombinant murine GABA_{B}R1a was in the appropriate rank order of potency for GABAergic ligands: CGP71872>SKF-97541 (3-aminopropyl(methyl)-phosphinic acid)>GABA>(−)baclofen>saclofen>(L)-glutamic acid. Interestingly, recombinant rat GABA_{B}R1a exhibits 10-25 fold lower affinity for agonists than native GABA_{B} receptors in brain (Kaupmann et al., 1997, Nature 386:239). Although the reason for this discrepancy remains unclear, a recent report indicated that recombinant GABA_{B}R1a may require additional cellular components for functional targeting to the plasma membrane (Couve et al., 1998, J. Biol. Chem. 273:26361-26367). Thus, GABA_{B}R1a alone, without such additional components, might be expected to exhibit somewhat altered ligand binding characteristics.

In the binding experiments discussed above using GABA_{B}R1a alone, surprisingly, dose-dependent displacement was not detected for (+)baclofen, and the affinities of agonists (GABA, SKF-97541, and (−)baclofen) and partial agonists ((+)baclofen, saclofen, (L)-glutamic acid) but not the affinity of antagonist (CGP71872) for the recombinant GABA_{B}R1a were markedly lower compared to native receptors in rat brain (Table 1). Agonist affinities of co-expressed HG20 and GABA_{B}R1a were examined in membranes prepared from cells co-expressing GABA_{B}R1a and FLAG-tagged HG20. Competition of [¹²⁵I]CGP71872 binding in these membranes showed recovery of high-affinity ligand binding comparable to native receptors in rat brain (Table 1). The simplest explanation for these results is that the high-affinity agonist binding pocket may comprise interactions between the N-terminal domains of HG20 and GABA_{B}R1a that form the heterodimer.

TABLE 1

| Ligand | rat cortex* | | gb1a | | gb1a/gb2 | |
|---|---|---|---|---|---|---|
| CGP71872 | 0.5 | nM | 0.52-0.67 | nM | 0.15-0.27 | nM |
| GABA | 2.5 | uM | 42.55-68.38 | uM | 1.77-2.55 | uM |
| SKF-97541** | not | | 11.09-11.47 | uM | 0.80-0.96 | uM |
| | determined | | | | | |
| (−)Baclofen | 0.5 | uM | 31.46-53.70 | uM | 3.92-7.78 | uM |
| (+)Baclofen | not | | no fit | | 1.25-3.94 | mM |
| | determined | | | | | |
| Saclofen | 156 | uM | 280.5-365.0 | uM | 119.4-131.4 | uM |

| | | | | | |
|---|---|---|---|---|---|
| L-Glutamate | not | 119.4-285.0 | mM | 116.2-201.6 | mM |
| | determined | | | | |

*reported by Kaupmann et al., (1997) Nature 386, 239-246
**3-aminopropyl(methyl)phosphinic acid

In Table 1, gb1a refers to GABA_{B}R1a and gb1a/gb2 refers to HG20/GABA_{B}R1a heterodimers.

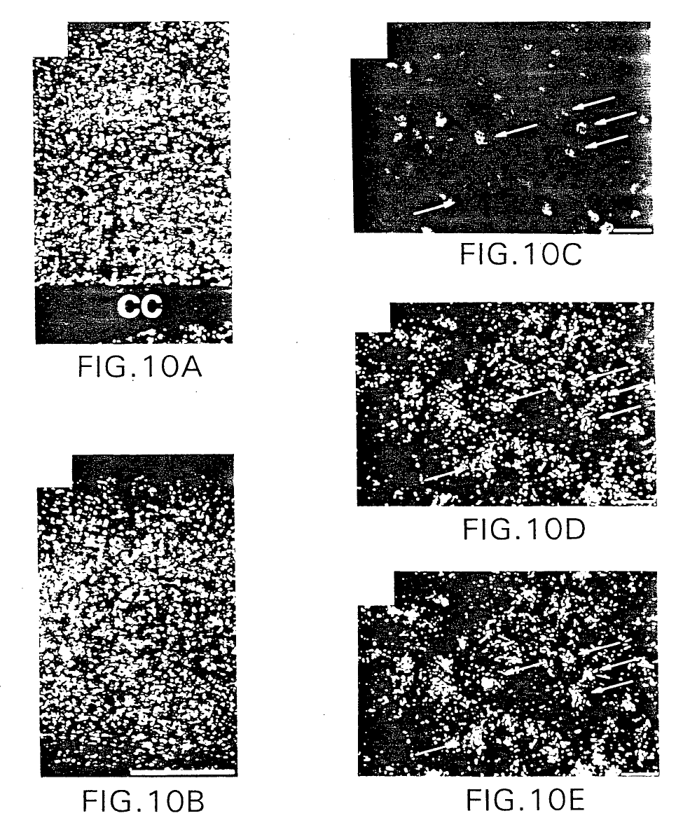

Co-localization studies were performed to determine if mRNAs for GABA_{B}R1a and HG20 co-exist in the same cells in the brain. FIG. 10A-B shows equivalent levels of GABA_{B}R1a and HG20 hybridization in adjacent coronal sections of rat parietal cortex, indicating that messages for both receptors are expressed in this brain region. Radiolabelled and fluorescent probes for the two receptors were used to look at the cellular level where it was observed that message for both receptors is expressed in the same cells (Example 13 and FIG. 10C-E). In the parietal cortex and all other major brain regions studied, including the hippocampus, thalamus, cerebellum, and vestibular ganglion, GABA_{B}R1a and HG20 mRNAs are co-localized in the same cells. These results suggest that the functional native GABA_{B} receptors found in these cells involve both GABA_{B}R1a and HG20. Co-immunoprecipitation, functional, and anatomical data described herein converge to strongly support the argument that the native, functional GABA_{B} receptor is a heterodimer of GABA_{B}R1a and HG20. This work is particularly exciting because it represents the first example of a heteromeric G protein-coupled receptor.

The novel murine GABA_{B}R1a DNA sequences of the present, in whole or in part, can be linked with other DNA sequences, i.e., DNA sequences to which GABA_{B}R1a DNA is not naturally linked, to form “recombinant DNA molecules” encoding murine GABA_{B}R1a. Such other sequences can include DNA sequences that control transcription or translation such as, e.g., translation initiation sequences, promoters for RNA polymerase II, transcription or translation termination sequences, enhancer sequences, sequences that control replication in microorganisms, or that confer antibiotic resistance. The novel DNA sequences of the present invention can be inserted into vectors such as plasmids, cosmids, viral vectors, or yeast artificial chromosomes.

The present invention also includes isolated forms of DNA encoding GABA_{B}R1a. By “isolated DNA encoding GABA_{B}R1a” is meant DNA encoding GABA_{B}R1a that has been isolated from a natural source or produced by recombinant means. Use of the term “isolated” indicates that DNA encoding GABA_{B}R1a is not present in its normal cellular environment. Thus, an isolated DNA encoding GABA_{B}R1a may be in a cell-free solution or placed in a different cellular environment from that in which it occurs naturally. The term isolated does not imply that isolated DNA encoding GABA_{B}R1a is the only DNA present. but instead means that isolated DNA encoding GABA_{B}R1a is at least 95% free of non-nucleic acid material (e.g., proteins, lipids, carbohydrates) naturally associated with the DNA encoding GABA_{B}R1a. Thus, DNA encoding GABA_{B}R1a that is expressed in bacteria or even in eukaryotic cells which do not naturally (i.e., without human intervention) contain it through recombinant means is “isolated DNA encoding GABA_{B}R1a.”

Another aspect of the present invention includes host cells that have been engineered to contain and/or express DNA sequences encoding murine GABA_{B}R1a. Such recombinant host cells can be cultured under suitable conditions to produce murine GABA_{B}R1a protein. An expression vector containing DNA encoding the murine GABA_{B}R1a protein can be used for expression of the murine GABA_{B}R1a protein in a recombinant host cell. Recombinant host cells may be prokaryotic or eukaryotic, including but not limited to, bacteria such as E. coli, fungal cells such as yeast, mammalian cells including, but not limited to, cell lines of human, bovine, porcine, monkey and rodent origin, and insect cells including but not limited to Drosophila and silkworm derived cell lines. Cell lines derived from mammalian species which are suitable for recombinant expression of the murine GABA_{B}R1a protein and which are commercially available, include but are not limited to, L cells L-M(TK⁻) (ATCC CCL 1.3), L cells L-M (ATCC CCL 1.2), HEK293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171), Xenopus melanophores, and Xenopus oocytes.

A variety of mammalian expression vectors can be used to express recombinant murine GABA_{B}R1a in mammalian cells. Commercially available mammalian expression vectors which are suitable include, but are not limited to, pMC1neo (Stratagene), pSG5 (Stratagene), pcDNAI and pcDNAIamp, pcDNA3, pcDNA3.1, pCR3.1 (Invitrogen), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), and the PT7TS oocyte expression vector (or similar expression vectors containing the globin 5′ UTR and the globin 3′ UTR). Following expression in recombinant cells, the murine GABA_{B}R1a protein can be purified by conventional techniques to a level that is substantially free from other proteins.

Other cells that are particularly suitable for expression of the murine GABA_{B}R1a protein are immortalized melanophore pigment cells from Xenopus laevis. Such melanophore pigment cells can be used for functional assays using recombinant expression of murine GABA_{B}R1a in a manner similar to the use of such melanophore pigment cells for the functional assay of other recombinant GPCRs (Graminski et al., 1993, J. Biol. Chem. 268:5957-5964; Lerner, 1994, Trends Neurosci. 17:142-146; Potenza & Lerner, 1992, Pigment Cell Res. 5:372-378; Potenza et al., 1992, Anal. Biochem. 206:315-322).

The present invention includes a method of producing the murine GABA_{B}R1a protein comprising:
(a) transfecting a host cell with a expression vector comprising DNA that encodes the murine GABA_{B}R1a protein;
(b) growing the host cells under conditions such that the murine GABA_{B}R1a protein is produced; and
(c) recovering the murine GABA_{B}R1a protein from the host cells.

In particular embodiments, the method of recovering the murine GABA_{B}R1a protein may involve obtaining membrane preparations from the host cells that contain the murine GABA_{B}R1a protein. Such membrane preparations may contain heterodimers of GABA_{B}R1a protein and HG20 protein that form functional GABA_{B} receptors.

In particular embodiments, the cells are eukaryotic cells. In other embodiments, the cells are mammalian cells. In still other embodiments, the cells are COS cells, in particular COS-7 cells (ATCC CRL 1651), COS-1 cells (ATCC CRL 1650), HEK293 cells (ATCC CRL 1573), or Xenopus melanophores.

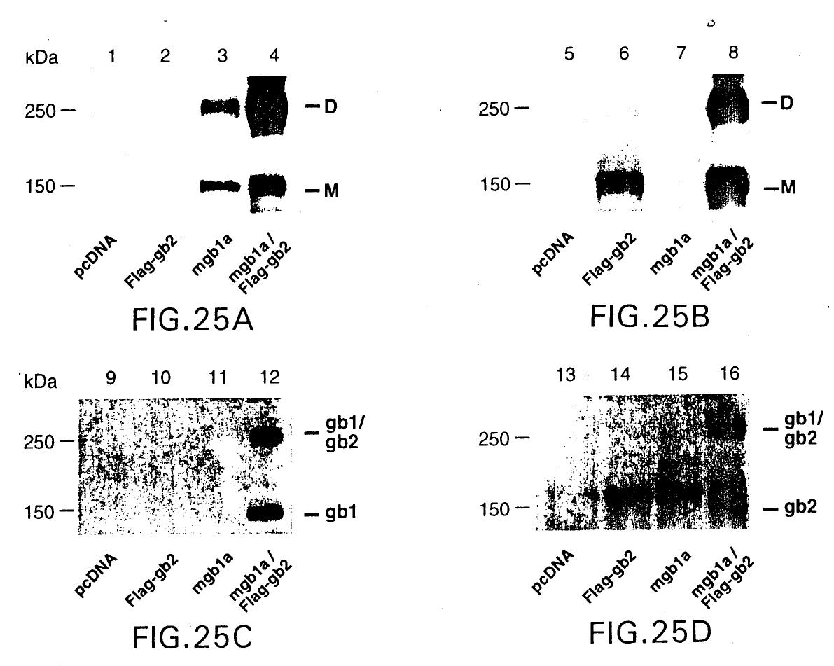

The present inventors have discovered that, when either HG20 or GABA_{B}R1a subunits are recombinantly expressed separately, i.e., in different cells, very little or no expression is observed. It is only when HG20 and GABA_{B}R1a subunits are recombinantly co-expressed, i.e., expressed in the same cells at the same time, that high level expression of HG20 and GABA_{B}R1a is observed (see FIG. 25). Given the close relationship among GABA_{B}R1a GABA_{B}R1b, C. elegans genes related to GABA_{B}R1a and HG20 (see FIG. 24), and the close relationship that is expected to be found between other isoforms of GABA_{B}R1a and GABA_{B}R1b, it is believed that co-expression of HG20 and either GABA_{B}R1a GABA_{B}R1b, C. elegans genes related to GABA_{B}R1a and HG20, or other isoforms of GABA_{B}R1a and GABA_{B}R1b will also result in increased expression of HG20 and GABA_{B}R1a GABA_{B}R1b, C. elegans genes related to GABA_{B}R1a and HG20, or other isoforms of GABA_{B}R1a and GABA_{B}R1b as compared to expression of these proteins separately.

Accordingly, the present invention includes a method of co-expressing HG20 and GABA_{B}R1a, GABA_{B}R1b, C. elegans genes related to GABA_{B}R1a and HG20, or other isoforms of GABA_{B}R1a and GABA_{B}R1b so as to result in an increase in expression of HG20 and GABA_{B}R1a GABA_{B}R1b, C. elegans genes related to GABA_{B}R1a and HG20, or other isoforms of GABA_{B}R1a and GABA_{B}R1b as compared to expression when HG20 and GABA_{B}R1a GABA_{B}R1b, C. elegans genes related to GABA_{B}R1a and HG20, or other isoforms of GABA_{B}R1a and GABA_{B}R1b are expressed separately. In particular embodiments, the level of expression of HG20, GABA_{B}R1a GABA_{B}R1b, C. elegans genes related to GABA_{B}R1a and HG20, or other isoforms of GABA_{B}R1a and GABA_{B}R1b is measured in the co-expressing cells. In particular embodiments, the level of expression of HG20, GABA_{B}R1a GABA_{B}R1b, C. elegans genes related to GABA_{B}R1a and HG20, or other isoforms of GABA_{B}R1a and GABA_{B}R1b is measured by immunoblot or by immunoprecipitation/immunoblotting methods.

Thus, the present invention includes a method of increasing expression of HG20 and GABA_{B}R1a GABA_{B}R1b, C. elegans genes related to GABA_{B}R1a and HG20, or other isoforms of GABA_{B}R1a and GABA_{B}R1b comprising:
(a) recombinantly expressing HG20 and GABA_{B}R1a GABA_{B}R1b, C. elegans genes related to GABA_{B}R1a and HG20, or other isoforms of GABA_{B}R1a and GABA_{B}R1b in the same cells;
(b) measuring the expression of HG20, GABA_{B}R1a GABA_{B}R1b, C. elegans genes related to GABA_{B}R1a and HG20, or other isoforms of GABA_{B}R1a and GABA_{B}R1b, where a measurement of detectable expression of HG20, GABA_{B}R1a GABA_{B}R1b, C. elegans genes related to GABA_{B}R1a and HG20, or other isoforms of GABA_{B}R1a and GABA_{B}R1b indicates that increased expression has been achieved.

In particular embodiments, the measurement of expression is carried out by immunoblotting with or without immunoprecipitation.

In other embodiments, the method also comprises the steps of recombinantly expressing HG20 and GABA_{B}R1a GABA_{B}R1b, C. elegans genes related to GABA_{B}R1a and HG20, or other isoforms of GABA_{B}R1a and GABA_{B}R1b separately, measuring the level of expression of HG20, GABA_{B}R1a GABA_{B}R1b, C. elegans genes related to GABA_{B}R1a and HG20, or other isoforms of GABA_{B}R1a and GABA_{B}R1b in the separately expressing cells, and comparing the amount of expression of HG20, GABA_{B}R1a GABA_{B}R 1b, C. elegans genes related to GABA_{B}R1a and HG20, or other isoforms of GABA_{B}R1a and GABA_{B}R1b in the separately expressing cells to the amount of expression of HG20, GABA_{B}R1a GABA_{B}R1b, C. elegans genes related to GABA_{B}R1a and HG20, or other isoforms of GABA_{B}R1a and GABA_{B}R1b in the co-expressing cells.

Accordingly, the present invention includes a method of increasing expression of HG20 and GABA_{B}R1a, GABA_{B}R1b, C. elegans genes related to GABA_{B}R1a and HG20, or other isoforms of GABA_{B}R1a and GABA_{B}R1b comprising:
(a) recombinantly expressing HG20 and GABA_{B}R1a GABA_{B}R1b, C. elegans genes related to GABA_{B}R1a and HG20, or other isoforms of GABA_{B}R1a and GABA_{B}R1b in the same cells to form co-expressing cells;
(b) recombinantly expressing HG20 and GABA_{B}R1a GABA_{B}R1b, C. elegans genes related to GABA_{B}R1a and HG20, or other isoforms of GABA_{B}R1a and GABA_{B}R1b in different cells to form separately expressing cells;
(c) measuring the expression of HG20, GABA_{B}R1a GABA_{B}R1b, C. elegans genes related to GABA_{B}R1a and HG20, or other isoforms of GABA_{B}R1a and GABA_{B}R1b in the co-expressing cells;
(d) measuring the expression of HG20, GABA_{B}R1a GABA_{B}R1b, C. elegans genes related to GABA_{B}R1a and HG20, or other isoforms of GABA_{B}R1a and GABA_{B}R1b in the separately expressing cells;
where if the amount of expression of HG20, GABA_{B}R1a GABA_{B}R1b, C. elegans genes related to GABA_{B}R1a and HG20, or other isoforms of GABA_{B}R1a and GABA_{B}R1b is greater in the co-expressing cells as compared to the separately expressing cells, this indicates that increased expression has been achieved.

In particular embodiments, the measurement of expression is carried out by immunoblotting with or without immunoprecipitation.

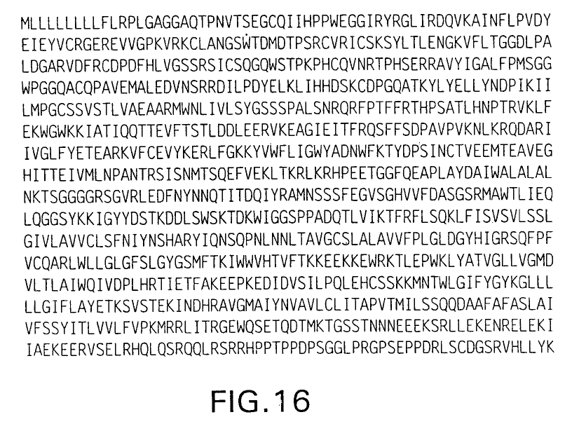

The present invention includes murine GABA_{B}R1a protein substantially free from other proteins. The amino acid sequence of the full-length murine GABA_{B}R1a protein is shown in FIG. 16 as SEQ.ID.NO.:20. Thus, the present invention includes polypeptides comprising the murine GABA_{B}R1a protein substantially free from other proteins having the amino acid sequence SEQ.ID.NO.:20. The present invention also includes murine GABA_{B}R1a protein lacking a signal sequence as well as DNA encoding such a protein. Such a murine GABA_{B}R1a protein lacking a signal sequence is represented by amino acids 18-960 of SEQ.ID.NO.:20.

The present invention includes modified murine GABA_{B}R1a polypeptides which have amino acid deletions, additions, or substitutions but that still retain substantially the same biological activity as native murine GABA_{B}R1a protein. The present invention includes polypeptides where one amino acid substitution has been made in SEQ.ID.NO.:20 or in a polypeptide represented by SEQ.ID.NO.:20 lacking a signal sequence, wherein the polypeptides still retain substantially the same biological activity as native murine GABA_{B}R1a protein. The present invention also includes polypeptides where two or more amino acid substitutions have been made in SEQ.ID.NO.:20 or in a polypeptide represented by SEQ.ID.NO.:20 lacking a signal sequence, wherein the polypeptides still retain substantially the same biological activity as native murine GABA_{B}R1a protein. In particular, the present invention includes embodiments where the above-described substitutions are conservative substitutions. In particular, the present invention includes embodiments where the above-described substitutions do not occur in the ligand-binding domain of native murine GABA_{B}R1a protein. In particular, the present invention includes embodiments where amino acid changes have been made in positions of native murine GABA_{B}R1a protein where the amino acid sequence of native murine GABA_{B}R1a protein differs from the amino acid sequence of HG20 when the amino acid sequences of native murine GABA_{B}R1a protein and HG20 are aligned in a manner similar to the alignment of the amino acid sequences of GABA_{B}R1b protein and HG20 shown in FIG. 8.

The present invention also includes isolated forms of murine GABA_{B}R1a proteins. By “isolated murine GABA_{B}R1a protein” is meant murine GABA_{B}R1a protein that has been isolated from a natural source or produced by recombinant means. Use of the term “isolated” indicates that murine GABA_{B}R1a protein is not present in its normal cellular environment. Thus, an isolated murine GABA_{B}R1a protein may be in a cell-free solution or placed in a different cellular environment from that in which it occurs naturally. The term isolated does not imply that an isolated murine GABA_{B}R1a protein is the only protein present. but instead means that an isolated murine GABA_{B}R1a protein is at least 95% free of non-amino acid material (e.g., nucleic acids, lipids, carbohydrates) naturally associated with the murine GABA_{B}R1a protein. Thus, an murine GABA_{B}R1a protein that is expressed in bacteria or even in eukaryotic cells which do not naturally (i.e., without human intervention) express it through recombinant means is an “isolated murine GABA_{B}R1a protein.”

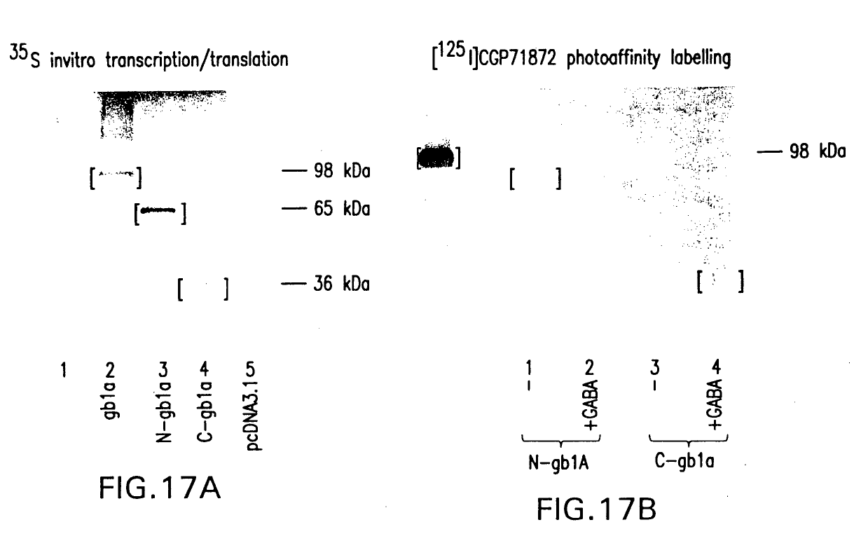

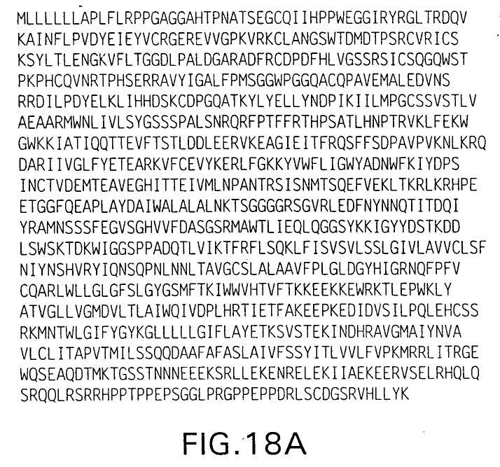

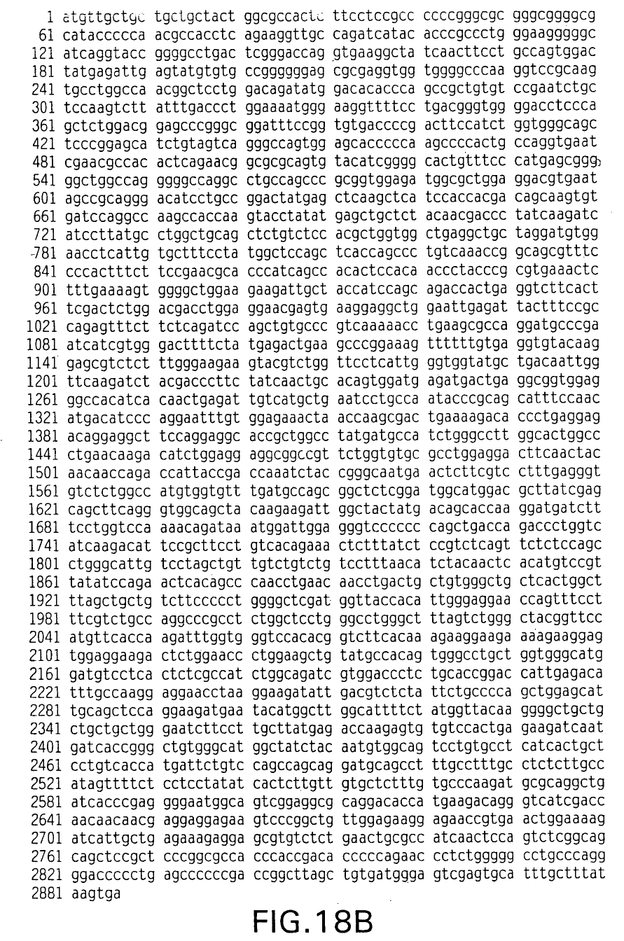

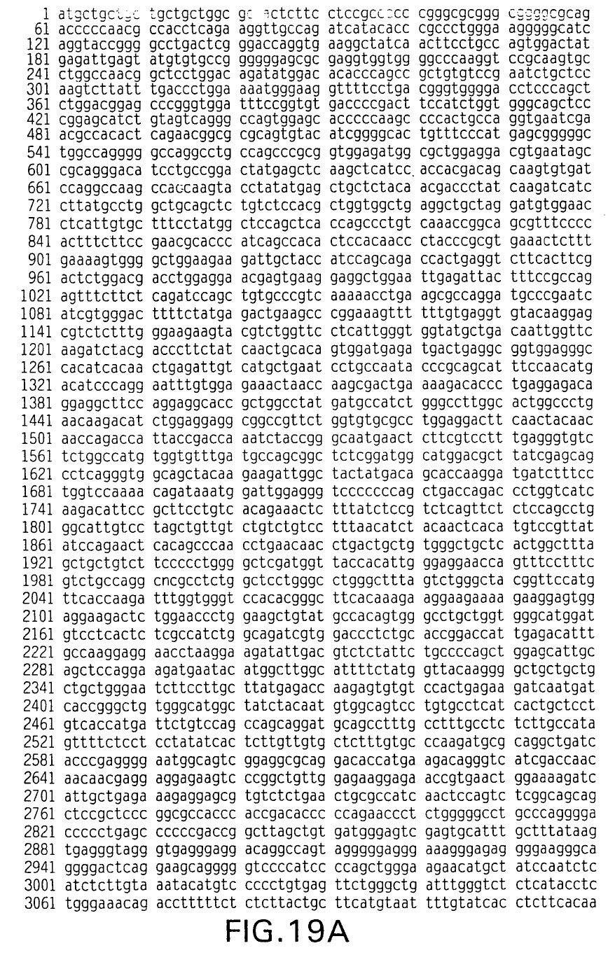

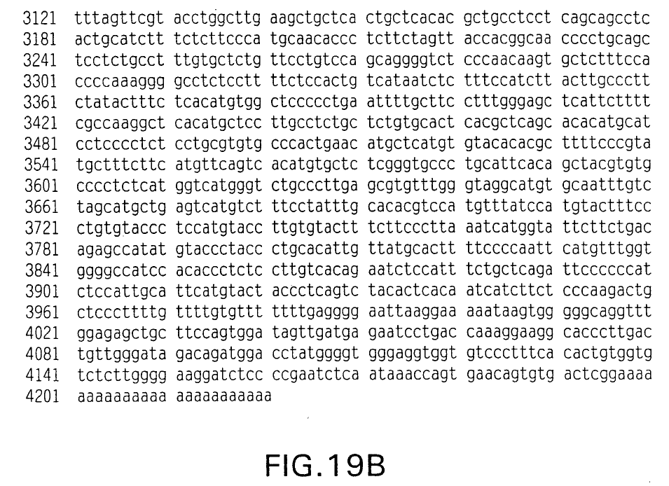

The present invention also provides ligand-binding domains of murine GABA_{B}R1a protein. A FASTA search of the database GenBank (bacterial division) using the N-terminal domain of murine GABA_{B}R1a (amino acid positions 147-551 of SEQ.ID.NO.:20) as the probe reveals a match with the E. coli leucine-specific binding protein (livK) (22% identity over 339 amino acids), whereas no match to any bacterial amino acid binding protein is found using the receptor sequence inclusive of the region that includes the seven transmembrane domains (TM 1-7; amino acid positions 552-960) as a probe. The ligand-binding domain(s) of GABA_{B}R1a was also experimentally determined. Photoaffinity [¹²⁵I]CGP71872 labeling experiments provided direct physical evidence that the N-terminal extracellular domain but not a C-terminal fragment of GABA_{B}R1a (comprising TM1-7 and inclusive to the carboxyl tail) is responsible for ligand-binding (see Examples 14-19 and FIG. 17B).

Accordingly, the present invention includes a polypeptide comprising the ligand binding domain of murine GABA_{B}R1a. In preferred embodiments, the polypeptide comprises amino acids 147-551 of SEQ.ID.NO.:20.

The present invention includes methods of identifying compounds that specifically bind to the GABA_{B} receptor, as well as compounds identified by such methods. The specificity of binding of compounds showing affinity for the GABA_{B} receptor is shown by measuring the affinity of the compounds for recombinant cells expressing HG20 and either GABA_{B}R1a or GABA_{B}R1b, or for membranes from such cells. Expression of the GABA_{B} receptor and screening for compounds that bind to the GABA_{B} receptor or that inhibit the binding of a known, radiolabelled ligand of the GABA_{B} receptor, e.g., an amino acid or a GABA analogue such as (−)baclofen, to these cells, or membranes prepared from these cells, provides an effective method for the rapid selection of compounds with high affinity for the GABA_{B} receptor. Other radiolabelled ligands that might be used are ibotenic acid, the amino acids glutamate and glycine, other amino acids, decarboxylated amino acids, or any of the other GABA_{B} receptor ligands disclosed herein or known in the art. Such ligands need not necessarily be radiolabelled but can also be nonisotopic compounds that can be used to displace bound radiolabelled compounds or that can be used as activators in functional assays. Compounds identified by the methods disclosed herein are likely to be agonists or antagonists of the GABA_{B} receptor and may be peptides, proteins, or non-proteinaceous organic molecules.

Therefore, the present invention includes assays by which GABA_{B} receptor agonists and antagonists can be identified. Methods for identifying agonists and antagonists of other receptors are well known in the art and can often be adapted to identify agonists and antagonists of the GABA_{B} receptor. Accordingly, the present invention includes a method for determining whether a substance binds GABA_{B} receptors and is thus a potential agonist or antagonist of the GABA_{B} receptor that comprises:
(a) providing cells comprising an expression vector encoding HG20 and an expression vector encoding GABA_{B}R1a or GABA_{B}R1b;
(b) culturing the cells under conditions such that HG20 and GABA_{B}R1a or GABA_{B}R1b are expressed and heterodimers of HG20 and GABA_{B}R1a or GABA_{B}R1b are formed;
(c) exposing the cells to a labeled ligand of GABA_{B} receptors in the presence and in the absence of the substance;
(d) measuring the binding of the labeled ligand to the heterodimers of HG20 and GABA_{B}R1a or GABA_{B}R1b in the presence and in the absence of the substance;
where if the amount of binding of the labeled ligand is less in the presence of the substance than in the absence of the substance, then the substance is a potential agonist or antagonist of GABA_{B} receptors.

Examples of ligands of GABA_{B} receptors are: CGP71872, GABA, saclofen, (−)baclofen, glycine, and (L)-glutamic acid.

The present invention also includes a method for determining whether a substance is capable of binding to GABA_{B} receptors, i.e., whether the substance is a potential agonist or an antagonist of GABA_{B} receptors, where the method comprises:
(a) providing test cells comprising an expression vector encoding HG20 and an expression vector encoding GABA_{B}R1a or GABA_{B}R1b;
(b) culturing the test cells under conditions such that HG20 and GABA_{B}R1a or GABA_{B}R1b are expressed and heterodimers of HG20 and GABA_{B}R1a or GABA_{B}R1b are formed;
(c) exposing the test cells to the substance;
(d) measuring the amount of binding of the substance to the test cells;
(e) measuring the amount of binding of the substance to control cells;
(f) comparing the amount of binding of the substance to the test cells with the amount of binding of the substance to control cells;
where if the amount of binding of the substance to the test cells is greater than the amount of binding of the substance to control cells, then the substance is capable of binding to GABA_{B} receptors;
where the control cells are essentially the same as the test cells except that the control cells do not comprise an expression vector encoding HG20 and an expression vector encoding GABA_{B}R1a or GABA_{B}R1b.

Once a substance has been identified by the above-described methods, determining whether the substance is an agonist or antagonist can then be accomplished by the use of functional assays such as those described herein.

In particular embodiments, the cells are transfected with an expression vector encoding HG20 and an expression vector encoding GABA_{B}R1a or GABA_{B}R1b.

In particular embodiments, the binding affinity of the substance for the test cells is determined. In particular embodiments, such binding affinity is between 1 nM and 200 mM; preferably between 5 nM and 1 mM; more preferably between 10 nM and 100 μM; and even more preferably between 10 nM and 100 nM.

The conditions under which step (c) of the above-described methods is practiced are conditions that are typically used in the art for the study of protein-ligand interactions: e.g., physiological pH; salt conditions such as those represented by such commonly used buffers as PBS or in tissue culture media; a temperature of about 4° C. to about 55° C.

In a particular embodiment of the above-described methods, the cells are eukaryotic cells. In another embodiment, the cells are mammalian cells. In other embodiments, the cells are L cells L-M(TK⁻) (ATCC CCL 1.3), L cells L-M (ATCC CCL 1.2), HEK293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171), or Xenopus melanophores.

The assays described above can be carried out with cells that have been transiently or stably transfected with an expression vector encoding HG20 and an expression vector encoding GABA_{B}R1a or GABA_{B}R1b. Transfection is meant to include any method known in the art for introducing HG20 and GABA_{B}R1a or GABA_{B}R1b into the test cells. For example, transfection includes calcium phosphate or calcium chloride mediated transfection, lipofection, infection with a retroviral construct, and electroporation. In particular embodiments, a single expression vector encodes HG20 and GABA_{B}R1a or GABA_{B}R1b.

Where binding of the substance or ligand is measured, such binding can be measured by employing a labeled substance or ligand. The substance or ligand can be labeled in any convenient manner known to the art, e.g., radioactively, fluorescently, enzymatically.

In particular embodiments of the above-described methods, the substance or ligand is an amino acid or an amino acid analogue such as CGP71872, GABA, saclofen, (−)baclofen, glycine, and (L)-glutamic acid.

In particular embodiments of the above-described methods, HG20 has an amino acid sequence of SEQ.ID.NO.:2.

In particular embodiments of the above-described methods, HG20 comprises an amino acid sequence selected from the group consisting of:
SEQ.ID.NO.:2;
Positions 9-941 of SEQ.ID.NO.:2;
Positions 35-941 of SEQ.ID.NO.:2;
Positions 36-941 of SEQ.ID.NO.:2;
Positions 38-941 of SEQ.ID.NO.:2;
Positions 39-941 of SEQ.ID.NO.:2;
Positions 42-941 of SEQ.ID.NO.:2;
Positions 44-941 of SEQ.ID.NO.:2;
Positions 46-941 of SEQ.ID.NO.:2;
Positions 52-941 of SEQ.ID.NO.:2; and
Positions 57-941 of SEQ.ID.NO.:2.

In particular embodiments, GABA_{B}R1a is murine GABA_{B}R1a and has the amino acid sequence SEQ.ID.NO.:20. In particular embodiments, GABA_{B}R1a is rat GABA_{B}R1a and has the amino acid sequence reported in Kaupmann et al., 1997, Nature 386:239-246. In particular embodiments, GABA_{B}R1b is rat GABA_{B}R1b and has the amino acid sequence reported in Kaupmann et al., 1997, Nature 386:239-246. In particular embodiments, GABA_{B}R1a is human GABA_{B}R1a and has an amino acid sequence selected from the group consisting of: SEQ.ID.NO.:21 and the protein encoded by SEQ.ID.NO.:23.

The above-described methods can be modified in that, rather than exposing cells to the substance, membranes can be prepared from the cells and those membranes can be exposed to the substance. Such a modification utilizing membranes rather than cells is well known in the art with respect to other receptors and is described in, e.g., Hess et al., 1992, Biochem. Biophys. Res. Comm. 184:260-268.

As a further modification of the above-described method, RNA encoding HG20 and GABA_{B}R1a or GABA_{B}R1b can be prepared as, e.g., by in vitro transcription using a plasmid containing HG20 and a plasmid containing GABA_{B}R1a or GABA_{B}R1b under the control of a bacteriophage T7 promoter, and the RNA can be microinjected into Xenopus oocytes in order to cause the expression of HG20 and GABA_{B}R1a or GABA_{B}R1b in the oocytes. Substances are then tested for binding to the heterodimer of HG20 and GABA_{B}R1a or GABA_{B}R1b expressed in the oocytes. Alternatively, rather than detecting binding, the effect of the substances on the electrophysiological properties of the oocytes can be determined.

The present invention includes assays by which GABA_{B} receptor agonists and antagonists may be identified by their ability to stimulate or antagonize a functional response mediated by the GABA_{B} receptor in cells that have been co-transfected with and that co-express HG20 and GABA_{B}R1a or GABA_{B}R1b.

Accordingly, the present invention provides a method of identifying agonists and antagonists of HG20 comprising:
(a) providing test cells by transfecting cells with:
   (1) an expression vector that directs the expression of HG20 in the cells; and
   (2) an expression vector that directs the expression of GABA_{B}R1a or GABA_{B}R1b in the cells;
(b) exposing the test cells to a substance that is suspected of being an agonist of the GABA_{B} receptor;
(c) measuring the amount of a functional response of the test cells that have been exposed to the substance;
(d) comparing the amount of the functional response exhibited by the test cells with the amount of the functional response exhibited by control cells;
wherein if the amount of the functional response exhibited by the test cells differs from the amount of the functional response exhibited by the control cells, the substance is an agonist or antagonist of the GABA_{B} receptor;
where the control cells are cells that have not been transfected with HG20 and GABA_{B}R1a or GABA_{B}R1b but have been exposed to the substance or are test cells that have not been exposed to the substance.

In particular embodiments of the above-described methods, HG20 has an amino acid sequence of SEQ.ID.NO.:2.

In particular embodiments of the above-described methods, HG20 comprises an amino acid sequence selected from the group consisting of:
SEQ.ID.NO.:2;
Positions 9-941 of SEQ.ID.NO.:2;
Positions 35-941 of SEQ.ID.NO.:2;
Positions 36-941 of SEQ.ID.NO.:2;
Positions 38-941 of SEQ.ID.NO.:2;
Positions 39-941 of SEQ.ID.NO.:2;
Positions 42-941 of SEQ.ID.NO.:2;
Positions 44-941 of SEQ.ID.NO.:2;
Positions 46-941 of SEQ.ID.NO.:2;
Positions 52-941 of SEQ.ID.NO.:2; and
Positions 57-941 of SEQ.ID.NO.:2.

In particular embodiments, GABA_{B}R1a is murine GABA_{B}R1a and has the amino acid sequence SEQ.ID.NO.:20. In particular embodiments, GABA_{B}R1a is rat GABA_{B}R1a and has the amino acid sequence reported in Kaupmann et al., 1997, Nature 386:239-246. In particular embodiments, GABA_{B}R1b is rat GABA_{B}R1b and has the amino acid sequence reported in Kaupmann et al., 1997, Nature 386:239-246. In particular embodiments, GABA_{B}R1a is human GABA_{B}R1a and has an amino acid sequence selected from the group consisting of: SEQ.ID.NO.:21 and the protein encoded by SEQ.ID.NO.:23.

In particular embodiments, the functional response is selected from the group consisting of: changes in pigment distribution in melanophore cells; changes in cAMP or calcium concentration; and changes in membrane currents in Xenopus oocytes. In particular embodiments, the change in pigment distribution is pigment aggregation; the change in cAMP concentration is a decrease in cAMP concentration; the change in membrane current is the modulation of an inwardly rectifying potassium current.

In a particular embodiment of the above-described method, the cells are eukaryotic cells. In another embodiment; the cells are mammalian cells. In other embodiments, the cells are L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL, 1651), CHO-K1 (ATCC CCL 61), 3T3 (ATCC CCL 92), NIH13T3 (ATCC CRL 1658), HeLa (ATCC CCL 2), C 127I (ATCC CRL 1616), BS-C-1 (ATCC CCL 26), MRC-5 (ATCC CCL 171), Xenopus melanophores, or Xenopus oocytes.

In a particular embodiment of the above-described method, the cells are transfected with separate expression vectors that direct the expression of HG20 and either GABA_{B}R1a or GABA_{B}R1b in the cells. In other embodiments, the cells are transfected with a single expression vector that direct the expression of both HG20 and GABA_{B}R1a or GABA_{B}R1b in the cells.

In a particular embodiment, the cells are Xenopus melanophores and the functional response is pigment aggregation. In another embodiment, the cells are HEK293 cells and the functional response is a decrease in cAMP level. In another embodiment, the cells are Xenopus oocytes and the functional response is the production of an inwardly rectifying potassium current.

The conditions under which step (b) of the method is practiced are conditions that are typically used in the art for the study of protein-ligand interactions: e.g., physiological pH; salt conditions such as those represented by such commonly used buffers as PBS or in tissue culture media; a temperature of about 4° C. to about 55° C.

The above-described assay can be easily modified to form a method to identify antagonists of the GABA_{B} receptor. Such a method comprises:
(a) providing cells by transfecting cells with:
   (1) an expression vector that directs the expression of HG20 in the cells; and
   (2) an expression vector that directs the expression of GABA_{B}R1a or GABA_{B}R1b in the cells;
(b) exposing the cells to a substance that is a known agonist of the GABA_{B} receptor;
(c) measuring the amount of a functional response of the cells that have been exposed to the known agonist;
(d) exposing the cells concurrently to the known agonist and to a substance that is suspected of being an antagonist of the GABA_{B} receptor;
(e) measuring the amount of a functional response of the cells that have been exposed to the substance and the known agonist;
(f) comparing the amount of the functional response measured in step (c) with the amount of the functional response measured in step (e);
wherein if the amount of the functional response measured in step (c) is greater than the amount of the functional response measured in step (e), the substance is an antagonist of the GABA_{B} receptor.

Additional types of functional assays that can be used to identify agonists and antagonists of GABA_{B} receptors include transcription-based assays. Transcription-based assays involve the use of a reporter gene whose transcription is driven by an inducible promoter whose activity is regulated by a particular intracellular event such as, e.g., changes in intracellular calcium levels that are caused by the interaction of a receptor with a ligand. Transcription-based assays are reviewed in Rutter et al., 1998, Chemistry & Biology 5:R285-R290.

The transcription-based assays of the present invention rely on the expression of reporter genes whose transcription is activated or repressed as a result of intracellular events that are caused by the interaction of an agonist with a heterodimer of HG20 and either GABA_{B}R1a or GABA_{B}R1b where the heterodimer forms a functional GABA_{B} receptor.

An extremely sensitive transcription based assay is disclosed in Zlokarnik et al., 1998, Science 279:84-88 (Zlokarnik) and also in U.S. Pat. No. 5,741,657. The assay disclosed in Zlokarnik and U.S. Pat. No. 5,741,657 employs a plasmid encoding β-lactamase under the control of an inducible promoter. This plasmid is transfected into cells together with a plasmid encoding a receptor for which it is desired to identify agonists. The inducible promoter on the β-lactamase is chosen so that it responds to at least one intracellular signal that is generated when an agonist binds to the receptor. Thus, following such binding of agonist to receptor, the level of β-lactamase in the transfected cells increases. This increase in β-lactamase is made measurable by treating the cells with a cell-permeable dye that is a substrate for β-lactamase. The dye contains two fluorescent moieties. In the intact dye, the two fluorescent moieties are close enough to one another that fluorescent resonance energy transfer (FRET) can take place between them. Following cleavage of the dye into two parts by β-lactamase, the two fluorescent moieties are located on different parts, and thus can drift apart. This increases the distance between the fluorescent moieties, thus decreasing the amount of FRET that can occur between them. It is this decrease in FRET that is measured in the assay.

One skilled in the art can modify the assay described in Zlokarnik and U.S. Pat. No. 5,741,657 to form an assay for identifying agonists of GABA_{B} receptors by using an inducible promoter to drive β-lactamase that is activated by an intracellular signal generated by the interaction of agonists and the GABA_{B} receptor. To produce the GABA_{B} receptor, a plasmid encoding HG20 and a plasmid encoding GABA_{B}R1a or GABA_{B}R1b would be transfected into the cells. The cells would be exposed to the cell-permeable dye and then exposed to substances suspected of being agonists of the GABA_{B} receptor. Those substances that cause a decrease in FRET are likely to actually be agonists of the GABA_{B} receptor.

Accordingly, the present invention includes a method for identifying agonists of the GABA_{B} receptor comprising:
(a) transfecting cells with:
   (1) an expression vector that directs the expression of HG20 in the cells;
   (2) an expression vector that directs the expression of GABA_{B}R1a or GABA_{B}R1b in the cells;
   (3) an expression vector that directs the expression of lactamase under the control of an inducible promoter that is activated by an intracellular signal generated by the interaction of agonists and the GABA_{B} receptor;
(b) exposing the cells to a substrate of β-lactamase that is a cell-permeable dye that contains two fluorescent moieties where the two fluorescent moieties are on different parts of the dye and cleavage of the dye by β-lactamase allows the two fluorescent moieties to drift apart;
(c) measuring the amount of fluorescent resonance energy transfer (FRET) in the cells in the absence of the substance of step (d);
(d) exposing the cells to a substance that is suspected of being an agonist of the GABA_{B} receptor;
(e) measuring the amount of FRET in the cells after exposure of the cells to the substance;
wherein if the amount of FRET in the cells measured in step (e) is less that the amount of FRET measured in the cells in step (c), then the substance is an agonist of the GABA_{B} receptor.

Substeps (1)-(3) of step (a) can be practiced in any order.

The assay described above can be modified to an assay for identifying antagonists of the GABA_{B} receptor. Such modification would involve the use of β-lactamase under the control of a promoter that is repressed by at least one intracellular signal generated by interaction of an agonist with the GABA_{B} receptor and would also involve running the assay in the presence of a known agonist. When the cells are exposed to substances suspected of being antagonists of the GABA_{B} receptor, β-lactamase will be induced, and FRET will decrease, only if the substance tested is able to counteract the effect of the agonist, i.e., only if the substance tested is actually an antagonist.

Accordingly, the present invention includes a method for identifying antagonists of the GABA_{B} receptor comprising:
(a) transfecting cells with:
   (1) an expression vector that directs the expression of HG20 in the cells;
   (2) an expression vector that directs the expression of GABA_{B}R1a or GABA_{B}R1b in the cells;
   (3) an expression vector that directs the expression of α-lactamase under the control of an inducible promoter that is repressed by at least one intracellular signal generated by interaction of an agonist with the GABA_{B} receptor;
(b) exposing the cells to a known agonist of the GABA_{B} receptor;
(c) exposing the cells to a substrate of β-lactamase that is a cell-permeable dye that contains two fluorescent moieties where the two fluorescent moieties are on different parts of the dye and cleavage of the dye by β-lactamase allows the two fluorescent moieties to drift apart;
(d) measuring the amount of fluorescent resonance energy transfer (FRET) in the cells in the absence of the substance of step (e);
(e) exposing the cells to a substance that is suspected of being an antagonist of the GABA_{B} receptor;
(f) measuring the amount of FRET in the cells after exposure of the cells to the substance;
wherein if the amount of FRET in the cells measured in step (f) is less that the amount of FRET measured in the cells in step (d), then the substance is an antagonist of the GABA_{B} receptor.

Substeps (1)-(3) of step (a) can be practiced in any order.

In particular embodiments of the assays employing β-lactamase described above, the cells are eukaryotic cells. In particular embodiments, the cells are mammalian cells. In particular embodiments, the cells are selected from the group consisting of: L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171), Xenopus melanophores, and Xenopus oocytes.

In other embodiments, the inducible promoter that is repressed by at least one intracellular signal generated by interaction of an agonist with the GABA_{B} receptor is a promoter that is repressed by decreases in cAMP levels or changes in potassium currents.

In other embodiments, the inducible promoter that is activated by at least one intracellular signal generated by interaction of an agonist with the GABA_{B} receptor is a promoter that is activated by decreases in cAMP levels or changes in potassium currents.

In other embodiments, the known agonist is selected from the group consisting of: GABA, saclofen, (−)baclofen, glycine, and (L)-glutamic acid.

In other embodiments, β-lactamase is TEM-1 β-lactamase from Escherichia coli.

In other embodiments, the substrate of α-lactamase is CCF2/AM (Zlokarnik et al., 1998, Science 279:84-88).

In other embodiments, HG20 has an amino acid sequence of SEQ.ID.NO.:2.

In other embodiments of the above-described methods, HG20 comprises an amino acid sequence selected from the group consisting of:
SEQ.ID.NO.:2;
Positions 9-941 of SEQ.ID.NO.:2;
Positions 35-941 of SEQ.ID.NO.:2;
Positions 36-941 of SEQ.ID.NO.:2;
Positions 38-941 of SEQ.ID.NO.:2;
Positions 39-941 of SEQ.ID.NO.:2;
Positions 42-941 of SEQ.ID.NO.:2;
Positions 44-941 of SEQ.ID.NO.:2;
Positions 46-941 of SEQ.ID.NO.:2;
Positions 52-941 of SEQ.ID.NO.:2; and
Positions 57-941 of SEQ.ID.NO.:2.

In other embodiments, GABA_{B}R1a is murine GABA_{B}R1a and has the amino acid sequence SEQ.ID.NO.:20. In other embodiments, GABA_{B}R1a is rat GABA_{B}R1a and has the amino acid sequence reported in Kaupmann et al., 1997, Nature 386:239-246. In other embodiments, GABA_{B}R1b is rat GABA_{B}R1b and has the amino acid sequence reported in Kaupmann et al., 1997, Nature 386:239-246. In other embodiments, GABA_{B}R1a is human GABA_{B}R1a and has an amino acid sequence selected from the group consisting of: SEQ.ID.NO.:21 and the protein encoded by SEQ.ID.NO.:23.

In particular embodiments, the cells express a promiscuous G-protein, e.g., Gα15 or Gα16.

In particular embodiments, the inducible promoter is a promoter that is activated or repressed by NF-κB or NFAT.

The assays described above could be modified to identify inverse agonists. In such assays, one would expect a decrease in β-lactamase activity. Similarly, inverse agonists can be identified by modifying the functional assays that were described previously where those functional assays monitored decreases in cAMP levels. In the case of assays for inverse agonists, increases in cAMP levels would be observed.

Other transcription-based assays that can be used to identify agonists and antagonists of the GABA_{B} receptor rely on the use of green fluorescent proteins or luciferase as reported genes. An example of such an assay comprises:
(a) transfecting cells with:
   (1) an expression vector that directs the expression of HG20 in the cells;
   (2) an expression vector that directs the expression of GABA_{B}R1a or GABA_{B}R1b in the cells;
   (3) an expression vector that directs the expression of green fluorescent protein (GFP) under the control of an inducible promoter that is activated by an intracellular signal generated by the interaction of agonists and the GABA_{B} receptor;
(b) measuring the amount of fluorescence from GFP in the cells;
(c) exposing the cells to a substance that is suspected of being an agonist of the GABA_{B} receptor;
(d) measuring the amount of fluorescence from GFP in the cells that have been exposed to the substance;
wherein if the amount of fluorescence from GFP in the cells measured in step (b) is less that the amount of fluorescence from GFP measured in the cells in step (d), then the substance is an agonist of the GABA_{B} receptor.

The present invention also includes assays for the identification of agonists or antagonists of GABA_{B} receptors that are based upon FRET between a first and a second fluorescent dye where the first dye is bound to one side of the plasma membrane of a cell expressing a heterodimer of HG20 and GABA_{B}R1a or GABA_{B}R1b and the second dye is free to shuttle from one face of the membrane to the other face in response to changes in membrane potential. In certain embodiments, the first dye is impenetrable to the plasma membrane of the cells and is bound predominately to the extracellular surface of the plasma membrane. The second dye is trapped within the plasma membrane but is free to diffuse within the membrane. At normal (i.e., negative) resting potentials of the membrane, the second dye is bound predominately to the inner surface of the extracellular face of the plasma membrane, thus placing the second dye in close proximity to the first dye. This close proximity allows for the generation of a large amount of FRET between the two dyes. Following membrane depolarization, the second dye moves from the extracellular face of the membrane to the intracellular face, thus increasing the distance between the dyes. This increased distance results in a decrease in FRET, with a corresponding increase in fluorescent emission derived from the first dye and a corresponding decrease in the fluorescent emission from the second dye. See FIG. 1 of Gonzalez & Tsien, 1997, Chemistry & Biology 4:269-277. See also González & Tsien, 1995, Biophys. J. 69:1272-1280 and U.S. Pat. No. 5,661,035.

In certain embodiments, the first dye is a fluorescent lectin or a fluorescent phospholipid that acts as the fluorescent donor. Examples of such a first dye are: a coumarin-labeled phosphatidylethanolamine (e.g., N-(6-chloro-7-hydroxy-2-oxo-2H-1-benzopyran-3-carboxamidoacetyl)-dimyristoylphosphatidyl-ethanolamine) or N-(7-nitrobenz-2-oxa-1,3-diazol-4-yl)-dipalmitoylphosphatidylethanolamine); a fluorescently-labeled lectin (e.g., fluorescein-labeled wheat germ agglutinin). In certain embodiments, the second dye is an oxonol that acts as the fluorescent acceptor. Examples of such a second dye are: bis(1,3-dialkyl-2-thiobarbiturate)trimethineoxonols (e.g., bis(1,3-dihexyl-2-thiobarbiturate)trimethineoxonol) or pentamethineoxonol analogues (e.g., bis(1,3-dihexyl-2-thiobarbiturate)pentamethineoxonol; or bis(1,3-dibutyl-2-thiobarbiturate)pentamethineoxonol). See González & Tsien, 1997, Chemistry & Biology 4:269-277 for methods of synthesizing various dyes suitable for use in the present invention. In certain embodiments, the assay may comprise a natural carotenoid, e.g., astaxanthin, in order to reduce photodynamic damage due to singlet oxygen.

Accordingly, the present invention provides a method of identifying agonists of GABA_{B} receptors comprising:
(a) providing test cells comprising:
   (1) an expression vector that directs the expression of HG20 in the cells;
   (2) an expression vector that directs the expression of GABA_{B}R1a or GABA_{B}R1b in the cells;
   (3) an expression vector that directs the expression of an inwardly rectifying potassium channel;
   (4) a first fluorescent dye, where the first dye is bound to one side of the plasma membrane; and
   (5) a second fluorescent dye, where the second fluorescent dye is free to shuttle from one face of the plasma membrane to the other face in response to changes in membrane potential;
(b) exposing the test cells to a substance that is suspected of being an agonist of the GABA_{B} receptor;
(c) measuring the amount of fluorescence resonance energy transfer (FRET) in the test cells that have been exposed to the substance;
(d) comparing the amount of FRET exhibited by the test cells that have been exposed to the substance with the amount of FRET exhibited by control cells;
wherein if the amount of FRET exhibited by the test cells is less than the amount of FRET exhibited by the control cells, the substance is an agonist of the GABA_{B} receptor;
where the control cells are either (1) cells that are essentially the same as the test cells except that they do not comprise at least one of the items listed at (a)
(1)-(5) but have been exposed to the substance; or (2) test cells that have not been exposed to the substance.

The above-described assay can be easily modified to form a method to identify antagonists of the GABA_{B} receptor. Such a method comprises:
(a) providing test cells comprising:
   (1) an expression vector that directs the expression of HG20 in the cells;
   (2) an expression vector that directs the expression of GABA_{B}R1a or GABA_{B}R1b in the cells;
   (3) an expression vector that directs the expression of an inwardly rectifying potassium channel;
   (4) a first fluorescent dye, where the first dye is bound to one side of the plasma membrane; and
   (5) a second fluorescent dye, where the second fluorescent dye is free to shuttle from one face of the plasma membrane to the other face in response to changes in membrane potential;
(b) exposing the test cells to a known agonist of the GABA_{B} receptor in the presence of a substance that is suspected of being an antagonist of the GABA_{B} receptor;
(c) exposing the test cells to the known agonist of the GABA_{B} receptor in the absence of the substance that is suspected of being an antagonist of the GABA_{B} receptor;
(d) measuring the amount of fluorescence resonance energy transfer (FRET) in the test cells of steps (b) and (c);
(e) comparing the amount of FRET exhibited by the test cells of steps (b) and (c);
where if the amount of FRET exhibited by the test cells of step (b) is greater than the amount of FRET exhibited by the test cells of step (c), the substance is an antagonist of the GABA_{B} receptor.

In particular embodiments of the above-described methods, the expression vectors are transfected into the test cells.

In particular embodiments of the above-described methods, HG20 has an amino acid sequence of SEQ.ID.NO.:2.

In particular embodiments of the above-described methods, HG20 comprises an amino acid sequence selected from the group consisting of:
SEQ.ID.NO.:2;
Positions 9-941 of SEQ.ID.NO.:2;
Positions 35-941 of SEQ.ID.NO.:2;
Positions 36-941 of SEQ.ID.NO.:2;
Positions 38-941 of SEQ.ID.NO.:2;
Positions 39-941 of SEQ.ID.NO.:2;
Positions 42-941 of SEQ.ID.NO.:2;
Positions 44-941 of SEQ.ID.NO.:2;
Positions 46-941 of SEQ.ID.NO.:2;
Positions 52-941 of SEQ.ID.NO.:2; and
Positions 57-941 of SEQ.ID.NO.:2.

In particular embodiments of the above-described methods, GABA_{B}R1a is murine GABA_{B}R1a and has the amino acid sequence SEQ.ID.NO.:20. In particular embodiments, GABA_{B}R1a is rat GABA_{B}R1a and has the amino acid sequence reported in Kaupmann et al., 1997, Nature 386:239-246. In particular embodiments, GABA_{B}R1b is rat GABA_{B}R1b and has the amino acid sequence reported in Kaupmann et al., 1997, Nature 386:239-246. In particular embodiments, GABA_{B}R1a is human GABA_{B}R1a and has an amino acid sequence selected from the group consisting of: SEQ.ID.NO.:21 and the protein encoded by SEQ.ID.NO.:23.

Inwardly rectifying potassium channels that are suitable for use in the methods of the present invention are disclosed in, e.g., Misgeld et al., 1995, Prog. Neurobiol. 46:423-462; North, 1989, Br. J. Pharmacol. 98:13-23; Gahwiler et al., 1985, Proc. Natl. Acad. Sci USA 82:1558-1562; Andrade et al., 1986, Science 234:1261.

In particular embodiments of the above-described methods, the first fluorescent dye is selected from the group consisting of: a fluorescent lectin; a fluorescent phospholipid; a coumarin-labeled phosphatidylethanolamine; N-(6-chloro-7-hydroxy-2-oxo-2H-1-benzopyran-3-carboxamidoacetyl)-dimyristoylphosphatidyl-ethanolamine); N-(7-nitrobenz-2-oxa-1,3-diazol-4-yl)-dipalmitoylphosphatidylethanolamine); and fluorescein-labeled wheat germ agglutinin.

In particular embodiments of the above-described methods, the second fluorescent dye is selected from the group consisting of: an oxonol that acts as the fluorescent acceptor; bis(1,3-dialkyl-2-thiobarbiturate)trimethineoxonols; bis(1,3-dihexyl-2-thiobarbiturate)trimethineoxonol; bis(1,3-dialkyl-2-thiobarbiturate)quatramethineoxonols; bis(1,3-dialkyl-2-thiobarbiturate)pentamethineoxonols; bis(1,3-dihexyl-2-thiobarbiturate)pentamethineoxonol; bis(1,3-dibutyl-2-thiobarbiturate)pentamethineoxonol); and bis(1,3-dialkyl-2-thiobarbiturate)hexamethineoxonols.

In a particular embodiment of the above-described methods, the cells are eukaryotic cells. In another embodiment, the cells are mammalian cells. In other embodiments, the cells are L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171), Xenopus melanophores, or Xenopus oocytes.

In a particular embodiment of the above-described methods, the cells are transfected with separate expression vectors that direct the expression of HG20 and either GABA_{B}R1a or GABA_{B}R1b in the cells. In other embodiments, the cells are transfected with a single expression vector that direct the expression of both HG20 and GABA_{B}R1a or GABA_{B}R1b in the cells.

The conditions under which step (b) of the first method described above and steps (b) and (c) of the second method described above are practiced are conditions that are typically used in the art for the study of protein-ligand interactions: e.g., physiological pH; salt conditions such as those represented by such commonly used buffers as PBS or in tissue culture media; a temperature of about 4° C. to about 55° C.

The GABA_{B} receptor belongs to the class of proteins known as G-protein coupled receptors (GPCRs). GPCRs transmit signals across cell membranes upon the binding of ligand. The ligand-bound GPCR interacts with a heterotrimeric G-protein, causing the Gα subunit of the G-protein to disassociate from the Gβ and Gγ subunits. The Gα subunit can then go on to activate a variety of second messenger systems.

Generally, a particular GPCR is only coupled to a particular type of G-protein. Thus, to observe a functional response from the GPCR, it is necessary to ensure that the proper G-protein is present in the system containing the GPCR. It has been found, however, that there are certain G-proteins that are “promiscuous.” These promiscuous G-proteins will couple to, and thus transduce a functional signal from, virtually any GPCR. See Offermanns & Simon, 1995, J. Biol. Chem. 270:15175, 15180 (Offermanns). Offermanns described a system in which cells are transfected with expression vectors that result in the expression of one of a large number of GPCRs as well as the expression of one of the promiscuous G-proteins Gα15 or Gα16. Upon the addition of an agonist of the GPCR to the transfected cells, the GPCR was activated and was able, via Gα15 or Gα16, to activate the β isoform of phospholipase C, leading to an increase in inositol phosphate levels in the cells.

Therefore, by making use of these promiscuous G-proteins as in Offermanns, it is possible to set up functional assays for the GABA_{B} receptor, even in the absence of knowledge of the G-protein with which the GABA_{B} receptors coupled in vivo. One possibility for utilizing promiscuous G-proteins in connection with the GABA_{B} receptor includes a method of identifying agonists of the GABA_{B} receptor comprising:
(a) providing cells that express HG20, GABA_{B}R1a or GABA_{B}R1b, and a promiscuous G-protein, where HG20 and either GABA_{B}R1a or GABA_{B}R1b form a heterodimer representing a functional GABA_{B} receptor;
(b) exposing the cells to a substance that is a suspected agonist of the GABA_{B} receptor;
(c) measuring the level of inositol phosphates in the cells;
where an increase in the level of inositol phosphates in the cells as compared to the level of inositol phosphates in the cells in the absence of the suspected agonist indicates that the substance is an agonist of the GABA_{B} receptor.

Levels of inositol phosphates can be measured by monitoring calcium mobilization. Intracellular calcium mobilization is typically assayed in whole cells under a microscope using fluorescent dyes or in cell suspensions via luminescence using the aequorin assay.

In methods related to those described above, rather than using changes in inositol phosphate levels as an indication of GABA_{B} receptor function, potassium currents are measured. This is feasible since the GABA_{B} receptor, like other metabotropic receptors, is expected to be coupled to potassium channels. Thus, one could measure GABA_{B} receptor coupling to GIRK2 channels or to other potassium channels in oocytes.

In a particular embodiment of the above-described method, the cells are eukaryotic cells. In another embodiment, the cells are mammalian cells. In other embodiments, the cells are L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171), or Xenopus oocytes.

In a particular embodiment of the above-described method, the cells are transfected with expression vectors that direct the expression of HG20, GABA_{B}R1a or GABA_{B}R1b, and the promiscuous G-protein in the cells.

The conditions under which step (b) of the method is practiced are conditions that are typically used in the art for the study of protein-ligand interactions: e.g., physiological pH; salt conditions such as those represented by such commonly used buffers as PBS or in tissue culture media; a temperature of about 4° C. to about 55° C.

In a particular embodiment of the above-described method, the promiscuous G-protein is selected from the group consisting of Gα15 or Gα16. Expression vectors containing Gα15 or Gα16 are known in the art. See, e.g., Offermanns; Buhl et al., 1993, FEBS Lett. 323:132-134; Amatruda et al., 1993, J. Biol. Chem. 268:10139-10144.

The above-described assay can be easily modified to form a method to identify antagonists of the GABA_{B} receptor. Such a method is also part of the present invention and comprises:
(a) providing cells that express HG20, GABA_{B}R1a or GABA_{B}R1b, and a promiscuous G-protein;
(b) exposing the cells to a substance that is an agonist of the GABA_{B} receptor;
(c) subsequently or concurrently to step (b), exposing the cells to a substance that is a suspected antagonist of the GABA_{B} receptor;
(d) measuring the level of inositol phosphates in the cells;
where a decrease in the level of inositol phosphates in the cells in the presence of the suspected antagonist as compared to the level of inositol phosphates in the cells in the absence of the suspected antagonist indicates that the substance is an antagonist of the GABA_{B} receptor.

In a particular embodiment of the above-described method, the agonist is an amino acid such as GABA, glutamate, glycine, or amino acid analogues such as (−)baclofen.

In a particular embodiment of the above-described method, the cells are eukaryotic cells. In another embodiment, the cells are mammalian cells. In other embodiments, the cells are L cells L-M(TK⁻) (ATCC CCL 1.3), L cells L-M (ATCC CCL 1.2), HEK293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171), or Xenopus oocytes.

The conditions under which steps (b) and (c) of the method are practiced are conditions that are typically used in the art for the study of protein-ligand interactions: e.g., physiological pH; salt conditions such as those represented by such commonly used buffers as PBS or in tissue culture media; a temperature of about 4° C. to about 55° C.

In a particular embodiment of the above-described method, the cells are transfected with expression vectors that direct the expression of HG20, GABA_{B}R1a or GABA_{B}R1b, and the promiscuous G-protein in the cells.

In a particular embodiment of the above-described method, the promiscuous G-protein is selected from the group consisting of Gα15 or Gα16.

In particular embodiments of the above-described methods, HG20 has an amino acid sequence of SEQ.ID.NO.:2.

In other embodiments of the above-described methods, HG20 comprises an amino acid sequence selected from the group consisting of:
SEQ.ID.NO.:2;
Positions 9-941 of SEQ.ID.NO.:2;
Positions 35-941 of SEQ.ID.NO.:2;
Positions 36-941 of SEQ.ID.NO.:2;
Positions 38-941 of SEQ.ID.NO.:2;
Positions 39-941 of SEQ.ID.NO.:2;
Positions 42-941 of SEQ.ID.NO.:2;
Positions 44-941 of SEQ.ID.NO.:2;
Positions 46-941 of SEQ.ID.NO.:2;
Positions 52-941 of SEQ.ID.NO.:2; and
Positions 57-941 of SEQ.ID.NO.:2.

In other embodiments, GABA_{B}R1a is murine GABA_{B}R1a and has the amino acid sequence SEQ.ID.NO.:20. In other embodiments, GABA_{B}R1a is rat GABA_{B}R1a and has the amino acid sequence reported in Kaupmann et al., 1997, Nature 386:239-246. In other embodiments, GABA_{B}R1b is rat GABA_{B}R1b and has the amino acid sequence reported in Kaupmann et al., 1997, Nature 386:239-246. In other embodiments, GABA_{B}R1a is human GABA_{B}R1a and has an amino acid sequence selected from the group consisting of: SEQ.ID.NO.:21 and the protein encoded by SEQ.ID.NO.:23.

While the above-described methods are explicitly directed to testing whether “a” substance is an agonist or antagonist of the GABA_{B} receptor, it will be clear to one skilled in the art that such methods can be adapted to test collections of substances, e.g., combinatorial libraries, to determine whether any members of such collections are activators or inhibitors of the GABA_{B} receptor. Accordingly, the use of collections of substances, or individual members of such collections, as the substance in the above-described methods is within the scope of the present invention.

The present invention includes pharmaceutical compositions comprising agonists and antagonists of GABA_{B} receptors that have been identified by the above-described methods. The agonists and antagonists are generally combined with pharmaceutically acceptable carriers to form pharmaceutical compositions. Examples of such carriers and methods of formulation of pharmaceutical compositions containing agonists and antagonists and carriers can be found in Remington's Pharmaceutical Sciences. To form a pharmaceutically acceptable composition suitable for effective administration, such compositions will contain a therapeutically effective amount of the agonists and antagonists.

Therapeutic or prophylactic compositions are administered to an individual in amounts sufficient to treat or prevent conditions where GABA_{B} receptor activity is abnormal. The effective amount can vary according to a variety of factors such as the individual's condition, weight, gender, and age. Other factors include the mode of administration. The appropriate amount can be determined by a skilled physician.

Compositions can be used alone at appropriate dosages. Alternatively, co-administration or sequential administration of other agents can be desirable.

The compositions can be administered in a wide variety of therapeutic dosage forms in conventional vehicles for administration. For example, the compositions can be administered in such oral dosage forms as tablets, capsules (each including timed release and sustained release formulations), pills, powders, granules, elixirs, tinctures, solutions, suspensions, syrups and emulsions, or by injection. Likewise, they can also be administered in intravenous (both bolus and infusion), intraperitoneal, subcutaneous, topical with or without occlusion, or intramuscular form, all using forms well known to those of ordinary skill in the pharmaceutical arts.

Advantageously, compositions can be administered in a single daily dose, or the total daily dosage can be administered in divided doses of two, three or four times daily. Furthermore, compositions can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

The dosage regimen utilizing the compositions is selected in accordance with a variety of factors including type, species, age, weight, sex and medical condition of the patient; the severity of the condition to be treated; the route of administration; the renal, hepatic and cardiovascular function of the patient; and the particular composition thereof employed. A physician of ordinary skill can readily determine and prescribe the effective amount of the composition required to prevent, counter or arrest the progress of the condition. Optimal precision in achieving concentrations of composition within the range that yields efficacy without toxicity requires a regimen based on the kinetics of the composition's availability to target sites. This involves a consideration of the distribution, equilibrium, and elimination of a composition.

Agonists and antagonists identified by the above-described methods are useful in the same manner as well-known agonists and antagonists of other GABA_{B} receptors. For example, (−) baclofen is a known agonist of GABA_{B} receptors and, in racemic form, is a clinically useful muscle relaxant known as LIORESAL® (Bowery & Pratt, 1992, Arzneim.-Forsch./Drug Res. 42:215-223 [Bowery & Pratt]). Similarly, the agonists and antagonists of GABA_{B} receptors identified by the methods of the present invention are expected to be useful as muscle relaxants. Bowery & Pratt, at Table 1, page 219, list the therapeutic potential of GABA_{B} receptor agonists and antagonists. For agonists, the therapeutic potential is said to include use as muscle relaxants and anti-asthmatics. For antagonists, the therapeutic potential is said to include use as antidepressants, anticonvulsants, nootropics, and anxiolytics. Additionally, at page 220, left column, Bowery & Pratt list some additional therapeutic uses for the GABA_{B} receptor agonist (−) baclofen: treatment of trigeminal neuralgia and reversal of ethanol withdrawal symptoms. Given the wide range of utility displayed by known agonists and antagonists of GABA_{B} receptors, it is clear that those skilled in the art would consider the agonists and antagonists identified by the methods of the present invention to be pharmacologically useful. In addition, it is believed that such agonists and antagonists will also be useful in the treatment of epilepsy, neuropsychiatric disorders, and dementias.

When screening compounds in order to identify potential pharmaceuticals that specifically interact with a target receptor, it is necessary to ensure that the compounds identified are as specific as possible for the target receptor. To do this, it is necessary to screen the compounds against as wide an array as possible of receptors that are similar to the target receptor. Thus, in order to find compounds that are potential pharmaceuticals that interact with receptor A, it is necessary not only to ensure that the compounds interact with receptor A (the “plus target”) and produce the desired pharmacological effect through receptor A, it is also necessary to determine that the compounds do not interact with receptors B, C, D, etc (the “minus targets”). In general, as part of a screening program, it is important to have as many minus targets as possible (see Hodgson, 1992, Bio/Technology 10:973-980, at 980). HG20 protein, DNA encoding HG20 protein, GABA_{B}R1a protein, DNA encoding GABA_{B}R1a protein, and recombinant cells that have been engineered to express HG20 protein and GABA_{B}R1a protein have utility in that they can be used as “minus targets” in screens design to identify compounds that specifically interact with other G-protein coupled receptors, i.e., non-GABA_{B} receptors.

The present invention also includes antibodies to the HG20 protein. Such antibodies may be polyclonal antibodies or monoclonal antibodies. The antibodies of the present invention are raised against the entire HG20 protein or against suitable antigenic fragments of the protein that are coupled to suitable carriers, e.g., serum albumin or keyhole limpet hemocyanin, by methods well known in the art. Methods of identifying suitable antigenic fragments of a protein are known in the art. See, e.g., Hopp & Woods, 1981, Proc. Natl. Acad. Sci. USA 78:3824-3828; and Jameson & Wolf, 1988, CABIOS (Computer Applications in the Biosciences) 4:181-186. Particularly suitable peptides are: amino acids 357-371 of SEQ.ID.NO.:2 and amino acids 495-511 of SEQ.ID.NO.:2. Also, anti-peptide antisera can be generated by immunization of New Zealand White rabbits with a KLH-conjugation of a 20 amino acid synthetic peptide corresponding to residues 283-302 of HG20 (GWYEPSWWEQVHTEANSSRC) (a portion of SEQ.ID.NO.:2).

For the production of polyclonal antibodies, HG20 protein or an antigenic fragment, coupled to a suitable carrier, is injected on a periodic basis into an appropriate non-human host animal such as, e.g., rabbits, sheep, goats, rats, mice. The animals are bled periodically and sera obtained are tested for the presence of antibodies to the injected antigen. The injections can be intramuscular, intraperitoneal, subcutaneous, and the like, and can be accompanied with adjuvant.

For the production of monoclonal antibodies, HG20 protein or an antigenic fragment, coupled to a suitable carrier, is injected into an appropriate non-human host animal as above for the production of polyclonal antibodies. In the case of monoclonal antibodies, the animal is generally a mouse. The animal's spleen cells are then immortalized, often by fusion with a myeloma cell, as described in Kohler & Milstein, 1975, Nature 256:495-497. For a fuller description of the production of monoclonal antibodies, see Antibodies: A Laboratorv Manual, Harlow & Lane, eds., Cold Spring Harbor Laboratory Press, 1988.

Gene therapy may be used to introduce HG20 polypeptides into the cells of target organs. Nucleotides encoding HG20 polypeptides can be ligated into viral vectors which mediate transfer of the nucleotides by infection of recipient cells. Suitable viral vectors include retrovirus, adenovirus, adeno-associated virus, herpes virus, vaccinia virus, and polio virus based vectors. Alternatively, nucleotides encoding HG20 polypeptides can be transferred into cells for gene therapy by non-viral techniques including receptor-mediated targeted transfer using ligand-nucleotide conjugates, lipofection, membrane fusion, or direct microinjection. These procedures and variations thereof are suitable for ex vivo as well as in vivo gene therapy. Gene therapy with HG20 polypeptides will be particularly useful for the treatment of diseases where it is beneficial to elevate HG20 activity.

The following non-limiting examples are presented to better illustrate the invention.

### EXAMPLE 1

Cloning and Sequencing of HG20

A cDNA fragment encoding full-length HG20 can be isolated from a human fetal brain cDNA library by using the polymerase chain reaction (PCR) employing the following primer pair:

| | |
|---|---|
| HG20.F139 | |
| 5′-CCGTTCTGAGCCGAGCCG-3′ | (SEQ.ID.NO.:3) |
| HG20.R3195 | |
| 5′-TCCGCAGCCAGAGCCGACAG-3′ | (SEQ.ID.NO.:4) |

The above primer pair is meant to be illustrative only. Those skilled in the art would recognize that a large number of primer pairs, based upon SEQ.ID.NO.:1, could also be used.

PCR reactions can be carried out with a variety of thermostable enzymes including but not limited to AmpliTaq, AmpliTaq Gold, Vent polymerase. For AmpliTaq, reactions can be carried out in 10 mM Tris-Cl, pH 8.3, 2.0 mM MgCl2, 200 μM for each dNTP, 50 mM KCl, 0.2 μM for each primer, 10 ng of DNA template, 0.05 units/μl of AmpliTaq. The reactions are heated at 95° C. for 3 minutes and then cycled 35 times using the cycling parameters of 95° C., 20 seconds, 62° C., 20 seconds, 72° C., 3 minutes. In addition to these conditions, a variety of suitable PCR protocols can be found in PCR Primer, A Laboratory Manual, edited by C. W. Dieffenbach and G. S. Dveksler, 1995, Cold Spring Harbor Laboratory Press.

A suitable cDNA library from which a clone encoding HG20 can be isolated would be a random primed fetal brain cDNA library consisting of approximately 4.0 million primary clones constructed in the plasmid vector pBluescript (Stratagene, LaJolla, Calif.). The primary clones of such a library can be subdivided into pools with each pool containing approximately 20,000 clones and each pool can be amplified separately.

By this method, a cDNA fragment (SEQ.ID.NO.:1) encoding an open reading frame of 941 amino acids (SEQ.ID.NO.:2) is obtained. This cDNA fragment can be cloned into a suitable cloning vector or expression vector. For example, the fragment can be cloned into the mammalian expression vector pcDNA3.1 (Invitrogen, San Diego, Calif.). HG20 protein can then be produced by transferring an expression vector containing SEQ.ID.NO.:1 or portions thereof into a suitable host cell and growing the host cell under appropriate conditions. HG20 protein can then be isolated by methods well known in the art.

Alternatively, other cDNA libraries made from human tissues that express HG20 RNA can be used with PCR primers HG20.F139 and HG20.R3195 in order to amplify a cDNA fragment encoding full-length HG20. Suitable cDNA libraries would be those prepared from cortex, cerebellum, testis, ovary, adrenal gland, thyroid, or spinal cord.

As an alternative to the above-described PCR method, a cDNA clone encoding HG20 can be isolated from a cDNA library using as a probe oligonucleotides specific for HG20 and methods well known in the art for screening cDNA libraries with oligonucleotide probes. Such methods are described in, e.g., Sambrook et al., 1989, Molecular Cloning: A Laboratory Manual; Cold Spring Harbor Laboratory, Cold Spring Harbor, N.Y.; Glover, D. M. (ed.), 1985, DNA Cloning: A Practical Approach, MRL Press, Ltd., Oxford, U. K., Vol. I, II. Oligonucleotides that are specific for HG20 and that can be used to screen cDNA libraries are:

| | |
|---|---|
| HG20.F46 | |
| 5′-GGGATGATCATGGCCAGTGC-3′ | (SEQ.ID.NO.:5) |
| HG20.R179 | |
| 5′-GGATCCATCAAGGCCAAAGA-3′ | (SEQ.ID.NO.:6) |
| HG21.F43 | |
| 5′-GCCGCTGTCTCCTTCCTGA-3′ | (SEQ.ID.NO.:7) |
| HG21.R251 | |
| 5′-TTGGTTCACACTGGTGACCGA-3′ | (SEQ.ID.NO.:8) |
| HG20.R123 | |
| 5′-TTCACCTCCCTGCTGTCTTG-3′ | (SEQ.ID.NO.:9) |
| HG20.F1100 | |
| 5′-CAGGCGATTCCAGTTCACTCA-5′ | (SEQ.ID.NO.:10) |
| HG20.F1747 | |
| 5′-GAACCAAGCCAGCACATCCC-3′ | (SEQ.ID.NO.:11) |
| HG20.R54 | |
| 5′-CCTCGCCATACAGAACTCC-3′ | (SEQ.ID.NO.:12) |
| HG20.R75 | |
| 5′-GTGTCATAGAGCCGCAGGTC-3′ | (SEQ.ID.NO.:13) |
| HG20.F139 | |
| 5′-CCGTTCTGAGCCGAGCCG-3′ | (SEQ.ID.NO.:3) |
| HG20.R3195 | |
| 5′-TCCGCAGCCAGAGCCGACAG-3′ | (SEQ.ID.NO.:4) |

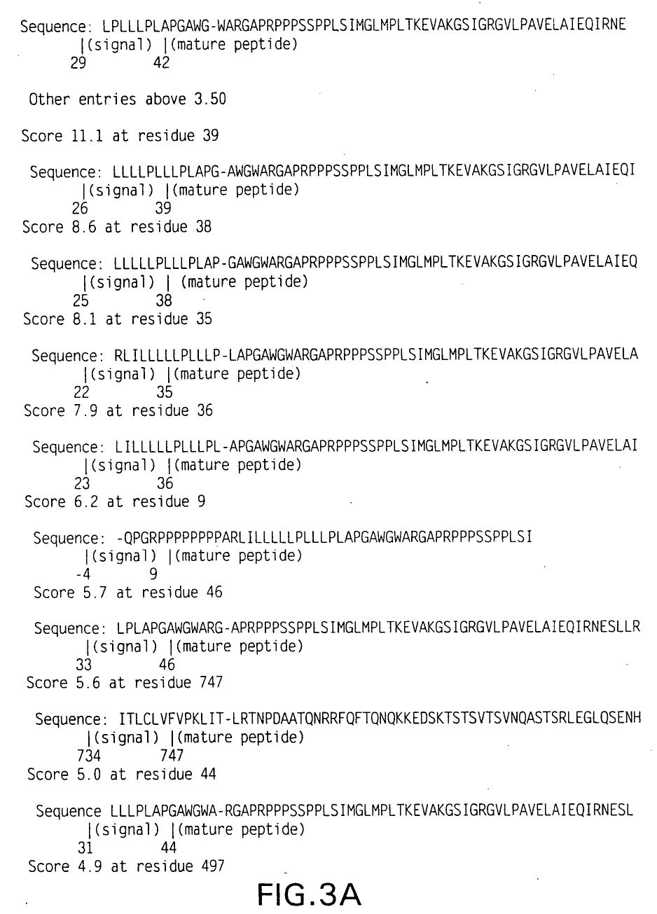

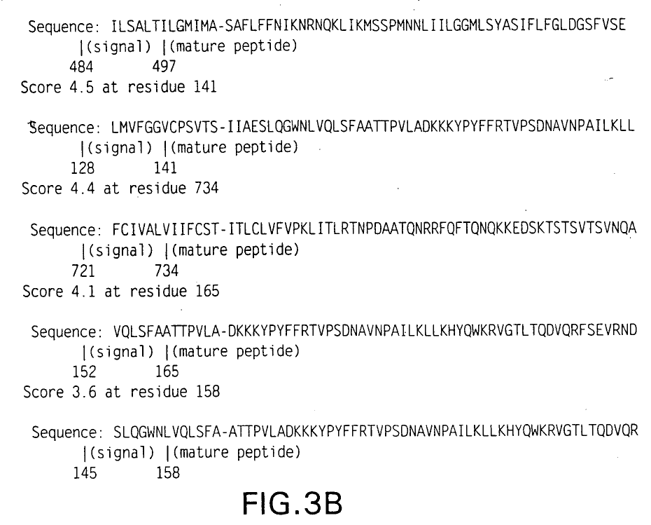

Membrane-spanning proteins, such as GABA_{B} receptors, when first translated generally possess an approximately 16 to 40 amino acid segment known as a signal sequence. Signal sequences direct the nascent protein to be transported through the endoplasmic reticulum membrane, following which signal sequences are cleaved from the protein. Signal sequences generally contain from 4 to 12 hydrophobic residues but otherwise possess little sequence homology. The Protein Analysis tool of the GCG program (Genetics Computer Group, Madison, Wis.), a computer program capable of identifying likely signal sequences, was used to examine the N terminus of HG20. Several likely candidates for cleavage sites which would generate mature HG20 protein, i.e., protein lacking the signal sequence, were identified. The results are shown in FIG. 3.

### EXAMPLE 2

Expression of Hg20 in Normal and Diseased Adrenal Tissue

Northern blots were performed to measure the amount of HG20 RNA in normal and diseased adrenal tissue. The results are shown in Table 2 below. The amount of the approximately 6.5 kb HG20 transcript is shown normalized to the amount of β-actin transcript.

TABLE 2

| | | HG20 | Actin | HG20/ |
|---|---|---|---|---|
| Pathology | Profile | RNA | RNA | actin |
| Pheochromocytoma | M, 30 yr | 0.47 | 0.74 | 0.64 |
| Adrenal carcinoma cortex | M, 69 yr | 0.61 | 0.80 | 0.76 |
| Adrenal adenoma cortex | M, 69 yr | 0.62 | 1.15 | 0.54 |
| Normal Adrenal | M, 26 yr | 1.00 | 1.00 | 1.00 |

The results shown in Table 2 indicate that HG20 expression is decreased in diseased states of the adrenal gland. Thus, increasing the concentration of HG20 in such diseased states is likely to be pharmacologically useful. Accordingly, one skilled in the art would expect agonists of HG20 to be pharmacologically useful.

### EXAMPLE 3

Tissue Distribution of Various HG20 RNA Transcripts

Table 3, below, shows the results of experiments to measure the amount of HG20 RNA transcripts of various lengths in various tissues. The results shown were derived from a multiple tissue Northern blot that was hybridized overnight in expressHyb solution (Clontech). Washing conditions were: 0.1×SSC, 0.1% SDS, at 60° C.

A ³²P-random primer labelled Eco RI fragment containing the full-length native HG20 DNA was used as a hybridization probe. The greater the number of plus signs in a particular tissue, the greater was the amount of HG20 RNA detected in that tissue.

TABLE 3

| Tissue | 6.5 kb | 4.5 kb | 4.0 kb | 1.8 kb |
|---|---|---|---|---|
| cerebellum | ++ | + | | |
| cerebral cortex | ++++ | + | | |
| medulla | + | + | | |
| occipital pole | + | + | | |
| frontal lobe | +++ | + | | |
| temporal lobe | +++ | + | | |
| putamen | ++ | + | | |
| spinal cord n = 2 | ++ | + | | |
| amygdala | +++ | | | |
| caudate nucleus | + | + | | |
| corpus callosum | + | + | | |
| hippocampus | ++ | + | | |
| whole brain | +++ | + | | |
| substantia nigra | + | + | | |
| subthalamic nucleus | + | + | | |
| thalamus | ++ | + | | |
| spleen | | + | | |
| thymus n = 2 | | ++ | | |
| prostate | | ++ | | |
| testis n = 2 | ++ | + | +++ | |
| ovary | | ++ | + | + |
| small intestine n = 2 | | ++ | | |
| colon (mucosal lining) | | ++ | | |
| peripheral blood leucocytes | | ++ | | |
| stomach n = 2 | + | + | | |
| thyroid n = 2 | ++ | ++++ | | |
| lymph node | | + | | |
| trachea | | ++ | | |
| adrenal gland | +++ | +++ | + | ++++ |
| bone marrow | | ++ | | |
| heart | + | ++ | | |
| brain | +++++ | | | |
| placenta | | + | | |
| lung | | + | | |
| liver | | + | | |
| skeletal muscle | + | ++ | | |
| kidney | | + | | |
| pancreas | + | + | | |
| adrenal medulla | +++ | | | + |
| adrenal cortex | +++++ | | ++ | ++ |

The distribution of HG20 RNA shown in Table 3 suggests that HG20 mediates activities of the central and peripheral nervous system.

### EXAMPLE 4

Distribution of HG20 mRNA in Brain

Using in situ hybridisation, the distribution of HG20 mRNA in squirrel monkey brain was studied. Antisense oligonucleotide probes to HG20 were generated on an Applied Biosystems Model 394 DNA synthesizer and purified by preparative polyacrylamide electrophoresis. Probe 1: 5′ATC-TGG-GTT-TGT-TCT-CAG-GGT-GAT-GAG-CTT-CGG-CAC-GAA-TAC-CAG 3′ (SEQ.ID.NO.:14); Probe2: 5′GCT-CTG-TGA-TCT-TCA-TTC-GCA-GGC-GAT-GGT-TTT-CTG-ACT-GTA-GGC 3′ (SEQ.ID.NO.:15). Each oligonucleotide was 3′-end labelled with [³⁵S] deoxyadenosine 5′-(thiotriphosphate) in a 30:1 molar ratio of ³⁵S-isotope:oligonucleotide using terminal deoxynucleotidyl transferase for 15 min at 37° C. in the reaction buffer supplied (Boehringer). Radiolabelled oligonucleotide was separated from unincorporated nucleotides using Sephadex G50 spin columns. The specific activities of the labelled probes in several labelling reactions varied from 1.2-2.3×10⁹ cpm/mg. Squirrel monkey brains were removed and fresh frozen in 1 cm blocks. 12 mm sections were taken and fixed for in situ hybridisation. Hybridisation of the sections was carried out according to the method of Sirinathsinghji et al., 1993, Neuroreports 4:175-178. Briefly, sections were removed from alcohol, air dried and 5×10⁵ cpm of each ³⁵S-labelled probe (both oligonucleotides) in 100 ml of hybridisation buffer was applied to each slide. Labelled “antisense” probe was also used in the presence of an excess (100×) concentration of unlabelled antisense probe to define non-specific hybridisation. Parafilm coverslips were placed over the sections which were incubated overnight (about 16 hr) at 37° C. Following hybridisation the sections were washed for 1 hr at 57° C. in 1×SSC, then rinsed briefly in 0.1×SSC, dehydrated in a series of alcohols, air dried, and exposed to Amersham Hyperfilm bmax X-ray film. Autoradiographs were analyzed using a MCID computerized image analysis system (Image Research Inc., Ontario, Canada).

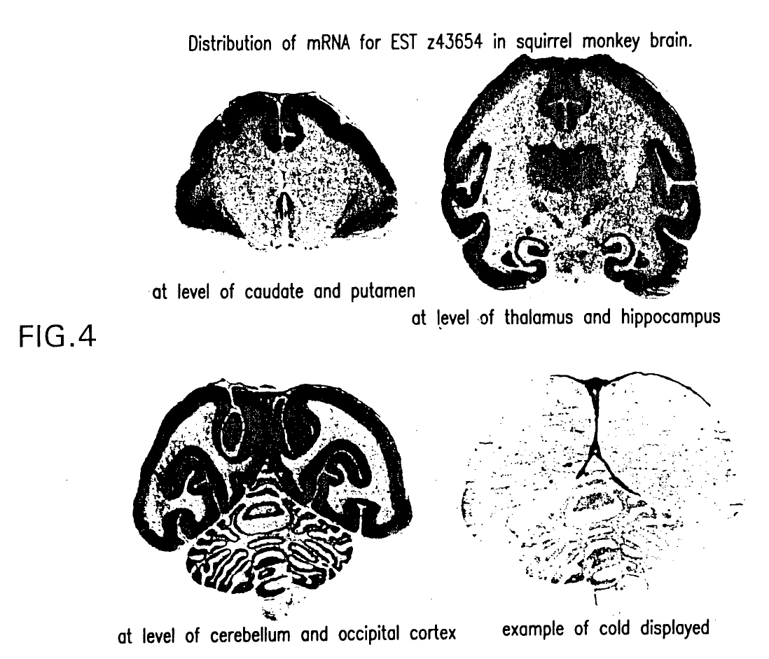

Highest levels of mRNA for HG20 were found in the hippocampus (dentate gyrus, CA3, CA2, and CA1). High levels were also seen in cortical regions (frontal, cingulate, temporal parietal, entorhinal, and visual) and the cerebellum, although medial septum, thalamic nuclei (medial-dorsal and lateral posterior), lateral geniculates, red nucleus, reticular formation, and griseum points all show expression of message. While there are many similarities with the distribution reported for the GABA_{B} receptor mRNA in rat, one marked difference is that expression of HG20 mRNA in the monkey caudate and putamen is below the level of detection while cortical levels are high. In the rat, the GABA_{B} receptor mRNA appears equally expressed in striatum as in cortex. FIG. 4 illustrates these results.

### EXAMPLE 5

Attempted Recombinant Expression of Full-Length HG20 Protein

Following the cloning of HG20 DNA, attempts were made to express full-length HG20 protein (941 amino acids) using various eukaryotic cell lines and expression vectors. The cell lines that were used were: COS-7 cells, HEK293 cells, and frog melanophores. The expression vectors that were used to attempt to express the full-length HG20 protein were: pCR3.1 and pcDNA3.1 (Invitrogen, San Diego, Calif.) and pciNEO (promega).

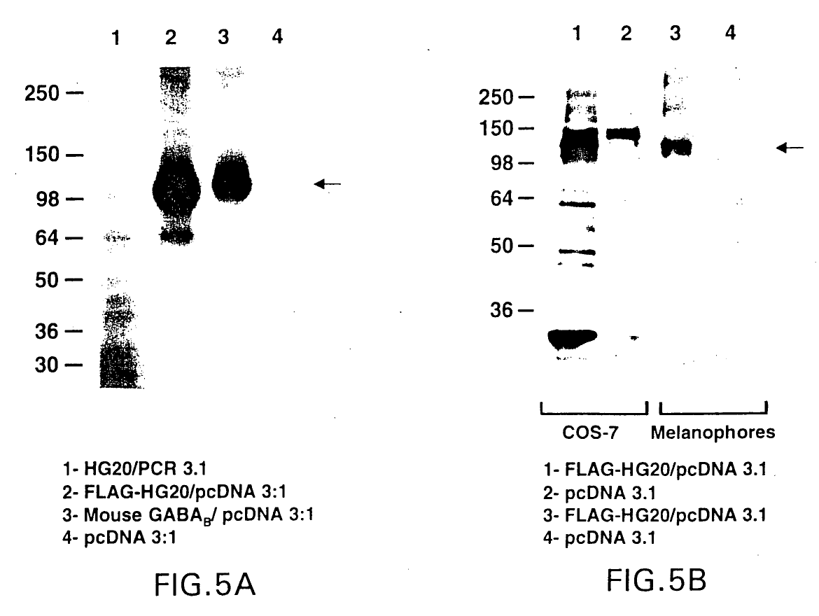

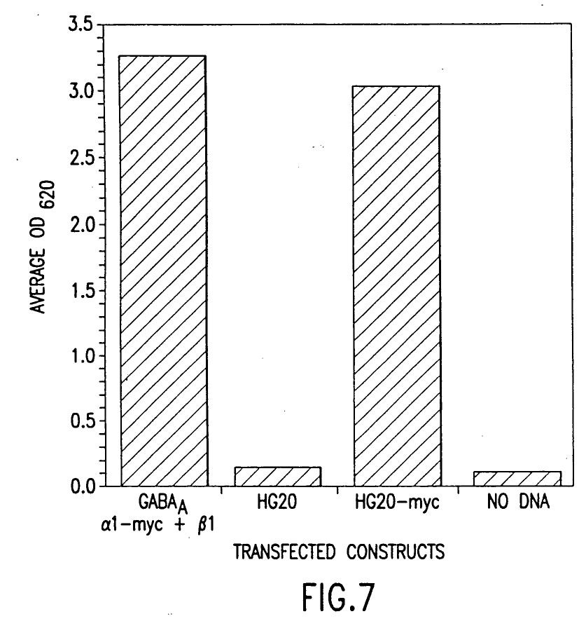

All of the attempts to express full-length HG20 described above were unsuccessful. See, e.g., FIG. 7, second bar from the left, marked “HG20.” See also FIG. 5A, lane 1. Although the reason for these failures is not known, it is possible that the highly GC rich nature of the region of the HG20 mRNA that encodes amino acids 1-51 results in the formation of secondary structure in the mRNA that impedes translation. It was only after the construction of an expression vector that encodes a truncated HG20 protein, lacking the first 51 amino acids, that HG20 was successfully expressed. FIG. 5A-B shows the results of the successful expression of an HG20 protein having amino acids 52-941. It is expected that expression of HG20 proteins having amino acids 53-941, 54-941, 55-941, etc., could be accomplished in a similar manner. It is also expected that expression of HG20 proteins having the above-described amino termini but having different carboxyl termini could be accomplished in a similar manner as well. Thus, the expression of an HG20 protein having an amino terminus as listed above and having a truncated carboxyl terminus could be accomplished. Alternatively, the carboxyl terminus could be fused to non-HG20 amino acid sequences, forming a chimeric HG20 protein. It is also possible to express HG20 having an amino terminus listed above as a chimeric protein with non-HG20 sequences fused to the amino terminus.

FIG. 5A shows the expression of amino acids 52-941 of HG20 as part of a chimeric or fusion protein with the FLAG epitope fused to the amino terminus of the HG20 sequences in a coupled in vitro transcription/translation experiment. FIG. 5B shows the expression of amino acids 52-941 of HG20 as part of a chimeric or fusion protein with the FLAG epitope fused to the amino terminus of the HG20 sequences in COS-7 cells and melanophores. The expression vector used in this experiment was pcDNA3.1. The expression constructs used in FIG. 5A-B also encoded a cleavable signal sequence from the influenza hemaglutinin gene that has been shown to facilitate the membrane insertion of G-protein coupled receptors (Guan et al., 1992, J. Biol. Chem. 267:21995-21998) and the fusion proteins were detected with anti-FLAG antibody. The expression constructs had also been engineered to contain a Kozak consensus sequence prior to the initiating ATG. The amino acid sequences of the hemaglutinin signal sequence and the FLAG epitope were:

| | |
|---|---|
| [MKTIIALSYIFCLVFA][DYKDDDDK] | SEQ.ID.NO:17 |
| HA signal peptide FLAG epitope | |

Amino acids 57-941 have been expressed in mammalian cells as part of a chimeric protein. A chimeric construct of HG20 was made that consisted of bases −224 to 99 of the bovine GABA_{A} α1 gene, a sequence encoding the c-myc epitope tag (amino acid residues 410-419 of the human oncogene product c-myc), a cloning site encoding the amino acid asparagine, and DNA encoding residues 57-941 of HG20. The resultant chimeric protein has the amino acid sequence shown below, with the construct cloned into pcDNA1.1Amp (Invitrogen, San Diego, Calif.).

(SEQ.ID.NO.:18)

Bovine alpha 1 signal seq c-myc
MKKSPGLSDYLWAWTLFLSTLTGRSYGQPSLQD EQKLISEEDL N
_res. 57-941 HG20_{—}
SIMGLMPLT . . .

The three periods “ . . . ” indicate that the chimeric protein sequence extends until amino acid 941 of HG20.

The cell surface expression of this construct was verified using a cell surface ELISA technique. Briefly, HEK293 cells were seeded at ˜1×10⁵ cells per well in a 24 well tissue culture plate and allowed to adhere for 24 hours. Each well was transfected with a total of 1 μg of DNA. In addition to tagged and un-tagged HG20 constructs, c-myc tagged GABA_{A} α1 was transfected with GABA_{A} β1 as a positive control for cell surface expression. Two days after transfection, the cells were assayed for surface expression of the c-myc epitope using the 9E10 monoclonal antibody raised to the c-myc epitope, followed by HRP (horse radish peroxidase) conjugated anti-mouse antibody (Promega) and colormetric development using K-Blue (Bionostics). The results are shown in FIG. 7. FIG. 7 demonstrates that when HG20 is part of a chimeric protein, it can be expressed well in mammalian cells but that when attempts are made to express full-length HG20 (amino acids 1-941) directly, i.e., not as part of a chimeric protein, essentially no expression is observed.

### EXAMPLE 6

Construction of Full Length Murine GABA_{B}R1a Coding Region

Using a combination of TFASTX (Pearson et al., 1997, Genomics 46:24-36) and TBLASTX (Altschul et al., 1997, Nucleic Acids Res. 25:3389-3402) searching programs against dbEST: Database of Expressed Sequence Tags (URL http://www.ncbi.nlm.nih.gov/dbEST/index.html), we identified partial cDNA clones in the EST collection which encoded murine GABA_{B}R1a using the rat GABA_{B} receptor subunit cDNAs (GenBank Accession Numbers Y10369 and Y10370) as probe sequences (Kaupmann et al., 1997, Nature 386:239-246). Two of these ESTs (IMAGE Consortium clone identification numbers 472408 and 319196) were obtained (Research Genetics, Birmingham, Ala.). The DNA sequences of both cDNA clones were determined using standard methods on an ABI 373a automated sequencer (Perkin-Elmer-Applied Biosystems, Foster City, Calif.).

The partial cDNAs were assembled by long accurate PCR using the following oligonucleotides: 472408 sense: 5′-GC GAATTC GGTACC ATG CTG CTG CTG CTG CTG GTG CCT-3′ (SEQ.ID.NO.:24), 472408 antisense: 5′-GG GAATTC TGG ATA TAA CGA GCG TGG GAG TTG TAG ATG TTA AA-3′ (SEQ.ID.NO.:25), 319196 sense: 5′-CCA GAATTC CCA GCC CAA CCT GAA CAA TC-3′ (SEQ.ID.NO.:26), 319196 antisense: 5′-CG GCGGCCGC TCA CTT GTA AAG CAA ATG TA-3′ (SEQ.ID.NO.:27) which amplified two fragments corresponding to the 5′ 2,100 basepairs and 3′ 1,000 basepairs of the murine GABA_{B}R1a coding region. The PCR conditions were 200 ng of cDNA template, 2.5 units of Takara LA Taq (PanVera, Madison, Wis.), 25 mM TAPS (pH 9.3), 50 mM KCl, 2.5 nM MgCl₂, 1 mM 2-mercaptoethanol, 100 mM each dNTP and 1 mM each primer with cycling as follows 94° C. 1 min, 9 cycles of 98° C. for 20 seconds, 72° C.-56° C. (decreases 2° C. per cycle), 72° C. for 30 seconds, followed by 30 cycles of 98° C. for 20 seconds, 60° C. for 3 minutes. A final extension at 72° C. for 10 minutes was performed. PCR products were cloned into the TA-Cloning vector pCRII-TOPO (Invitrogen, San Diego, Calif.) following the manufacturers directions. Cloned PCR products were confirmed by DNA sequencing. To form full-length cDNA, the pCINeo mammalian expression vector was digested with EcoRI and NotI. The EcoRI fragment from PCR cloning of 472408 and the EcoRI/NotI product from PCR cloning of 319196 were ligated in a three part ligation with digested pCINeo vector. The resulting clones were screened by restriction digestion with SstI which cuts once in the vector and once in the 472408 derived fragment. The resulting expression clone is 2,903 basepairs in length. The overall cDNA length, including untranslated sequences, inferred from the full length of the two ESTs is 4,460 basepairs.

### EXAMPLE 7

Preparation of Membrane Fractions

P2 membrane fractions were prepared at 4° C. as follows. Tissues or cells were washed twice with cold PBS, collected by centrifugation at 100×g for 7 min, and resuspended in 10 ml of buffer A: 5 mM Tris-HCl, 2 mM EDTA containing (1X) protease inhibitor cocktail Complete® tablets (Boehringer Mannheim), pH 7.4 at 4° C. Tissues or cells were disrupted by polytron homogenization, centrifuged at 100×g for 7 min to pellet unbroken cells and nuclei, and the supernatant collected. The resulting pellet was homogenized a second time in 10 ml of buffer A, centrifuged as described above and supernatant fractions saved. The pooled S1 supernatant was centrifuged at high speed (27 000×g for 20 min) and the pellet was washed once with buffer A, centrifuged (27 000×g for 20 min) and resuspended in buffer A to make the P2 membrane fraction, and stored at −80° C. Protein content was determined using the Bio-Rad Protein Assay Kit according to manufacturer instructions.

### EXAMPLE 8

Receptor Filter-Binding Assays

Competition of [¹²⁵I]CGP71872 binding experiments were performed with ˜7 μg P2 membrane protein and increasing concentrations of cold ligand (10⁻¹²-10⁻³ M). The concentration of radioligand used in the competition assays was 1 nM (final). Each concentration was examined in duplicate and incubated for 2 hours at 22° C. in the dark in a total volume of 250 μL binding buffer: 50 mM Tris-HCl, 2.5 mM CaCl₂ (pH 7.4) with (1X) protease inhibitor cocktail Complete®) tablets. Bound ligand was isolated by rapid filtration through a Brandel 96 well cell harvester using Whatman GF/B filters. Data were analyzed by nonlinear least-squares regression using the computer-fitting program GraphPad Prism version 2.01 (San Diego).

### EXAMPLE 9

Photoaffinity Labelling

P2 membranes were resuspended in binding buffer and incubated in the dark with 1 nM final concentration [¹²⁵I]CGP71872 (2200 Ci/mmol) in a final volume of 1 ml for 2 h at 22° C. The membranes were centrifuged at 27,000×g for 10 min and the pellet was washed in ice-cold binding buffer, centrifuged at 27,000×g for 20 min, resuspended in 1 ml of ice-cold binding buffer, and exposed on ice 2 inches from 360 nm ultraviolet light for 10 min. Photolabelled membranes were washed, pelleted by centrifugation, and solubilized in sample buffer (50 mM Tris-HCl pH 6.5, 10% SDS, 10% glycerol, and 0.003% bromophenol blue with 10% 2-mercaptoethanol). Samples were electrophoresed on precast NOVEX 10% Tris-glycine gels, fixed, dried, and exposed to Kodak XAR film with an intensifying screen at −70° C.

### EXAMPLE 10

Immunoprecipitation and Immunoblotting of GABA_{B} Receptors

Digitonin solubilized FLAG-tagged HG20 receptors were immunoprecipitated with a mouse anti-FLAG M2 antibody affinity resin (Kodak IBI) and immunoblot analysis conducted as previously described (Ng et al., 1996, Biochem. Biophys. Res. Comm. 227:200-204). Following washing of the immunoprecipitate, the pellet was resuspended in SDS sample buffer and subjected to SDS-PAGE and immunoblotted with affinity purified GABA_{B}R1a-specific antibodies 1713.1 (raised against the peptide acetyl-DVNSRRDILPDYELKLC-amide (a portion of SEQ.ID.NO.:20)) and 1713.2 (raised against the peptide acetyl-CATLHNPTRVKLFEK-amide (a portion of SEQ.ID.NO.:20)).

### EXAMPLE 11

Melanophore Functional Assay

Growth of Xenopus laevis melanophores and fibroblasts was performed as described previously (Potenza et al., 1992, Anal. Biochem. 206:315-322). The cells (obtained from Dr. M. R. Lerner, Yale University) were collected by centrifugation at 200×g for 5 min at 4° C., and resuspended at 5×10⁶ cells per ml in ice cold 70% PBS, pH 7.0. DNA encoding the relevant GPCR was transiently transfected into melanophores by electroporation using a BTX ECM600 electroporator (Genetronics, Inc., San Diego, Calif.). To monitor the efficiency of transfection, two internal control GPCRs were used independently (pcDNA1amp-cannabinoid 2 and pcDNA3-thromboxane A2; (Lerner, 1994, Trends Neurosci. 17:142-146)). Cells were electroporated using the following settings: capacitance of 325 microfarad, voltage of 450 volts, and resistance of 720 ohms. Following electroporation, cells were mixed with fibroblast-conditioned growth medium and plated onto flat bottom 96 well microtiter plates (NUNC). 24 hrs after the transfection, the media was replaced with fresh fibroblast-conditioned growth media and incubated for an additional day at 27° C. prior to assaying for receptor expression. For Gs/Gq-coupling responses (resulting in pigment dispersion), cells were incubated in 100 μl of 70% L-15 media containing 15 mM HEPES, pH 7.3, and melatonin (0.8 nM final concentration) for 1 hr in the dark at room temperature, and then incubated in the presence of melatonin (0.8 nM final concentration) for 1 h in the dark at room temperature to induce pigment aggregation. For Gi-coupled responses (resulting in pigment aggregation), cells were incubated in the presence of 100 μl/well of 70% L-15 media containing 2.5% fibroblast-conditioned growth medium, 2 mM glutamine, 100 μg/ml streptomycin, 100 units/ml penicillin and 15 mM HEPES, pH 7.3, for 30 min in the dark at room temperature to induce pigment dispersion. Absorbance readings at 600 nm were measured using a Bio-Tek Elx800 Microplate reader (ESBE Scientific) before (Ai) and after (Af) incubation with ligand (GABA; 1.5 hr in the dark at room temperature).

### EXAMPLE 12

Stable and Transient Transfections and Determination of cAMP Response in HEK293 Cells

HG20 and murine GABA_{B}R1a cDNAs were subcloned into pcDNA3.1 (Invitrogen, San Diego, Calif.) and used to transfect HEK293 cells. Stably expressing cells were identified after selection in geneticin (0.375 mg/ml) by dot blot analysis. For co-expression experiments, the stable cell lines hgb2-42 (expressing HG20) and rgb1a-50 (expressing murine GABA_{B}R1a) were transiently transfected with murine GABA_{B}R1a and HG20, respectively, in pcDNA3.1 and cells were assayed for cAMP responses.

Wild-type HEK293 cells, or HEK293 cells stably and transiently expressing HG20 and murine GABA_{B}R1a receptors were lifted in 1×PBS, 2.5 mM EDTA, counted, pelleted and resuspended at 1.5×10⁵ cells per 100 μl in Krebs-Ringer-Hepes medium (Blakely et al., 1991, Anal. Biochem. 194:302-308), 100 mM Ro 20-1724 (RBI) and incubated at 37° C. for 20 min. 100 μl of cells was added to 100 μl of prewarmed (37° C., 10 min) Krebs-Ringer-Hepes medium, 100 mM Ro 20-1724 without or with agonist and/or 10 μM forskolin. Incubations with GABA included 100 μM aminooxyacetic acid (a GABA transaminase inhibitor) to prevent breakdown of GABA and 100 μM nipecotic acid to block GABA uptake. Following a 20 min incubation at 37° C., the assay was terminated by setting the cells on ice and centrifuging at 2,000 rpm for 5 min at 4° C. 175 ml of assay solution was removed and replaced with 175 ml of 0.1 N hydrochloric acid, 0.1 mM calcium chloride and cells were set on ice for 30 min and then stored at −20° C. cAMP determinations were made using a solid phase modification (Maidment et al., 1989, Neurosci. 33:549-557) of the cAMP radioimmunoassay described by Brooker et al. 1979, Adv. Cyclic Nucl. Res. 10: 1-33) and previously reported in Clark et al., 1998, Mol. Endocrinol. 12:193-206). Immulon II removawells (Dynatech; Chantilly, Va.) were coated overnight with 100 μl of protein G (1 mg/ml in 0.1M NaHCO₃, pH 9.0) at 4° C. Prior to use, protein G-coated plates were rinsed with PBS-gelatin-Tween (phosphate buffered saline containing 0.1% gelatin, 0.2% Tween-20) 3 times quickly, and then once for 30 minutes. Following the rinse with PBS-gelatin-Tween, the RIA was set up by adding 100 μl 50 mM sodium acetate, pH 4.75, cAMP standards or aliquots from treated cells, 5,000-7,000 cpm ¹²⁵I-succinyl cAMP, and 25 μl of a sheep antibody to cAMP diluted in 50 mM sodium acetate, pH 4.75 (Atto instruments; dilution of stock to 2.5×10−⁵, determined empirically) to the plates in a final volume of 175 μl. Plates were incubated 2 hr at 37° C. or overnight at 4° C., rinsed 3 times with sodium acetate buffer, blotted dry, and then individual wells were broken off and bound radioactivity was determined in a gamma counter.

### EXAMPLE 13

In Situ Hybridization for Co-Localization Experiments

Preparation of rat brain sections, prehybridization and hybridization of rat brain slices was performed as described previously (Bradley et al., 1992, J. Neurosci. 12:2288-2302; http://intramural.nimh.nih.gov/lcmr/snge/Protocol.html). Adjacent coronal rat brain sections were hybridized with labeled antisense and sense riboprobes directed against HG20 (GenBank accession number AF058795) or murine GABA_{B}R1a.

HG20 probes were generated by amplification of HG20 with JC216 (T3 promotor/primer and bases 1172-1191) paired with JC217 (T7 promotor/primer and bases 1609-1626) or with JC218 (T3 promotor/primer and bases 2386-2405) paired with JC219 (T7 promotor/primer and bases 2776-2793):

| | | |
|---|---|---|
| (JC216: | cgcgcaattaaccctcactaaaggACAACAGCAAACGTTCAGGC; | (SEQ.ID.NO.:28) |
| JC217: | gcgcgtaatacg actcactatagggCATGCCTATGATGGTGAG; | (SEQ.ID.NO.:29) |
| JC218: | cgcgcaattaaccctcactaaagg CTGAGGACAAACCCTGACGC; | (SEQ.ID.NO.:30) |
| JC219: | gcgcgtaatacgactcactatagggGATGTC TTCTATGGGGTC;. | (SEQ.ID.NO.:31)) |

Murine GABA_{B}R1a probes were generated by amplification of murine GABA_{B}R1a with JC160 (T3 promotor/primer and bases 631-648) paired with JC161 (T7 promotor/primer and bases 1024-1041):

| | | |
|---|---|---|
| (JC160: | cgcgcaattaaccctcactaaaggAAGCTTATCCACCACGAC; | (SEQ.ID.NO.:32) |
| JC161: | gcgcgtaa tacgactcactatagggAGCTGGATCCGAGAAGAA. | (SEQ.ID.NO.:33)) |

For colocalization experiments, murine GABA_{B}R1a probes were labeled with digoxigenin-UTP and detected using a peroxidase-conjugated antibody to digoxigenin and TSA amplification involving biotinyl tyramide and subsequent detection with streptavidin-conjugated fluorescein. HG20 probes were radiolabelled (http://intramural.nimh.nih.gov/lcmr/snge/Protocol.html). For individual hybridizations, murine GABA_{B}R1a and HG20 riboprobes were radiolabeled with ³⁵S-UTP and detected as described previously (Bradley et al., 1992, J. Neurosci. 12:2288-2302; http://intramural.nimh.nih.gov/lcmr/snge/Protocol.html). Brain slices were either hybridized with individual radiolabelled probes or, for colocalization studies, simultaneously with probes to both murine GABA_{B}R1a and HG20 receptors.

Detection of the radiolabeled HG20 probe was performed after detection of the digoxigenin-labeled rgb1 probe on the same brain slices.

### EXAMPLE 14

Construction of N-Terminal and C-Terminal Fragments of Murine GABA_{B}R1a

The N-terminal fragment of murine GABA_{B}R1a comprising amino acid positions 1-625, was generated by PCR. The coding sequence of the N-terminal fragment was amplified by using primer pairs: NFP-CJ7843F139 (5′-ACC ACT GCT AGC ACC GCC ATG CTG CTG CTG CTG CTT CTG C-3′; SEQ.IS.NO.:34) and NRP-CJ7844 (3′-GG GTG CGA GCA ATA TAG GTC TTA AGG GTC GGC CGC CGG CGT CAC CA-5′; SEQ.IS.NO.:35). Similarly, the C-terminal fragment, amino acid positions 588-942, was generated by PCR using primer pairs: CFP-CJ7845 (5′-ACC ACT GCT AGC ACC GCC ATG CAG AAA CTC TTT ATC TCC GTC TCA GTT CTC TCC AGC-3′; SEQ.IS.NO.:36) and CRP-CJ7846 (3′-CAG CTC ATG TAA ACG AAA TGT TCA CTC GCC GGC CGC CGG CGT CAC CA-5′; SEQ.IS.NO.:37). PCR reactions were carried out using the Advantage-HF PCR kit (Clontech, Paolo Alto, Calif.) with 0.2 ng of murine GABA_{B}R1a DNA as the template, and 10 μM of each primer according to manufacturer instructions. The PCR conditions were as follows: precycle denaturation at 94° C. for 1 min, and then 35 cycles at 94° C. (15 s), annealing and extension at 72° C. (3 min), followed by a final extension for 3 min at 72° C. The PCR products, N-gb 1a and C-gb 1a DNA, flanked by Nhe1 and Not1 sites, were digested and subcloned into the Nhe1/Not1 site of pcDNA3.1 (Invitrogen, San Diego, Calif.).

### EXAMPLE 15

Cell Culture and Preparation of Membrane Fractions for Binding Experiments Using N-Terminal and C-terminal GABA_{B}R1a Fragments

COS-7 cells (ATCC) were cultured in DMEM, 10% bovine serum, 25 mM HEPES, and antibiotics and transiently transfected with murine gb1a/pcDNA3.1 (encoding full-length GABA_{B}R1a), N-gb 1a/pcDNA3.1 (encoding the N-terminal fragment of GABA_{B}R1a; see Example 14) or C-gb 1a/pcDNA3.1 (encoding the C-terminal fragment of GABA_{B}R1a; see Example 14) using Lipofectamine reagent (Gibco BRL) following the conditions recommended by the manufacturer. At 48 h post-transfection, P2 membrane fractions were prepared at 4° C. as follows: Cells were washed twice with cold PBS, collected by centrifugation at 100×g for 7 min, and resuspended in 10 ml of buffer A: 5 mM Tris-HCl, 2 mM EDTA containing (1X) protease inhibitor cocktail Complete® tablets (Boehringer Mannheim), pH 7.4 at 4° C. Cells were disrupted by polytron homogenization, centrifuged at 100×g for 7 min to pellet unbroken cells and nuclei, and the supernatant collected. The resulting pellet was homogenized a second time in 10 ml of buffer A, centrifuged as described above and supernatant fractions saved. The pooled S1 supernatant was centrifuged at high speed (27,000×g for 20 min) and the pellet was washed once with buffer A, centrifuged (27,000×g for 20 min), resuspended in buffer A to make the P2 membrane fraction, and stored at −80° C. Protein content was determined using the Bio-Rad Protein Assay Kit according to manufacturer instructions.

### EXAMPLE 16

In Vitro Transcription/Translation of GABA_{B}R1a and N-Terminal and C-Terminal Fragments

In vitro transcription coupled translation reactions were performed in the presence of [³⁵S]-methionine in the TNT Coupled Reticulocyte Lysate system (Promega, Wis.) using the pcDNA3.1 plasmid containing the full-length GABA_{B}R1a N-gb1a, and C-gb1a DNAs. Translation products were analysed by electrophoresis on 8-16% Tris-Glycine gradient gels (Novex pre-cast gel system) under denaturing and reducing conditions. Gels were fixed, treated with enlightening fluid (NEN), dried and exposed to Kodak X-AR film at −70° C. for 4 to 24 h. Analysis of the results of these in vitro transcription coupled translation reactions confirmed that the constructs whose production is described in Example 14 directed the expression of the appropriate GABA_{B}R1a fragments (see FIG. 17A).

### EXAMPLE 17

Immunoblot analysis for experiments with N-terminal and C-terminal fragments of GABA_{B}R1a

The expression of full-length and N-terminal and C-terminal GABA_{B}R1a fragments in vivo was confirmed by immunoblot analysis. Membranes were solubilized in SDS sample buffer consisting of 50 mM Tris-HCl pH 6.5, 10% SDS, 10% glycerol, and 0.003% bromophenol blue with 10% 2-mercaptoethanol and separated on SDS-PAGE. The full-length receptor and N-terminal fragment were detected using affinity purified rabbit GABA_{B}R1a polyclonal antibody 1713.1 (acetyl-DVNSRRDILPDYELKLC-amide; a portion of SEQ.ID.NO.:20) and 1713.2 (acetyl-CATLHNPTRVKLFEK-amide; a portion of SEQ.ID.NO.:20) (Quality Control Biochemicals (Hopkinton, Mass.). The C-terminal fragment was detected using a GABA_{B}R1a antibody raised against the C-terminal tail of the receptor (acetyl-PSEPPDRLSCDGSRVHLLYK-amide; SEQ.ID.NO.:20) (Chemicon Int., Inc., Canada).

### EXAMPLE 18

Receptor Filter-Binding Assays for Experiments with N-Terminal and C-Terminal Fragments of GABA_{B}R1a

Competition of [¹²⁵I] CGP71872 binding experiments were performed with ˜7 μg P2 membrane protein and increasing concentrations of cold ligand (10⁻12-10⁻3 M). The concentration of radioligand used in the competition assays was 1 nM (final). Each concentration was examined in duplicate and incubated for 2 hr at 22° C. in the dark in a total volume of 250 μL binding buffer: 50 mM Tris-HCl, 2.5 mM CaCl₂ (pH 7.4) with (1X) protease inhibitor cocktail Complete® tablets. Bound ligand was isolated by rapid filtration through a Brandel 96 well cell harvester using Whatman GF/B filters. Data were analyzed by nonlinear least-squares regression using the computer-fitting program GraphPad Prism version 2.01 (San Diego).

### EXAMPLE 19

Photoaffinity Labeling for Experiments with N-Terminal and C-Terminal Fragments of GABA_{B}R1a

P2 membranes were resuspended in binding buffer, and incubated in the dark with 1 nM final concentration [¹²⁵I]CGP71872 (2200 Ci/mmol) in a final volume of 1 ml for 2 h at 22° C. The membranes were centrifuged at 27,000×g for 10 min and the pellet was washed in ice-cold binding buffer, centrifuged at 27,000×g for 20 min and resuspended in 1 ml of ice-cold binding buffer and exposed on ice 2 inches from 360 nm ultraviolet light for 10 min. photolabelled membranes were washed and membranes pelleted by centrifugation and solubilized in sample buffer (50 mM Tris-HCl pH 6.5, 10% SDS, 10% glycerol, and 0.003% bromophenol blue with 10% 2-mercaptoethanol). Samples were electrophoresed on precast NOVEX 10% Tris-glycine gels, fixed, dried, and exposed to Kodak XAR film with an intensifying screen at −70° C.

### EXAMPLE 20

Construction of the Flag Epitope-Tagged Hg20 and Detection of Expression In Vitro and in COS-1 Cells

The FLAG epitope-tagged HG20 receptor subunit was constructed by PCR using a sense primer encoding a modified influenza hemaglutinin signal sequence (MKTIIALSYIFCLVFA; a portion of SEQ.ID.NO.:17) (Jou et al., 1980, Cell 19:683-696) followed by an antigenic FLAG epitope (DYKDDDDK; a portion of SEQ.ID.NO.:17) and DNA encoding amino acids 52-63 of HG20 and an antisense primer encoding amino acids 930-941 of the HG20 in a high-fidelity PCR reaction with HG20/pCR 3.1 as a template. HG20/pCR 3.1 is a plasmid that contains full-length HG20 (SEQ.ID.NO.:2) cloned into pCR3.1. The nucleotide sequences of the sense and antisense primers are: sense: 5′-GCC GCT AGC GCC ACC ATG AAG ACG ATC ATC GCC CTG AGC TAC ATC TTC TGC CTG GTA TTC GCC GAC TAC AAG GAC GAT GAT GAC AAG AGC AGC CCG CCG CTC TCC ATC ATG GGC CTC ATG CCG CTC-3′, (SEQ.ID.NO.:38); antisense: 5′-GCC TCT AGA TTA CAG GCC CGA GAC CAT GAC TCG GAA GGA GGG TGG CAC-3′. (SEQ.ID.NO.:39). The PCR conditions were: precycle denaturation at 94° C. for 1 min, 94° C. for 30 sec, annealing and extension at 72° C. for 4 min for 25 cycles, followed by a 7 min extension at 72° C. The PCR product, SF-HG20 DNA, flanked by NheI and XbaI sites was subcloned into the NheI/XbaI site of pcDNA3.1 (Invitrogen, San Diego, Calif.) to give rise to the expression construct SF-HG20/pcDNA3.1. The sequence of this construct was verified on both strands.

The SF-HG20 receptor was expressed in an in vitro coupled transcription/translation reaction using the TNT Coupled Reticulocyte Lysate system (Promega, Wis.) in the presence of [³⁵S]methionine according to the manufacturer instructions. Radiolabeled proteins were analyzed by electrophoresis on 8-16% Tris-Glycine gradient gels (Novex pre-cast gel system) under denaturing and reducing conditions. Gels were fixed and treated with Enlightening fluid (NEN), dried and exposed to Kodak X-AR film at −70° C.

COS-1 cells (ATCC, CRL 1650) were cultured in DMEM, 10% bovine serum, 25 mM HEPES, pH 7.4, and 10 units/mL penicillin-10 μg/mL streptomycin. Transient transfection of COS-1 cells with SF-HG20/pcDNA 3.1 was carried out using Lipofectamine reagent (Gibco BRL) following the conditions recommended by the manufacturer. At 48 h post-transfection, crude membranes were prepared and receptors were solubilized with digitonin and immunoprecipitated with anti-FLAG M2 affinity gel resin (IBI) under previously described conditions (Ng et al., 1993). The immunoprecipitate was washed and solubilized in SDS sample buffer, sonicated, electrophoresed, and blotted on to nitrocellulose membrane as described (Ng et al., 1993). The FLAG-tagged HG20 receptor was detected using an anti-FLAG antibody (Santa Cruz Biotech., Inc.) by following a chemilumescence protocol of the manufacturer (NEN).

### EXAMPLE 21

Kir Channel Activity in Xenopus Oocytes

With the following modifications, Xenopus oocytes were isolated as described (Hébert et al., 1994, Proc. R. Soc. Lond. B 256:253-261) from live frogs supplied by Boreal, Inc. After a brief (10 min) hypertonic shock with 125 mM potassium phosphate pH 6.5, oocytes were allowed to recover in Barth's solution for 1-2 hr. cDNA constructs for human Kir 3.1, Kir 3.2 channel isoforms (generous gifts from Dr. Hubert Van Tol, University of Toronto), and Giα1 (a generous gift of Dr. Maureen Linder, Washington University) were linearized by restriction enzymes and purified using Geneclean (Bio 101). Murine GABA_{B}R1a or FLAG-HG20 clones were subcloned into pT7TS (a generous gift of Dr. Paul Krieg, University of Texas) before linearization and transcription. Capped cRNA was made using T7 RNA polymerase and the mMessage mMachine (Ambion). Individual oocytes were injected with 5-10 ng (in 25-50 nL) of Kir3.1 and Kir3.2 constructs with mRNAs for murine GABA_{B}R1a or FLAG-HG20 and in combination with Giα1 as well. Kir currents were also evaluated in ooctyes co-injected with Kir3.1, Kir3.2, murine GABA_{B}R1a and FLAG-HG20 mRNAs. Currents were recorded after 48 hr. Standard recording solution was KD-98, 98 mM KCl, 1 mM MgCl2, 5 mM K-HEPES, pH 7.5, unless otherwise stated. Microelectrodes were filled with 3 M KCl and had resistances of 1-3 MW and 0.1-0.5 MW for voltage and current electrodes, respectively. In addition, current electrodes were backfilled with 1% agarose (in 3M KCl) to prevent leakage as described (Hébert et al., 1994, Proc. R. Soc. Lond. B 256:253-261). Recordings were made at room temperature using a Geneclamp 500 amplifier (Axon Instruments). Oocytes were voltage clamped and perfused continuously with different recording solutions. Currents were evoked by 500 msec voltage commands from a holding potential of −10 mV, delivered in 20 mV increments from −140 to 60 mV to test for inward rectifying potassium currents. Data were recorded at a holding potential of −80 mV and drugs were added to the bath with a fast perfusion system. Data collection and analysis were performed using pCLAMP v6.0 (Axon Instruments) and Origin v4.0 (MicroCal) software. For subtraction of endogenous and leak currents, records were obtained in ND-96, 96 mM NaCl, 2 mM KCl, 1 mM MgCl₂, 5 mM Na-HEPES and these were subtracted from recordings in KD-98 before further analysis.

### EXAMPLE 22

Radiation Hybrid Mapping of HG20

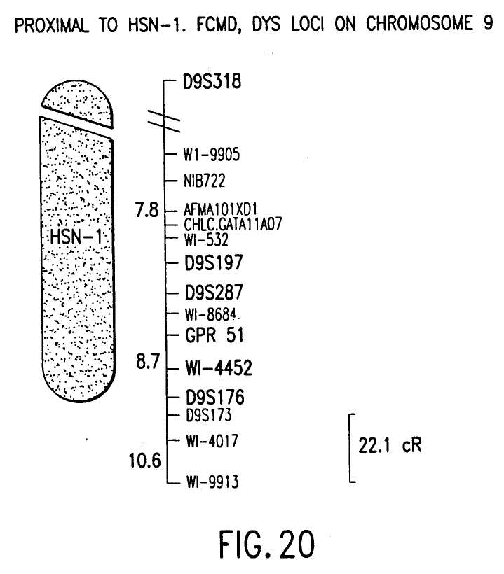

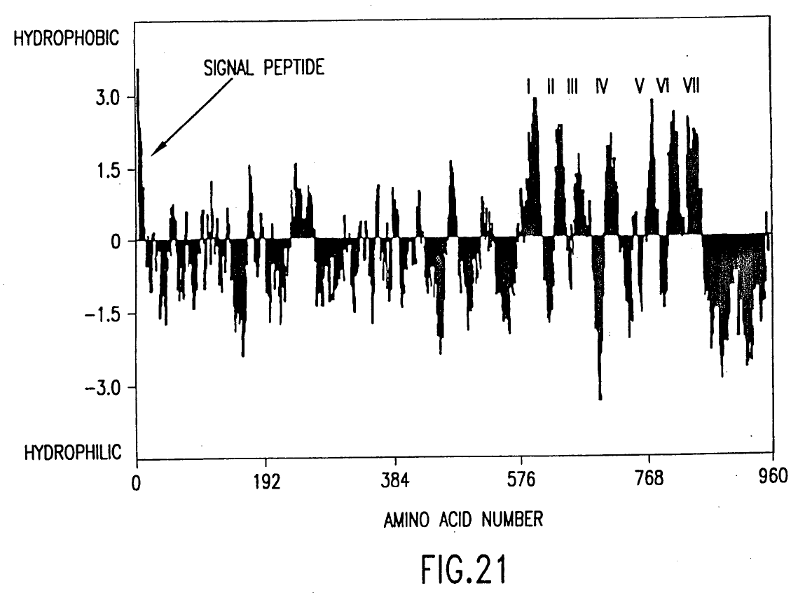

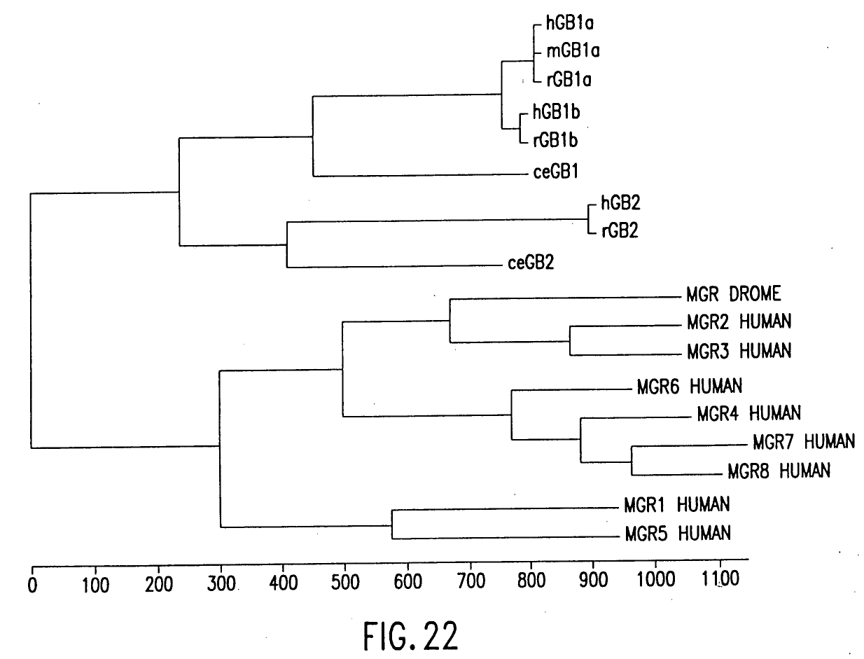

Radiation hybrid analysis assigned the HG20 gene to chromosome 9, placing it 4.81 cR from the WI-8684 marker on the GeneBridge 4 panel of 93 RH clones of the whole human genome. Searching of the OMIM database with D9S176 and D9S287 markers proximal to the HG20 gene revealed it to map proximal to the hereditary sensory neuropathy type 1 (HSN-1) locus, a ˜8 cM region flanked by D9S176 and 9S318 (Nicholson et al., 1996, Nature Genetics 13, 101-104) (FIG. 20). HSN-1 is the most common form of a group of degenerative disorders of sensory neurons characterized by a progressive degeneration of dorsal root ganglion and motor neurons that lead to distal sensory loss, distal muscle wasting and weakness, and neural deafness, among a number of other neuronally related deficits (Nicholson et al., 1996, Nature Genetics 13, 101-104). FCMD (Fukuyama congenital muscular dystrophy) and DYS (dysautonomia, another type of HSN) also map to this area. Candidate gene(s) in these disorders are likely critical to the development, survival, and differentiation of neurons.

A human BAC library was screened using the EcoRI fragment containing the full-length HG20 DNA, and end-sequencing was performed on BAC clones designated 6D18, 168K19, 486B24, and 764N4. The primer pair: ngf1t7+(5′-AAC AGT CAA AAC CCA CCC AG-3′; SEQ.ID.NO.:40) and ngf1t7-(5′-AAC AGT TTC CAG CTG TGC CT-3′; SEQ.ID.NO.:41) were identified for radiation hybrid mapping of the HG20 gene on the GENEBRIDGE 4 panel. BAC library screening and radiation hybrid mapping were performed by Research Genetics (Huntsville, Ala.).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of which are incorporated by reference in their entireties.

1. (canceled)

2. An isolated DNA molecule encoding an HG20 polypeiptide, said isolated DNA molecule comprising a nucleotide sequence selected from the group consisting of:
SEQ.ID.NO.:1;
Positions 293-3,115 of SEQ.ID.NO.:1;
Positions 317-3,115 of SEQ.ID.NO.:1;
Positions 395-3,115 of SEQ.ID.NO.:1;
Positions 398-3,115 of SEQ.ID.NO.:1;
Positions 404-3,115 of SEQ.ID.NO.:1;
Positions 407-3,115 of SEQ.ID.NO.:1;
Positions 416-3,115 of SEQ.ID.NO.:1;
Positions 422-3,115 of SEQ.ID.NO.:1;
Positions 428-3,115 of SEQ.ID.NO.:1;
Positions 446-3,115 of SEQ.ID.NO.:1; and
Positions 461-3,115 of SEQ.ID.NO.:1.

3. (canceled)

4. An expression vector comprising the DNA molecule of claim 2.

5. A recombinant host cell comprising the expression vector of claim 4.

6-15. (canceled)

16. A method of expressing an HG20 polypentide in cells, comprising:
(a) transfecting cells with.
an expression vector that expresses an HG20 polypeptide in the cells, wherein said expression vector comprises the isolated DNA molecule of claim 2; and
(b) culturing the transfected cells of step (a) cells under conditions such that the HG20 polypeptide is expressed.

17-19. (canceled)
